(19) United States
(12) Patent Application Publication
(10) Pub. No.: US 2018/0286272 A1
(43) Pub. Date: Oct. 4, 2018

(54) SYSTEM AND PROGRAM FOR COGNITIVE SKILL TRAINING

(71) Applicant: Atentiv LLC, Beverly, MA (US)

(72) Inventors: Ashley Frances MCDERMOTT, Cambridge, MA (US); Eric Bruce GORDON, Alton, NH (US); Jeremy George SOYBEL, Worcester, MA (US); Neal Alan GRIGSBY, Oakland, CA (US)

(21) Appl. No.: 15/755,881

(22) PCT Filed: Aug. 29, 2016
(86) PCT No.: PCT/US2016/049296

§ 371 Date: Feb. 27, 2018

Related U.S. Application Data
(60) Provisional application No. 62/211,269, filed on Aug. 28, 2015.

(51) Int. Cl. G09B 19/00; A61B 5/0482; A61B 5/16; A63F 13/85; A63F 13/67; A63F 13/69; A63F 13/46; G09B 5/02

(52) U.S. Cl. CPC G09B 19/00; A61B 5/0482; A61B 5/168; G09B 5/02; A63F 13/67; A63F 13/69; A63F 13/46; A63F 13/85

(57) ABSTRACT

This invention enables targeting, personalized measurement, and management of cognitive skills development by users, clinicians, teachers, and parents. The invention features a game based virtual learning curriculum for targeting and developing the underlying cognitive skills of executive functions. The methods and systems of the invention provide an effective and rapid video game-based training curriculum to improve the cognitive skills such as focused attention, sustained attention, cognitive inhibition, behavioral inhibition, selective attention, alternating attention, divided attention, interference control, novelty inhibition, delay of gratification, inner voice, motivational inhibition, and self-regulation. This curriculum utilizes: (i) each of the cognitive processes that underlie attention control and impulse inhibition; (ii) the identification of measurable and trainable cognitive skills; and (iii) game design and game mechanics that effectively train and enable retention of those skills. The game-based system provides a medical professional, clinician, parent, teacher and user with the ability to measure and manage training of targeted cognitive skills to reach a desired performance goal.

### BACKGROUND OF THE INVENTION

Executive functions are the cognitive processes that enable us to accomplish tasks such as organizing our thoughts, making future plans, and engaging in problem-solving. Executive function skills have been shown to be crucial in the foundation of learning and academic achievement.

In the course of infancy and normal early childhood development, the neural networks of executive functions naturally develop in young children, growing and refining into distributed neural networks. Difficulties with executive function development such as attention skills arise from dysfunctional cognitive processes in the neural networks, due largely to genetic inheritance followed by experiential events (Casey et al., Dev Psychobiol 40 (2002): 237-254). Underdevelopment of these cognitive skills prevents a child from being able to coherently function, like taking in and understanding new information or the completion of tasks in school. Foundational abilities, such as understanding and processing spoken language, require precise attention control. Both language processing and reading strongly rely upon attention skills to recognize key features in order to understand the message being expressed. Children with Specific Language Impairment, for instance, have been shown to have difficulty with selectively attending while listening to speech, causing them to miss important cues to word boundaries and meanings. While less closely studied, poor attention control has also been shown to be related to problems in arithmetic and solving word problems (Zentall et al., J Educ Psychol 82 (1990): 856-865).

Executive function difficulties play a prominent role in many learning disabilities and related problems. One of the core problems in many learning disabilities and, particularly, in Attention Deficit/Hyperactivity Disorder (ADHD), is difficulty with attention and impulse inhibition control. It has been found that the severity of ADHD symptoms is directly correlated with how much trouble children experience with academic achievement (Barry et al., J Sch Phychol 40 (2002): 259-283). The CDC reports that about 11% of children age 3-17 in the U.S. are diagnosed with ADHD, and according to the National Resource Center on ADHD, up to 50% of children with ADHD are diagnosed with at least one learning disability. They also estimate that between $36 and $52 billion is lost each year due to the loss in productivity for people who have ADHD.

To date, the most efficacious and best studied treatment for ADHD remains stimulant medication. While stimulant medications have been reliably shown to rapidly reach therapeutic benefit levels to improve behavior at home and in the classroom, these behavior improvements do not result in cognitive process improvements after taking medication and may last only 4 to 10 hours per dose. Any behavior benefits also appear to be lost after termination of medication use and come with many negative side effects, including headaches, nausea, suppressed appetite, insomnia, reduction in physical growth, and cardiovascular effects. Use of these stimulant medications have also led to the potential abuse of these drugs. In recent years, several non-stimulant medications have been approved for the treatment of ADHD. Studies indicate that his or her associated improvements are generally not as large as with stimulant medication, but do avoid some of the bigger risks associated with stimulant therapy, albeit introducing new side effects, such as acute suicidality and sedation.

Given the medication treatment landscape, there has been a great deal of interest in non-pharmaceutical treatments that achieve comparable effects and durability, with minimal side effects and risks of stigma and abuse. Traditional behavioral interventions, such as parent coaching and behavioral therapy, have been shown to have little effect on the course of ADHD, although they can help to manage some common co-morbidities, such as anxiety and depression. Another possibility is cognitive training, which involves doing tasks on a computer that are designed to train and strengthen specific cognitive abilities or skills, such as selective attention, inhibition control, or working memory. While this approach initially appeared to be a viable treatment, as research accumulates, studies with more strictly designed controls and meta-analyses doubt the effectiveness of these cognitive treatments for ADHD. The lack of efficacy was thought to be due to the current limited understanding of the relationship between ADHD and cognitive skills. Given the complexity of that relationship, it is advisable to not focus training on only one skill, but rather train, measure and manage a complete set of cognitive skills that children with ADHD struggle to use effectively.

Another treatment option is neurofeedback. Levels of cumulative EEG brainwaves in subjects with ADHD exhibit clear differences from the levels of EEG brainwaves recorded from people without ADHD (“normals”), including reduced levels of activity in the high-frequency brainwave bands (beta waves), and an increase in lower-frequency bands, especially theta waves from 4 to 7.5 Hz. Neurofeedback, also known as EEG biofeedback, provides a game-like feedback for a user to regulate his or her brainwaves, has been used with some success in reducing the behavioral symptoms severity of ADHD but not the underlying cognitive processes of executive functions. This training typically focuses on normalizing different aspects of the EEG signal based on broad populations, including the theta/beta ratio. Currently, the literature is divided about neurofeedback's effectiveness on reducing symptom severity. Significant limitations of neurofeedback is that the training is laborious and relies upon the subjects matching of his or her EEG signals to a ‘normal’ population template, which matching is dependent on wide complex variabilities. While a subject's attempt to regulate his or her brain activity may strengthen the parts of the brain that are most affected in ADHD, it is very difficult for a user to repeatedly manage his or her brain activity over time and most importantly neurofeedback does not isolate and target the underlying crucial cognitive processes of executive functions, which lead to learning and academic achievement.

There is a need for more effective learning systems to train, measure and manage the underlying cognitive skills of ADHD. Such systems could benefit users suffering from a wide variety of learning disabilities due to neuro developmental delays, such as patients with ADHD.

### SUMMARY OF THE INVENTION

This invention enables precise targeting, personalized measurement, and management of cognitive skills development by users, clinicians, teachers, parents, and other third parties. The invention features a learning curriculum embedded within a gaming software application that is utilized in conjunction with an EEG-based brain-to-computer interface (BCI) that measures a user's attention state level in real-time and enables the user to play/manage a video game by using their attention states to rapidly train themselves in the right cognitive skills. The invention is designed, e.g., to create a seamless experience accessible entirely by a user that integrates an empowering epic story line to increase the user's engagement (i.e., creating an intention to engage) and facilitate rapid learning of targeted cognitive skills.

In a first aspect, the invention features a method for training a cognitive skill (e.g., focused and sustained attention) in a user, the method including: (a) providing a computer-based virtual learning curriculum configured to train a targeted cognitive skill in the user, wherein the virtual training environment includes at least a first game module and a second game module, wherein the first game module includes a skill training module for training a targeted cognitive skill(s) and the second game module includes a skill transfer module configured to permit the user to demonstrate retention of the targeted cognitive skill(s) in a virtual learning environment separate from a learning environment of the skill training module (e.g., outside the skill training module); (b) measuring the EEG brain activity signals of the user and on the basis of the EEG brain activity signals calculating the attention state level of the user; (c) performing a skills training exercise in the skill training module, the skill training module including a first story line for advancing a user avatar toward completion of a mission while eliciting high and/or sustained attention state levels in the user, wherein an increase or decrease in the attention state level of the user produces a corresponding increase or decrease in the speed of the user avatar towards the completion of the mission (e.g., correspondingly increasing or decreasing the chance of achieving the challenge tasks, e.g., correspondingly increasing or decreasing skill learning (e.g., cognitive skill learning)); (d) during step (c), presenting challenge tasks to the user, wherein the challenge tasks are configured to train the targeted cognitive skill in the user; (e) during step (d), on the basis of the user response to the challenge tasks, calculating a skills performance score for the user and increasing the difficulty of achieving the challenge tasks when the skills performance score rises above a predetermined upper threshold and decreasing the difficulty of achieving the challenge tasks when the skills performance score falls below a predetermined lower threshold while the user avatar advances towards the completion of the training mission (e.g., as rapidly as possible under control of the user); and (f) following completion of the training mission (e.g., in each training module), performing a cognitive skill retention exercise in the skill transfer module, the skill transfer module including a second story line for presenting the retention challenge tasks to the user, wherein the retention challenge tasks are different from the challenge tasks presented in the skill training module, wherein the retention challenge tasks are configured for the user to demonstrate retention of the targeted cognitive skill (e.g., in a skill transfer module). In some embodiments, the first story line comprises a peer character, wherein the peer character provides guidance and motivation to the user to develop an inner voice in the user (e.g., dynamically provides guidance and motivation to the user avatar to achieve the desired goal by learning targeted cognitive skills while providing self-esteem or encouragement).

In some embodiments, the first story line and the second story line include a mentor character configured to encourage the user to engage in problem solving and to be self-motivated. In some embodiments, the mentor character is not configured to demonstrate the challenge task to the user. In certain embodiments, step (e) includes adjusting the difficulty of the challenge tasks based upon both the skills performance score and the attention state level of the user. Alternatively, step (e) can include adjusting the difficulty of the challenge tasks based upon the performance score or the attention state level of the user (e.g., solely on the skills performance score, independent of the attention state level). In some embodiments, step (e) further comprises adjusting the order of the targeted cognitive skills presented to the user avatar based upon the skills performance score and/or the attention state level of the user. In certain embodiments, the speed of a user avatar increases with increases in the attention state level or decreases with decreases in the attention state level. In still other embodiments, step (d) includes presenting challenge tasks to the user avatar at a rate that increases when the attention state level of the user increases. In some embodiments, step (d) comprises presenting challenge tasks to the user avatar at a rate that decreases when the attention state level of the user decreases. In other embodiments, step (d) includes presenting at least some challenge tasks (e.g., collection or collision avoidance challenge tasks and/or challenge tasks associated with attention or impulse/inhibition) to the user avatar only after the user has reached a predetermined threshold attention state level. In some embodiments, some challenge tasks are presented to the user avatar during a period of focused or sustained attention state levels. For example, step (d) can include presenting at least some challenge tasks to the user avatar only after the user has reached a predetermined threshold attention state level and only while the user maintains an attention state level above the predetermined threshold attention state level.

In some embodiments, step (f) further includes, on the basis of the user response to the challenge tasks presented in the skill transfer module, calculating a skill transfer score for the user, wherein achieving a skill transfer score above a predetermined threshold demonstrates transferability of the retained targeted cognitive skill and permits the user to advance to the next level of the computer-based virtual learning curriculum (e.g., including the training environment).

In some embodiments of any of the methods described herein, the skill training module is configured to train attention maintenance (e.g., focused attention and sustained attention) and the skill transfer module is configured for the user to demonstrate retention of the skill of attention maintenance (e.g., focused attention and sustained attention). In some embodiments, the method includes, following completion of the mission, calculating a focused attention score, a sustained attention score, or a cognitive inhibition score by identifying a number of attention state levels that are greater than a predetermined threshold attention state level (e.g., 50%, 55%, 60%, 65%, 70%, 75%, 80%, or 90%). In some embodiments, the method includes: (a) following completion of a mission, determining a number of attention state levels above a predetermined threshold attention state level; and (b) calculating a focused attention score from the number of attention state levels above the predetermined threshold attention state level. In some embodiments, the method includes, following completion of the mission, calculating a sustained attention score. In some embodiments, the method includes: (a) following completion of a mission, determining a duration of time during which attention state levels vary by less than a predetermined threshold variance; and (b) calculating a sustained attention state score from the duration of time during which attention state levels vary by less than a predetermined threshold variance (e.g., between 1% and 50%, e.g., 5%, 10%, 15%, 20%, 25%, or 30%, of the preceding attention state level). The sustained attention score can be calculated for sequential attention state levels greater than a predetermined attention state level (e.g., 50%, 55%, 60%, 65%, 70%, 75%, 80%, or 90%).

In some embodiments, the method includes: (a) following completion of a mission, determining (i) a number of correctly selected challenge tasks; (ii) a number of correctly rejected challenge tasks; (iii) a total number of challenge tasks; and (b) calculating a divided attention score from a composite of (i)-(iii). In some embodiments, a divided attention state score calculated by dividing the sum of (i) and (ii) by (iii).

In some embodiments, the skill training module is configured to train cognitive inhibition and the skill transfer module is configured to demonstrate retention of the skill of cognitive inhibition by the user. In some embodiments, the method includes: (a) determining a number of attention state levels over a predetermined threshold attention state level for a period of time (e.g., 10-120 seconds, e.g., 60 seconds) following the beginning of step (c); and (b) calculating a cognitive inhibition score from the number of attention state levels determined in part (a). In some embodiments, the predetermined threshold attention state level is 50%, 55%, 60%, 65%, 70%, 75%, 80%, or 90%.

In other embodiments, the skill training module is configured to train behavioral inhibition and the skill transfer module is configured to demonstrate retention of the skill of behavioral inhibition by the user. In some embodiments, the method includes: (a) following completion of the mission, determining (i) a number of correctly rejected challenge tasks; and (ii) a number of incorrectly selected challenge tasks; and (b) calculating a behavioral inhibition score from a composite of (i) and (ii). In some embodiments, a behavioral inhibition score is calculated, e.g., by dividing a number of correctly rejected targets (e.g., challenge tasks) by a sum of correctly rejected and incorrectly selected challenge tasks.

In other embodiments, the skill training module is configured to train selective attention and the skill transfer module is configured to demonstrate retention of the skill of selective attention by the user. In some embodiments, the method includes: (a) following completion of the mission, determining (i) a number of correctly selected challenge tasks; (ii) a number of correctly rejected challenge tasks; and (iii) a total number of challenge tasks; and (b) calculating a selective attention score from a composite of (i)-(iii). In some embodiments, a selective attention score is calculated, e.g., by dividing a sum of correctly selected and correctly rejected challenge tasks by a total number of challenge tasks).

In other embodiments, the skill training module is configured to train alternating attention and the skill transfer module is configured to demonstrate retention of the skill of alternating attention by the user. In some embodiments, the method includes: (a) following completion of the mission, determining (i) a number of correctly selected challenge tasks; and (ii) a number of correctly rejected challenge tasks, wherein the challenge tasks are presented immediately after a target rule switch (e.g., a challenge task immediately subsequent to a target rule switch); and (b) calculating an alternating attention score from a composite of (i) and (ii). In some embodiments, an alternating attention score is calculated, e.g., by dividing a sum of correctly selected and correctly rejected challenge tasks immediately after a switch by a number of total switches.

In other embodiments, the skill training module is configured to train novelty inhibition and the skill transfer module is configured to demonstrate retention of the skill of novelty inhibition by the user. In some embodiments, the method includes: (a) following completion of the mission, determining (i) a number of correctly selected challenge tasks; (ii) a number of correctly rejected challenge tasks; and (iii) a total number of challenge tasks; and (b) calculating a novelty inhibition score from a composite of (i)-(iii). In some embodiments, a novelty inhibition score is calculated, e.g., by dividing a sum of correctly selected and correctly rejected challenge tasks by a total number of challenge tasks.

In other embodiments, the skill training module is configured to train delay of gratification and the skill transfer module is configured to demonstrate retention of the skill of delay of gratification by the user. In some embodiments, the method includes: (a) following completion of the mission, determining (i) a number of correctly selected challenge tasks; and (ii) a total number of challenge tasks; and (b) calculating a delay of gratification score from a composite of (i) and (ii). In some embodiments, a delay of gratification score is calculated, e.g., by dividing a number of correctly selected challenge tasks by a total number of challenge tasks (e.g., challenge tasks presented within a predetermined time (e.g., 0.1 to 10 seconds, e.g., within 1, 2, 3, 4, 5, or more seconds) before or after a collision avoidance challenge task).

In other embodiments, the skill training module is configured to train self-regulation and the skill transfer module is configured to demonstrate retention of the skill of self-regulation by the user. In some embodiments, the method includes: (a) following completion of the mission, determining (i) a number of correctly selected challenge tasks; and (ii) a total number of challenge tasks; wherein the challenge tasks occur within a predetermined time (e.g., 0.1 to 10 seconds, e.g., within 1, 2, 3, 4, 5, or more seconds) before or after a collection or collision avoidance challenge task; and (b) calculating a self-regulation score from a composite of (i) and (ii). In some embodiments, a self-regulation score is calculated, e.g., by dividing a number of correctly selected challenge tasks by total challenge tasks.

In other embodiments, the skill training module is configured to train motivational inhibition and the skill transfer module is configured to demonstrate retention of the skill of motivational inhibition in a user. In some embodiments, the method includes: (a) following completion of the mission, determining (i) a number of correctly selected challenge tasks occurring after an incorrectly selected or an incorrectly rejected challenge task; (ii) a number of correctly rejected challenge tasks occurring after an incorrectly selected or an incorrectly rejected challenge task; (iii) a total number of correctly selected challenge tasks; (iv) a total number of correctly rejected challenge tasks; (v) a number of incorrectly selected challenge tasks; and (vi) a number of incorrectly rejected challenge tasks; and (b) calculating a motivational inhibition score from a composite of (i)-(vi). In some embodiments, a motivational inhibition score is determined by dividing a sum of correctly selected and correctly rejected challenge tasks after an incorrectly selected or incorrectly rejected challenge task by a sum of all correctly selected, correctly rejected, incorrectly selected, and incorrectly rejected challenge tasks.

In other embodiments, the skill training module is configured to train inner voice and the skill transfer module is configured to demonstrate retention of the skill of inner voice in a user. In some embodiments, the method includes: (a) following completion of the mission, determining a number of attention state levels greater than a preceding attention state level, wherein the preceding attention state level is less than a predetermined threshold attention state level; and (b) calculating an inner voice score from the number of attention state levels in part (a). For example, the inner voice score can be calculated by determining a number of positive changes in attention state level when the prior attention state level was less than a predetermined threshold attention state level. For example, the predetermined threshold attention state level may be 10%, 20%, 30%, 40%, 45%, 50%, 55%, 60%, 65%, or 70%. The positive changes can be positive changes of at least 10%, at least 20%, at least 30%, at least 40%, or at least 50%. In some embodiments, an interference control score is determined by dividing a number of incorrectly selected challenge tasks by a total number of challenge tasks.

In other embodiments, the skill training module is configured to train interference control and the skill transfer module is configured for the user to demonstrate retention of the skill of interference control. In some embodiments, the method includes: (a) following completion of an end goal, determining (i) a number of incorrectly selected challenge tasks; and (ii) a total number of challenge tasks; and (b) calculating an interference control score from a composite of (i) and (ii). For example, an interference control score can be calculated by dividing a number of incorrect selections (e.g., the number of individual challenge tasks (e.g., targets or clusters of targets) incorrectly selected by a total number of challenge tasks).

In some embodiments, the skill transfer module is configured to enable the user to demonstrate retention of the targeted cognitive skills learned in the skills training module. In some cases, the demonstration of retention corresponds to an increased chance of achievement of the desired goals or an increase in cognitive skill learning.

In some embodiments, the method further includes analyzing and reporting the skills performance of the user (e.g., the targeted cognitive skills performance).

Each module can include one or more levels, and each level can include one or more missions. The one or more levels can be levels of targeted cognitive skill development. The levels can be configured to teach the user targeted cognitive skills, including focused attention, sustained attention, cognitive inhibition, behavioral inhibition, selective attention, alternating attention, divided attention, interference control, novelty inhibition, delay of gratification, inner voice, motivational inhibition, and self-regulation. In certain embodiments, the method includes measuring, analyzing, and reporting the skills performance of the user (e.g., through mission performance reports or summary progress reports, e.g., to enable optimization and reduction in the severity of symptoms of ADHD).

In particular embodiments, the user has low attention and/or inhibition control (e.g., an attention deficit) or an inattention disorder. In other embodiments, the user has a low inhibition control or an inhibition disorder. The invented method can be performed at regular intervals, wherein steps (a) to (f) are repeated for at least one targeted cognitive skill, or steps (a) to (f) are repeated for two or more targeted cognitive skills. For example, the invented method can be performed at regular intervals from 3 to 7 times per week or more, for 10-60 minutes or more per session, over a course of 3-8 weeks, or more. For example, the method can be performed at regular intervals, such as 1, 2, 3, 4, or more times per week for 10, 20, 30, 40, 50, or 60 minutes, over a course of 3 or more weeks (e.g., to train, measure, and manage targeted cognitive skills development of the user to a desired level of reduced severity of the negative symptoms of ADHD).

In some embodiments, the method of any of the preceding methods, the skill training module comprises (i) providing a score of the user's skill performance, and (ii) on the basis of the score, selecting a difficulty level for the skill training module. In some embodiments, the user's skills performance is quantified by said user's accuracy in correctly distinguishing their activity between various stimuli.

In some embodiments, the method of any of the preceding claims further includes, during step (d), on the basis of the responses, (i) identifying impulsive responses by determining when the user is incorrectly responding to an impulse/inhibition challenge task or responding to a non-stimulus (e.g., impulsively or randomly responding, e.g., responding when not prompted to respond by a stimulus), and (ii) alerting the user to impulsive responses. In some embodiments, the alerting includes presenting the user with an audio or visual cue when the impulsive responses are identified. In some embodiments, step (d) includes calculating a skills performance score for the user on the basis of the user response to the challenge tasks, and step (e) includes reducing the skills performance score when the impulsive responses are identified. In some embodiments, the user's skills performance score is quantified using a combination of (i) the user accuracy in correctly distinguishing between various stimuli, and/or (ii) the ability of the user to avoid impulsive responses (e.g., to manage the skill improvements while driving symptom severity reductions to a desired level of behavior normality).

In some embodiments, the method of any of the preceding claims further includes, during step (d), identifying when the user is frustrated (e.g., with anxiety and/or depression) and triggering voice-over dialog from a peer character or mentor character. In some embodiments, the peer character or the mentor character provides reassurance and/or simple strategies for regulating emotional responses to feelings of frustration (e.g., with anxiety and/or depression).

In a related aspect, the invention features a game based system for training a targeted cognitive skill(s) in a user, the system including a processor equipped with an algorithm for presenting a computer-based virtual learning curriculum according to any of the preceding methods of the invention. In some embodiments, the algorithm is for presenting a computer-based virtual learning environment while the user is in a state of focused or sustained attention (e.g., having an attention state level of above 50%, 55%, 60%, 65%, 70%, 75%, 80%, or 90%). The game based system can further include an EEG headset for collecting EEG brain activity signals from the user.

In some embodiments of any of the preceding methods, the invention further includes (d) deriving an attention score for each of the attention-associated skills on the basis of the attention state level and/or the user response to the challenge task, wherein the attention-associated skills include focused attention, sustained attention, selective attention, alternating attention, or divided attention and deriving an attention or impulse/inhibition score for each of the attention- or impulse/inhibition-associated skills on the basis of the attention state and/or the user response to the challenge task, wherein the attention- or impulse/inhibition-associated skills include focused attention, sustained attention, cognitive inhibition, behavioral inhibition, selective attention, alternating attention, divided attention, interference control, novelty inhibition, delay of gratification, inner voice, motivational inhibition, or self-regulation; (f) for each training session, (i) calculating a global attention score derived from each of the attention scores; and/or (ii) calculating a global composite score derived from each of the attention or impulse/inhibition scores; and (g) determining, over the period of training, (i) a change in each attention score and a change in the global attention score; or (ii) a change in each attention and impulse/inhibition score and a change in the global composite score.

In another aspect, the invention features a method for training targeted cognitive skills in a user, the method including: (a) over a period of training including multiple training sessions, providing a computer-based virtual learning curriculum configured to train a plurality of attention-associated skills; (b) measuring the EEG brain activity signals of the user and on the basis of the EEG brain activity signals, calculating an attention state level of the user; (c) presenting a challenge task to the user, wherein the challenge task is configured to train one or more of the plurality of the attention-associated skills in the user; (d) deriving an attention score for each of the attention-associated skills on the basis of the attention state level and/or the user response to the challenge task, wherein the attention-associated skills include focused attention, sustained attention, selective attention, alternating attention, or divided attention; (e) for each training session, calculating a global attention score derived from each of the attention scores; and (f) determining, over the period of training, a change in each attention score and a change in the global attention score.

In another aspect, the invention features a method for training cognitive skills in a subject, the method including: (a) over a period of training comprising multiple training sessions, providing a computer-based virtual learning curriculum configured to train a plurality of targeted cognitive skills (e.g., focused attention, sustained attention, selective attention, alternating attention, or divided attention cognitive inhibition, behavioral inhibition, interference control, novelty inhibition, or motivational inhibition delay of gratification, inner voice, or self-regulation); (b) measuring the EEG brain activity signals of the user and on the basis of the EEG brain activity signals, calculating an attention state level of the user; (c) presenting a challenge task to the user, wherein the challenge task is configured to train one or more of the plurality of the targeted cognitive skills in the user; (d) deriving an targeted cognitive skill score for each of the targeted cognitive skills on the basis of the attention state level and/or the user response to the challenge task, wherein the targeted cognitive skills comprise focused attention, sustained attention, cognitive inhibition, behavioral inhibition, selective attention, alternating attention, divided attention, interference control, novelty inhibition, delay of gratification, inner voice, motivational inhibition, or self-regulation; (f) for each training session, calculating a global composite score derived from a composite of each of the targeted cognitive skill scores; and (g) determining, over the period of training, a change in each attention score, each impulse/inhibition score, and each self-regulation score; and/or a change in the global composite score.

In some embodiments of any of the preceding methods, the global attention score is a composite score (e.g., an average, or a weighted average) of the one or more cognitive skill scores. In some embodiments, the global composite score is a composite (e.g., an average, or a weighted average) of the one or more global attention scores and/or impulse/inhibition scores.

In some embodiments of any of the preceding methods, the global attention score is a composite (e.g., an average or a weighted average) of the attention scores. In some embodiments, the global composite score is a composite (e.g., an average or a weighted average) of the cognitive skill scores.

The invention features a method for treating an inattention and/or impulsivity disorder (e.g., Attention Deficit Hyperactivity Disorder, ADHD) in a user in need thereof, the method including: (a) providing a computer-based virtual learning curriculum configured to train a cognitive skill in the user, wherein the virtual training environment includes at least a first game module and a second game module, wherein the first game module includes a skill training module for training a cognitive skill and the second game module includes a skill transfer module configured to permit the user to demonstrate retention of the cognitive skill in a virtual learning environment outside the skill training module; (b) measuring the EEG brain activity signals of the user and on the basis of the EEG brain activity signals, calculating the attention state levels of the user; (c) performing a training exercise in the skill training module, the skill training module including a first story line for advancing a user avatar toward an end goal while eliciting high attention state levels in the user, wherein an increase or decrease in the attention state levels of the user produces a corresponding increase or decrease in the speed of the user avatar; (d) during step (c), presenting challenge tasks to the user to elicit responses from the user via an input device, wherein the challenge tasks are configured to train the targeted cognitive skill in the user; (e) during step (d), on the basis of the responses, (i) identifying impulsive responses by determining when the user is incorrectly selecting or responding (e.g., prematurely, randomly, or impulsively selecting or responding), and (ii) alerting the user to impulsive responses; and (f) following completion of the mission, performing a skill retention exercise in the skill transfer module, the skill transfer module including a second story line for presenting the challenge tasks to the user in a virtual learning environment different from the skill training module, wherein the challenge tasks are configured to demonstrate retention of the cognitive skill in the user. In some embodiments, the user is subjected to an immediate negative consequence when the impulsive responses are identified. The attention state level of the user can be scaled, e.g., from 0% to 100%, or from 0 to 100 points. Step (e) may further include (iii) adaptively providing similar challenge tasks for the user to recognize undesirable consequences of impulsivity and develop impulse inhibition (e.g., a desired impulse inhibition, e.g., by enabling the user to recognize undesirable consequences of impulsivity). The alerting can include presenting the user with an audio or visual cue when the impulsive responses are identified. In some embodiments, step (d) includes calculating a skills performance score for the user on the basis of the user response to the challenge tasks, and step (e) includes reducing the skills performance score when the impulsive responses are identified. In particular embodiments, step (e) includes on the basis of the user response to the challenge tasks, calculating a skill performance score for the user and increasing the difficulty of the challenge tasks when the skill performance score rises above a predetermined upper threshold and decreasing the difficulty of the challenge tasks when the skills performance score falls below a predetermined lower threshold while the user avatar advances towards the completion of a mission. For example, step (e) can include adjusting the difficulty of the challenge tasks based upon both the skills performance score and/or the attention state level of the user. For example, step (e) can include adjusting the difficulty of the challenge tasks based upon the performance score or the attention state level of the user (e.g., solely on the performance score, independent of the attention level). In some embodiments, step (d) includes presenting challenge tasks to the user at a rate that increases when the attention state level of the user increases or decreases when the attention state level of the user decreases. In some embodiments, step (d) includes presenting at least some challenge tasks to the user only after the user has reached a predetermined attention state level. For example, step (d) can include presenting at least some challenge tasks to the user only after the user has reached a predetermined threshold attention state level and/or only while the user maintains an attention state level above the predetermined threshold attention state level. Step (d) can include presenting challenge tasks to the user after the user has reached a predetermined threshold attention state level for a predetermined length of time. In some embodiments, the first story line includes a peer character presented to provide guidance and motivation to the user. The first story line and the second story line can include a mentor character presented to assist the user with problem solving and self-motivation, (e.g., without providing guidance as performed by the peer character). In some embodiments, step (f) further includes, on the basis of the user response to the challenge tasks presented in the skill transfer module, calculating a skills transfer score for the user, wherein achieving a transfer score above a predetermined threshold permits the user to advance to the next level of the computer-based virtual learning environment. In some embodiments, the skill training module is configured to train focused and sustained attention maintenance and the skill transfer module is configured to demonstrate retention in the user of the skill trained in the training module. In some embodiments, the skill training module is configured to train behavioral inhibition and the skill transfer module is configured for the user to demonstrate retention of the skill of behavioral inhibition. In some embodiments, the skill training module is configured to train selective attention and the skill transfer module is configured for the user to demonstrate retention of the skill of selective attention. In some embodiments, the skill training module is configured to train alternating attention and the skill transfer module is configured for the user to demonstrate retention of the skill of alternating attention. In some embodiments, the skill training module is configured to train novelty inhibition and the skill transfer module is configured for the user to demonstrate retention of the skill of novelty inhibition. In some embodiments, the skill training module is configured to train delay of gratification and the skill transfer module is configured for the user to demonstrate retention of the skill of delay of gratification. In some embodiments, the skill training module is configured to train self-regulation and the skill transfer module is configured for the user to demonstrate retention of the skill of self-regulation. In some embodiments, the modules are comprised of one or more levels, each level optionally being comprised of one or more missions (e.g., stages). The levels can be designed to teach the user cognitive skills, the cognitive skills including focused attention, sustained attention, cognitive inhibition, behavioral inhibition, selective attention, alternating attention, divided attention, interference control, novelty inhibition, delay of gratification, inner voice, motivational inhibition, or self-regulation. In particular embodiments, steps (a) to (f) are repeated for at least one cognitive skill, or steps (a) to (f) are repeated for two or more cognitive skills. In some embodiments, the user has ADHD and the method is performed by the user in an amount or frequency sufficient to reduce at least one of inattention, impulsivity, or hyperactivity in the user as measured by the ADHD rating scale. In particular embodiments, the method is performed in 3 to 7 sessions per week for 10 to 60 minutes per session, over a period of 3 or more weeks to treat at least one of inattention, impulsivity, or hyperactivity in the user. In some embodiments, the skill training module includes (i) providing a score of a user's performance, and (ii) on the basis of the score, selecting a difficulty level for the skill training module. In some embodiments, the user's skills performance score is quantified by (i) the user's accuracy in correctly distinguishing between various stimuli, (ii) the user to take correct actions, and/or (iii) the user to avoid incorrect actions (e.g., to avoid impulsive responses).

In a related aspect, the invention features a game-based system for treating an inattention and/or impulsivity disorder (e.g., ADHD) in a user in need thereof, the game-based system including a processor equipped with an algorithm(s) for presenting a computer-based virtual learning curriculum for performing any of the methods described herein. The game-based system can further include an EEG headset for collecting EEG data from the user to a computing and video display device.

In some embodiments, the invention features a game-based system for treating an inattention, impulsivity and hyperactivity disorder (ADHD) in a user in need thereof, the system including a reporting system illustrating the user training program adherence and cognitive skill levels retained at any point during the training program for performing any of the methods described herein. The reporting system can be, for example, a medical or clinical professional reporting system configured for use by a medical or clinical professional. Additionally or alternatively, the reporting system can be a non-clinical reporting system (e.g., a consumer or educational reporting system). For example, a reporting system can be adapted for use by a parent, guardian, teacher, or other non-medical professional, interested party, or consumer (e.g., to determine skills proficiency against a predetermined or relative level of performance). In some embodiments, the invention features a game-based system for treating an attention, impulsivity, and hyperactivity disorder in a user in need thereof, the system comprising a parent, teacher, user, or other interested party reporting system illustrating the user training program adherence and cognitive skill levels retained at any point during the training program, the system including a processor equipped with an algorithm for presenting a computer-based virtual learning curriculum according to any of the preceding methods.

In some embodiments, the invention features a game-based system for treating an inattention, impulsivity and hyperactivity disorder in a user in need thereof, the system including a reporting system illustrating the user's progress in developing the underlying cognitive skills of attention and impulsivity in a virtual learning curriculum, adherence to the learning curriculum, and targeted cognitive skill levels successfully demonstrated at any point during the learning curriculum.

The invention features a virtual learning curriculum for treating an attention, impulsivity and hyperactivity disorder (e.g., ADHD) in a user in need thereof, the system including a medical professional, teacher, user, parent, or interested party reporting system illustrating the user training program adherence and cognitive skill levels demonstrated and retained at any point during the training program for performing any of the methods described herein. The virtual learning curriculum can further include an EEG headset for collecting EEG data from the user and enabling the user to use their EEG data to communicate and guide outcomes in an adventure story or a series of epic stories. The virtual learning curriculum can further include a computer (e.g., a tablet, or smartphone, or any computing device) for recording and reporting the number of training sessions undertaken (or not undertaken) by a user and the length of the training session. In some embodiments, the virtual learning curriculum further includes a session planner for scheduling training sessions by a user and/or reminding the user of scheduled events.

In some embodiments of any of the preceding methods, the skill training module comprises (i) providing a score of a user's performance, and (ii) based on the score, selecting a difficulty level for the skill training module.

As used herein, the term “ability” refers to a user's cognitive ability(s) to take correct actions and inactions, to avoid incorrect actions and inactions for the purpose of achieving a challenge task.

As used herein, the term “brain-to-computer interface” or “BCI” refers to a communication pathway between a user's brain activity and a receiving device. An encephalography instrument assists in facilitating this brain activity interface between the user and the processor that is connected to the game elements of the virtual learning curriculum and provides and EEG-based measure of the user's attention (e.g., an attention level scaled from 0-100%, with 100% being the user's highest attention level and 0% being the user's lowest attention level).

As used herein, the term “skill training module” refers to a type of video game learning module designed to teach one or several targeted cognitive skills within a virtual fantasy world. For example, the user may enter this module first upon beginning the first mission of the video game and then each succeeding mission. The skill training module is configured to train one or more of the user's cognitive skills in an entertaining and rapid manner.

As used herein, the term “skill transfer module” refers to a type of video game learning module entered after completion of the skill training module in a level or mission of the video game. The skill transfer module is configured to reinforce and demonstrate to the user the targeted cognitive training skills taught in the preceding skill training module(s). The skill transfer module is a game module for presenting cognitive skill retention exercises to the user following the performance by the user of a training module for training the targeted cognitive skill. The skill transfer module presents the cognitive challenge tasks in a context and/or environment that is different from the training module to demonstrate and report the adaptability of the targeted skill in real life and maximize retention for later use. For example, the skill training module can be presented as a testing environment in which cognitive challenge tasks are reviewed for multiple different uses to manage skills optimization.

As used herein, the term “inattention disorder” refers to a condition characterized by inattention, over-activity, and/or impulsiveness. The methods and systems of the invention can be useful for treating attention disorders (i.e., improving one or more symptoms of the disorder following a training regimen described herein), such as, without limitation, Attention Deficit Hyperactivity Disorder, Attention Deficit Disorder, and Hyperkinetic Disorder. Attention Deficit Hyperactivity Disorder, which is also referred to in the literature as Attention Deficit Disorder/Hyperactivity Syndrome (ADD/HS), is a condition (or group of conditions) characterized by impulsiveness, distractibility, inappropriate behavior in social situations and hyperactivity (American Psychiatric Association. Diagnostic and Statistical Manual of Mental Disorders: DSM-5. ManMag, 2013). A particularly severe form of ADHD is termed Hyperkinetic Disorder.

As used herein, “alternating attention” is the mental flexibility of being able to rapidly shift attention from one object of attention to another object of attention (e.g., as part of a single challenge task or between multiple challenge tasks).

As used herein, “attention level” or an “attention state level” refers to the output value given by the EEG device according to one or more parameters derived from EEG brain activity signals.

As used herein, “attention maintenance” is the ability to focus on a stimulus for an extended period of time with sustained vigilance.

As used herein, “behavioral inhibition” refers to the ability to inhibit or suppress a pre-potent learned response when that response would be inappropriate in the given context.

As used herein, a “challenge task” refers to a game element within a virtual learning curriculum requiring a user response that is configured to teach one or more cognitive skills in a user. A challenge task can be a target or a cluster of targets to which the user is instructed to respond (e.g., by selecting or rejecting the target) according to a criteria (i.e., a target rule). Alternatively, challenge tasks can be collision avoidance tasks, such as a requirement to dodge an obstacle or jump a hurdle, for example. A third type of challenge task is a collection challenge task, which may require a user to collect an item (e.g., a token or a crystal). An “impulse/inhibition challenge task” refers to a challenge task configured for teaching impulse/inhibition control in a user. An impulse/inhibition challenge task can involve a delay between the introduction of a target and the user's ability to apply the target rules to the target (e.g., a shape or symbol may not be immediately presented). In this case, if the user responds to the target prior to presentation of its target rule, that response is incorrect and is classified as an impulsive response. A user's response to a challenge task or to any combination of challenge tasks can be used in the calculation of an attention score or an attention and impulse/inhibition score.

As used here, a “retention challenge task” refers to a game element requiring a user response that is configured to demonstrate retention of a targeted cognitive skill trained in the user.

As used herein, “delayed gratification” and “delay of gratification” are used interchangeably and refer to the ability to inhibit or suppress an action that would result in an immediate reward in order to gain a larger reward later.

As used herein, “dynamic” guidance or to “dynamically guide” refers to the characteristic that the occurrence and/or type of guidance depends on the skill performance or attention state level of the user. For example, guidance may occur more frequently if the user is struggling to maintain attention.

As used herein, “mentor character” refers to a character presented to the user during game play that provides wisdom, objectivity and vision but not the guidance to the user that the peer character provides the user avatar. A mentor character can be, for example, a voice-over character that tells the user what to do but not how to do the activity. Therefore, the mentor character can be configured to enable the user avatar to demonstrate the skills retained unaided by any character. The terms “mentor character” and “mentor avatar” are used interchangeably herein.

As used herein, “novelty inhibition” refers to the ability to recognize when a novel stimulus is irrelevant, and to subsequently ignore it and return to the current challenge task or goal.

As used herein, “peer character” refers to a character presented to the user during game play that provides encouragement and contextual help to the user to succeed at challenge tasks presented during game play (e.g., during a skill training module). The terms “peer character” and “peer avatar” are used interchangeably herein.

As used herein, “performance score” or “skills performance score” refers to a score calculated for and assigned to a user and developed for a user, medical professional, teacher, adult, or any third party to present cognitive skills proficiency based on response to challenge tasks presented in the skill training module, alone or in combination with attention state level measurements.

As used herein, “selective attention” refers to the ability to process or focus attention on the specific stimulus that is relevant to pertinent goals while ignoring irrelevant stimuli.

As used herein, “self-regulation” refers to the ability to remain goal-oriented, motivated, and organized while constantly monitoring and assessing one's own behavior.

As used herein, “skill retention exercise” refers to a task or a series of challenge tasks within the skills transfer module that require a user to use the same cognitive function that was recently required by a training exercise (e.g., during a skill training module).

As used herein, “skill training module” refers to a mission in the game that requires use of a cognitive skill to progress to its corresponding skill transfer module. A skill training module can be a type of virtual learning curriculum uniquely designed to teach one or several cognitive skills within a series of story adventure missions. The user can enter a skill transfer module as a user avatar first upon beginning the first mission of the virtual learning curriculum and advances to each succeeding mission. The skill training module can be configured to train the user's cognitive skills in an entertaining and rapid manner.

As used herein, “skill transfer module” refers to a mission in the game that tests the user's development of one or more of the cognitive skills trained by the preceding skill training module(s). A skill transfer module can be a type of virtual learning curriculum entered after completion of the skill training module in any mission of the video game. The skill transfer module is can be configured to reinforce and demonstrate to the user, medical professional, teacher, parent, or any third party the targeted cognitive training skills learned and taught in the preceding skill training module(s). The skill transfer module can be a game module for presenting cognitive skill retention exercises to the user following the performance by the user of a training module for training the targeted cognitive skill. The skill transfer module can present the cognitive challenge tasks in a context and/or environment that is different from the training module to demonstrate adaptability of the targeted skill in real life and maximize retention for later use.

As used herein, “training exercise” refers to a challenge task or a series of challenge tasks that require a user to exercise a cognitive skill during a skill training module.

As used herein, “transfer score” refers to a score calculated for and assigned to a user based on response to challenge tasks presented in the skill transfer module.

As used herein, the term “electrical sensor” refers to a sensor used for measuring bioelectric signals, such as EEG or EMG signals. The electrical sensor can include one or more electrodes, optionally formed from a flexible conductive fabric.

Other features and advantages of the invention will be apparent from the following Detailed Description, the drawings, and the claims.

### BRIEF DESCRIPTION OF THE DRAWINGS

FIG. 1 is a representative graph illustrating the roles of the peer, the mentor; attention state levels and the feedforward elements of the learning curriculum.

FIG. 2 is a representative graph of the duration of benefits as a function of completing the virtual learning curriculum.

FIG. 3 is a drawing depicting an example of the kind of environment and tasks in which “Attention Maintenance” skills are trained and assessed in the training module.

FIG. 4 is a drawing depicting an example of the type of environment and tasks in which “Behavioral Inhibition” skills are trained and assessed in the training module.

FIG. 5 is a drawing depicting an example of the type of environment and tasks in which “Selective Attention” skills are trained and assessed in the training module.

FIG. 6 is a drawing depicting a first example of the type environment and tasks in which “Alternating Attention” skills are trained and assessed in the training module.

FIG. 7 is a drawing depicting a second example of the type of environment and tasks in which “Alternating Attention” skills are trained and assessed in the training module.

FIG. 8 is a drawing depicting an example of the type of environment and tasks in which “Novelty Inhibition” skills are trained and assessed in the training module.

FIG. 9 is a drawing depicting an example of the type of environment and tasks in which “Inner Voice” skills are trained and assessed in the training module.

FIG. 10 is a drawing depicting a first example of the type of environment and tasks in which “Self-Regulation” and “Delay of Gratification” skills are trained and assessed in the training module.

FIG. 11 is a drawing depicting a second example of the type of environment and tasks in which “Self-Regulation” and “Delay of Gratification” skills are trained and assessed in the training module.

FIG. 12 is a drawing depicting an example of the type of different environment and tasks in which the user learns and demonstrates the transfer of targeted cognitive skills learned in the training module(s).

FIG. 13 is a diagram showing the flow of participants through a clinical study.

FIG. 14 is a graph showing the mean ADHD-Rating Scale (ADHD-RS) combined scores for trained and control groups before and after the initial 8 weeks of intervention, as reported by clinicians and parents. Error bars are standard error.

FIG. 15 is a graph showing the mean number of questions answered correctly on the Permanent Product Measure of Performance (PERMP) before and after the initial 8 week (Wk) intervention period. Error bars are standard error.

FIG. 16 is an exemplary Mission Performance Report (MPR) for a training session of a user.

FIG. 17 is a Mission Performance Report of Mission 2 for the case study presented in Example

FIG. 18 is a Mission Performance Report of Mission 4 for the case study presented in Example

FIG. 19 is a Mission Performance Report of Mission 8 for the case study presented in Example

FIG. 20 is a Mission Performance Report of Mission 12 for the case study presented in Example 16.

FIG. 21 is a Mission Performance Report of Mission 14 for the case study presented in Example 16.

FIG. 22 is a Mission Performance Report of Mission 15 for the case study presented in Example 16.

FIG. 23 is a Summary Progress Report for the case study presented in Example 16.

### DETAILED DESCRIPTION

The invention features a video game based virtual learning curriculum (e.g., pedagogy) for targeting and developing cognitive skills (e.g., cognitive skills underlying a person's executive functions). The methods and systems of the invention provide an effective and rapid video game-based learning curriculum to improve the multiple cognitive skills underlying executive functions, such as attention and impulse control. This curriculum utilizes: (i) the cognitive skills and processes that underlie attention control; (ii) the identification of measurable, trainable and manageable cognitive skills that utilize those processes; and (iii) game design and game mechanics that effectively train those skills for use later in life. The invention enables precise targeting, personalized measurement and management of cognitive skills development by users, clinicians, teachers and parents.

The presently described virtual game (i.e., learning curriculum) utilizes a feedforward modeling system to train, measure and manage targeted cognitive skill(s) in a user during the skills training and transfer modules. Feedforward modeling occurs when a desired goal is anticipated or visualized and the individual brings forward their resident cognitive skill levels to achieve the desired goal. Thus, the distinctive elements of a feedforward modeling system is engagement, goal development, anticipation of goal achievement and commission of the act or not. The learning curriculum embedded in a game's virtual world induces feedforward modeling of cognitive processes in a user's brain. Specifically, by coupling dynamic challenge tasks with heightened attention state levels, the game exercises neural circuitry corresponding to those desired attention states levels. The user anticipates the rewards offered by the game narrative and brings his or her attention to the state level required to attain those rewards. As the game progresses, it adapts to the user's changing attention state level so as not to become too easy or too difficult for the user. This ensures optimal feedforward dynamics for each user and maximal neural stimulation in attention-associated regions of the brain.

The invention has been inspired by the last two decades of research that has yielded great promise for the development of neuropsychological methods that would link the nervous system to cognitive dysfunction and therefore a pathway (i.e., a closed loop between the behavioral disorder and the underlying problems in neurological circuits to the development of sustained treatments for neurodevelopmental delay disorders due to deficits in cognitive skills learning (i.e., executive function disorders such as ADHD.) Although the results have been complex, and sometimes contradictory, it has illustrated links between the complexity and heterogeneity of ADHD and the neurocognitive dysfunctions. It has revealed that mental illnesses are circuit disorders requiring natural circuitry corrections not chemical balancing and that neuroplasticity-based treatments will be an important part of future ‘Best Practices’ in neurological and psychiatric medicine (Insel et al Scientific American 302.4 (2010): 44-51). We know that our brains physically change based on what we learn. Circuits in our brain that we use strengthen and those we don't use eventually disappear (i.e., neuroplasticity; Merzenich et al., Neuroplasticity and Neurorehabilitation (2015):6). There is a critical/sensitive period during childhood. During this critical period, children learn from exposure. If they are not exposed enough to a certain skill or their brain does not develop the correct circuits, they will need specific training and exposure (Blakemore et al., Journal of Child Psychology and Psychiatry 47.3-4 (2006): 296-312). After this critical period, changes or corrections to circuits require specific high levels of attention. In late childhood and beyond into adulthood, to learn something new and change, the brain requires specific heightened focus of attention on what one wants to learn (Merzenich et al., Front Human Neurosci 27 (2014): 385; Polley et al., J Neuroci 3 (2006): 4970-82). Paying attention sets the brain up to learn and change. Attention is a trigger to the brain to release acetylcholine from the nucleus basalis to make the brain “ready to learn” (Grossberg et al., Front Neurosci 20 (2016): 501; Polley et al., J Neuroci 3 (2006): 4970-82; Murray et al., Neuroscience 14 (1985): 1025-32; Robbins et al., Kilgard et al., Science 13 (1998): 1714-8). Rewarding experiences control what is learned. Rewards cause a release of dopamine from the basal ganglia that signals what should be learned by facilitating long-term potentiation (Merzenich et al., Front Human Neurosci 27 (2014): 385; Reynolds et al., Nature 6 (2001): 67-70; Reynolds et al., Neuroscience 99 (2000): 199-203).

The methods and systems of the invention can be incorporated into a dynamic closed loop neuropsychological methodology to rapidly teach, measure and manage the underlying cognitive skills of otherwise naturally developed executive functions, beginning with attention and inhibition control. The methods and systems of the invention can be used in the clinic, school, home, workplace, etc. Fundamentally, the methodology can place a user in an optimized learning zone or environment where the neurobiology of the brain's learning capacity is activated simultaneously with the novelty provided by highly engaging challenge tasks that effectively teach and promote retention of cognitive skills for transfer to home, school, work and life. The combination naturally activates s on the user's neuroplastic process in developing new and strengthened brain circuits to retain newly developed cognitive skills. The methods and systems of the invention can include a personalized calibration of a user's attention state levels for use and real time measurement within an epic adventure story, a virtual learning curriculum for cognitive skill development. By the user maximizing their attention levels to drive to adventure story mission completion, the user can rapidly enter this learning zone to build and strengthen circuits while experiencing dynamic modeling of skill challenge tasks that teach 13 underlying cognitive skills. Challenge tasks within the story line can be dynamically adjusted to precise measurements of personal skill performance levels and attention state levels and lead a user through a virtual learning curriculum for targeting and further developing the underlying cognitive skills of executive functions. The learning methodology in the literature is referred to as feed forward learning and the methods and systems of the invention uniquely combines this approach with the teaching of dynamically modeled cognitive skills. Therefore, it is called feed forward modeling. The game-based system can provide a medical professional, clinician, parent, teacher, or user with a closed loop system that can reverse the severe symptoms of ADHD (i.e., inattention and impulsivity) by precisely targeting, measuring and managing the training of cognitive skills to achieve normalized symptomology levels characteristic of non ADHD children and adults.

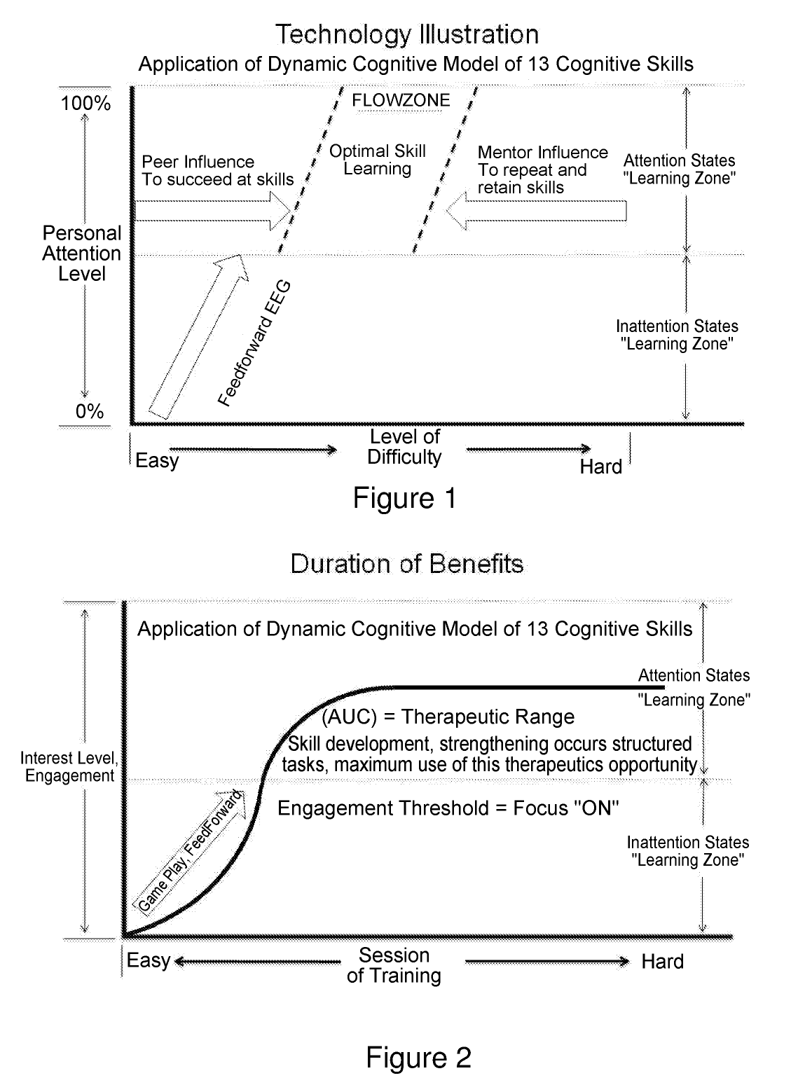

The role of feedforward modeling in cognitive skill development and the video game-based learning curriculum described herein is illustrated in FIGS. 1 and 2.

### Cognitive Skills

The cognitive processes that underlie attention include, without limitation, eight critical attention, inhibition, and self-regulatory skills, presented in Table 1 below.

TABLE 1
8 Cognitive skills underlying attention and impulse inhibition

| Skill | Definition |
|---|---|
| Attention Maintenance | The ability to focus on a stimulus for an extended time with sustained high |
| | attention level. |
| Selective Attention | The ability to decide where to focus one's attention and avoid distractions |
| | in a “noisy distracting” environment. |
| Alternating Attention | The ability to shift one's focus of attention rapidly or Mental flexibility. |
| Behavioral Inhibition | The ability to suppress a pre-potent or impulsive response. |
| Novelty Inhibition | The ability to inhibit a response to novelty and minimize distraction due to |
| | new stimuli |
| Delay of Gratification | The ability to suppress the desire for an immediate reward in order to gain |
| | a larger reward later. |
| Inner Voice | The ability to use an inner monologue to motivate oneself and guide |
| | oneself through problem solving. |
| Self-Regulation | The ability to regulate your actions in response to future goals and past |
| | outcomes: make a plan, execute on it, reflect on the plan, and revise if |
| | necessary. |

These composite eight cognitive skills can be further refined into a total of thirteen cognitive skills to target and train additional nuances in a user's attention and impulsivity inhibition skills. Specifically, attention maintenance can be separated into focused attention and sustained attention. Selective attention can be divided into selective attention and interference control, processes that can be activated simultaneously to selectively focus on one stimulus while suppressing distractions from other stimuli. Alternating attention can additionally include the skill of divided attention, as these processes both rely on the ability to rapidly shift focus between multiple stimuli and tasks. Building upon the skill of behavioral inhibition is motivational inhibition, referring to the ability of a child to effectively modify his or her behavior in response to punishments (e.g., negative consequences of impulsive actions) and rewards. The development of a positive inner voice supports the process of cognitive inhibition, the ability to suppress distractions, which is also related to behavioral inhibition. The model of thirteen cognitive skills underlying attention and impulse inhibition is provided in Table 2 below.

TABLE 2
13 Cognitive skills underlying attention and impulse inhibition

| Type | Skill | Definition |
|---|---|---|
| Attention | Focused Attention | Arousing one's attention and focusing it on a |
| | | stimulus |
| | Sustained Attention | Maintaining attention on a stimulus for a |
| | | prolonged period |
| | Selective Attention | Directing attention toward a specific stimulus |
| | Alternating Attention | Rapidly shifting one's focus of attention |
| | Divided Attention | Maintaining two or more focuses of attention |
| | | simultaneously |
| Impulse/inhibition | Cognitive Inhibition | Suppressing distraction from irrelevant |
| | | thoughts |
| | Behavioral Inhibition | Suppressing pre-potent or impulsive |
| | | responses |
| | Interference Control | Suppressing distraction from irrelevant |
| | | stimuli |
| | Novelty Inhibition | Suppressing distraction from new stimuli |
| | Motivational Inhibition | Modifying behavior in response to |
| | | punishments or rewards |
| | Self-Regulation | Modifying behavior in response to future |
| | | goals and past outcomes |
| | Inner Voice | Use of an inner monologue to motivate and |
| | | guide actions |
| | Delay of Gratification | Suppressing immediate desire for a greater |
| | | long-term reward |

Each of these thirteen cognitive skills were then aligned to effective challenge tasks, and video game mechanics based on those activities were developed. Additional cognitive skills can be added to expand the skills trained in the virtual learning curriculum.

### Game Design

The game uses an Attention and Impulsivity Model (AIM) to train a user to improve his or her inattention and impulsivity controls (e.g., targeted cognitive skills). The AIM can include several different cognitive process skills for the user to master within those for attention and impulse inhibition and beyond into the other executive functions. A different cognitive skill is taught in each of multiple levels of the game's learning curriculum by the use of corresponding game skill teaching mechanics to the targeted cognitive skills. Levels in the game can include one or more game missions that include various game challenge tasks and goals for the user. Within each mission there are at least two modules, a skill training module and a skill transfer module. Through this design, the skill training module may consist of an adventure narrative in the skill training module, and the transfer module returns to a real world environment within the story line where the skills learned in the game learning curriculum module can be maximized by practice, transferred to real life and reinforced by daily use. In particular embodiments, in order to progress to different missions and levels in the game, success performance measures reflecting the desired skill must be demonstrated in both the skill training module and in the skill transfer module.

The presently described cognitive skill learning curriculum utilizes a feedforward modeling methodology, which enables rapid learning from actual experience in achieving a modeled desired goal emerging within the curriculum. It first requires the user to proactively focus their sustained attention at raised attention state levels to move into an optimized learning zone for rapid cognitive skill(s) development. By the user feeding forward their raised attention state levels to meet emerging and modeled cognitive skill challenge tasks in the oncoming environment, the user utilizes and self-develops whatever cognitive skills to meet the challenge of achieving the desired goal (i.e., modeled) with failure or success. Maximizing ones attention state levels enables the user to rapidly see the desired goal and develop naturally the cognitive skills to meet these oncoming challenge tasks. Recent studies suggest that feedforward learning mechanisms involved in teaching cognitive processes contribute to accelerated and efficient skills learning where feedback learning mechanisms are poor models for learning. Feedforward modeling and rapid learning occurs when knowledge of the desired goal is illustrated (i.e., modeled) in front of the person and used by that person to guide his or her future action or inaction to achieve that desired goal. Thus, a distinctive element of feedforward modeling processes is the ability to envision the future, which engages and drives up a user's attention level for optimizing the learning experience or zone. The cognitive skill training modules can induce the user to feedforward his or her highest attention levels to enable learning (e.g., rapid learning) of specifically targeted cognitive skills that build and strengthen attention circuits in a user's brain. Specifically, by dynamically modeling targeted cognitive skills using virtual challenges (e.g., challenge tasks) with the user's heightened attention state levels, the cognitive skills training exercises target neural circuitry corresponding to supporting those desired cognitive skills of attention and impulse inhibition. The user or first person user can anticipate the rewards offered by the game's narrative (e.g., adventure story) and ultimately bring his or her attention states to the level required to attain those challenge tasks and rewards. As the game (e.g., adventure game) progresses, the game (e.g., the learning curriculum of the challenge tasks) instantly adapts to the user's changing attention state level and/or performance so as not to become too easy or too difficult for the user (e.g., and thus fully engaging the user through their iterative failure, striving and success). In some embodiments, this instant algorithmic adaptability is able to maintain optimal engagement and personalize feedforward modeling for each user and maximal neural stimulation in attention and impulse inhibition-associated circuitry.

Cognitive Skill Training Modules

The Cognitive Skill Training Modules are each comprised of two primary components: a) feedforward of personalized and precisely calibrated attention state levels to accurately define and maximize one's attention state levels to the training, learning and experience of the cognitive skills education, and b) virtual learning curriculum or instructive strategy (pedagogy) of dynamic cognitive attention skill modeling through uniquely defined challenge tasks that directly compare a user's personal cognitive skill performance against ever higher skill levels of those cognitive skills underlying attention and impulse inhibition presented in Tables 1 and 2. The pedagogy of each module sets a limit on the time per training session, minimum rest time between training sessions, and minimum number of training sessions to ensure the building and reinforcing of new brain neuronal circuitry. In one embodiment, the program can be designed for training sessions of 10-60 minutes with regulated rest periods of at least 12 hours, including sleep, to occur between sessions. The training can be designed to progress at the rate, for example, of 3-7 sessions per week over the course of 3-8 weeks until the entire adventure game series (learning curriculum) is completed over an expected total of at least 8 hours (approximately 24 twenty minute sessions) of training sessions. The Cognitive Skill Training Modules are designed to optimize cognitive skill learning by providing a sufficient amount of time to iteratively learn the new skill, tire of the exercise and exhaust the attention circuit, then rest, recover and re-exercise the same neuronal circuit by repetitive skills learning challenges before exhausting the circuit again. This iterative neuronal exercise process of developing one's natural neuroplasticity is known to build and reinforce the brain circuitry specifically attributable to learning. Feedforward modeling, while one of multiple different teaching methodologies, uniquely begins with the user recognizing the necessity of developing his or her ability to raise and maintain his or her attention level to enable success in achieving a targeted challenge tasks and missions within the adventure video game series (i.e., virtual learning curriculum). While a user's attention state levels are heightened, the instructive strategy (e.g., pedagogy) of each of the training modules delivers customized training for each of the 13 cognitive skills presented in Table 2. The combination of the feedforward learning methodology with the dynamically modeled cognitive skills training has demonstrated efficient and rapid cognitive skills learning and transfer to real life environments. For example, after completing the course of the game based learning curriculum series, the user learns to aggregate, recognize and select only relevant information and ignore irrelevant and distracting information and apply their increasing cognitive skills in school, home, work and life.

Feedforward modeling fundamentally consists of being able to recognize the need for a future action or inaction to achieve a goal prior to realization of the consequence of inaction or action and then taking the appropriate action or inaction in order to achieve that future desired goal. In one example, the user recognizes he or she must keep his or her attention state level high in order to move through all training missions, reach the end of the adventure story by successfully meeting the desired goals of the many challenge tasks presented by the learning curriculum. For example, a number of walled barriers that must be circumvented before the user, as a first person avatar within the game, can complete that session. In particular embodiments, to achieve that desired goal the user must instantly recognize when his or her attention state level is about to drop and take personal action to return to a higher level of attention state while moving around the wall barriers. For optimal feedforward modeling, the goal and the difficulty can be precisely personalized to the user's actual attention state levels in the instant of change to maintain optimal engagement and cognitive skill learning.

Personalization of the difficulty challenge of each cognitive skills training is achieved through instant adaptive cognitive skill challenge difficulty adjustments (i.e., dynamic cognitive modeling) based on the user's then targeted cognitive skills performance. Learning of the new and/or increased cognitive skill is best achieved within an appropriate increasing level of challenge tasks for the user while maximizing engagement through optimized combinations of success and failures against oncoming targeted challenge tasks that match the targeted skill to be learned. To maximize the learning and retention of new and increased skills abilities the challenge task difficulty is algorithmically adjusted to a skill level just beyond the maximum of the user's then current skills ability. These dynamic modeling adjustments enable the user to visually see, experience and then move beyond their current skills abilities. To achieve and retain rapid skills learning, the skills learning and transfer modules are designed to adaptively adjust the difficulty of challenge tasks as described in further detail below. The algorithmic model adjusts the challenge tasks dynamically during game play to adapt to the level of difficulty that is calculated to challenge the user to a new or higher level of cognitive skill then last exhibited during the training and/or transfer modules. As the user demonstrates or not targeted cognitive skills proficiency, the algorithmically derived targeted challenge task emerges in front of the user to draw out and engage increasing skill performance levels from whatever performance level demonstrated by the user. This adaptation can be completed in real-time throughout the course of each learning training and transfer session.

Cognitive Skills Transfer Module

Following the user's engagement and performance experience with the cognitive skills training module, the user is led by the story line into the skills transfer module to enable demonstration of the skills learned in the training module. That is, the user independently demonstrates their actual learning and retention of the new skills taught in the earlier training module(s) for later use in life. This cognitive skills transfer module engages the player to apply the same new skill learned but used in a different virtual environment than the one experienced in the skills training module. The environment of the skills transfer module application is designed to more closely align with real life experience outside of the virtual world environment of the game story line. Each skills training module can be within the context of the fantasy adventure story, while the skills transfer modules can be within a more realistic environment, such as a laboratory in a space transport, which is similar in nature to a school, work place or laboratory. The addition of this skills transfer module practice leads to transfer of the newly learned skills to contexts outside of the training, including in home behavior life, work, play and academic performance.

Integration of the Attention and Impulsivity Model (AIM) within the Game Mechanics

Each of the following cognitive skills of the AIM may be integrated into the game based virtual learning curriculum by various teaching mechanics embedded into the adventure story line as described herein:

Attention Maintenance

Attention maintenance (e.g., focused attention and sustained attention) refers to a person's ability to maintain control over his or her level of attention or concentration. This ability enables the person to sustain his or her higher levels of attention states on a stimulus, to know when he or she is becoming less vigilant or begin losing and decreasing his or her level of attention. This skill includes the person's ability to instantly correct or compensate for this loss of attention (i.e., lower state of attention) and make adjustments to his or her attention state levels in real time. The objective of training attention maintenance is to train the user to sustain his or her attention state level at a high learning level for an extended period of time, such as for a full classroom lecture.

In all the skills training and skills transfer modules, the user's attention state levels are continually measured during the session module and is used by the user to feedforward their avatar character into the adventure stories by raising his or her attention state level to communicate, control and dictate the speed at which the avatar character runs or to execute some other comparable speed function in the skills transfer module. This feedforward of a user's attention state levels can be presented in an attention state level from 0% with 0 being the lowest level of attention state to 100% being the highest level of attention state. Directional control is exercised through left, right, up and down arrow keys or swipes with a finger on a computer tablet glass. User movements are correlated to acquisition of correct rewards (both commissions and omissions) or incorrect actions (both commissions and commissions) via tokens and avoidance of obstacles. Such tasks are referred to herein as “collision avoidance and collection challenge tasks.”

Patterns of high, low, and sustained attention state levels are monitored and translated into the character's “Power” ratings. Power ratings can also encompass a measure of challenge task performance (e.g., a composite value integrating attention state levels with challenge task performance). Thus, power ratings provide a visual reward for better performance and high or sustained attention state levels. Power ratings accumulate during the course of a session and can be visually displayed as a power meter. In some embodiments, thresholds on the power meter serve as a gate to progress to the next mission. Awards (e.g., achievements or promotions in rank) can be given to a user as specific Power ratings are reached.

Virtual “distances” are standardized for each session. The time needed to complete a mission provides a composite measure of overall speed and variation during a session. Lower times indicate higher average speeds and/or lower variations in speed within a session. Tokens may be acquired or ignored as they appear in the running path, and obstacles may be avoided by changing lanes, jumping over or sliding under (e.g., collision avoidance and collection challenge tasks). Impulsive movements are identified through the lack of correlation between directional movement and a desired or undesired token or obstacle. These tasks, which test impulse inhibition, are referred to herein as “impulse/inhibition challenge tasks.”

In particular embodiments, the user must maintain a high level of attention state to progress through the game's pedagogy of challenge tasks commensurate with the cognitive skill being taught and the user's success. The user may also encounter challenge tasks during the game where his or her progress through the game is blocked and he or she must raise his or her attention state level in a period of intense or higher focus in order to return to normal progression through the adventure game. In one example, in order to be successful and progress through each of the challenge tasks, the user must learn to control and raise his or her level of attention state, the greater ability to sustain attention state levels at a high level can lead the user avatar to move faster and successful progression through the challenge tasks corresponding to the cognitive process skill being taught in each of the missions and modules. The attention state level scale from 0% to 100% is personalized to the user, and the level of attention state required to unblock game progression changes based on the user's success performance on previous progression blocks.

In particular embodiments, in the skills transfer module, the user must bring his or her attention state level to a high level in order to complete the challenge task. For example, he or she may need to achieve a specific level of attention state in order to interact with the challenge task on the screen. In one example, the user must sustain high levels of attention state in order to complete repetitions of a task. A minimum number of repetitions are required in order to progress to the next skills training module of the game. The user's attention state level (0-100%) is continually measured during the transfer session. Counts for completed challenge tasks are tracked for the entire exercise and independently for subtasks/segments within the skills transfer exercise. Completion rates are measured for the entire exercise and independently for subtasks/segments within the skills transfer exercise.

The user's success performance is presented in the context of the game narrative through speed, Power, and counts of tokens collected and obstacles avoided. In the context of the skills transfer module, the user's performance is presented through average attention state levels, counts of completed challenge tasks, and rates of completion for subtasks/segments. Success performance is rewarded through recognition (status, tokens, and awards) consistent with the game pedagogy. The user's skill assessment is presented as a set of time-series charts for speed, power, and accuracy over the course of each game session and aggregate charts for the skills transfer segments. Overall skill performance is condensed into a single score combining weighted attention and correctly completed challenge tasks in both the skills learning and skills transfer segments. Skills performance reporting may be provided to medical professionals, teachers, parents, users or other third parties.

Selective Attention

Selective attention (e.g., selective attention and interference control) refers to a person's ability to process or focus his or her attention on a specific stimulus that is relevant to the person's goals and to ignore irrelevant stimuli. The objective of training selective attention is to train the user to complete all parts of a challenge task in a distracting environment, such as completing class work at school homework at home or an employment task.

In particular embodiments, in the skills training module, when faced with a group of challenge task relevant stimuli, the user must identify if a target stimulus is present within that group and select the correct target while ignoring (i.e., not selecting) non-target stimuli. The user achieves future success in his or her adventure for selecting the correct target and ignoring the incorrect non-targets. The user is pushed back from his or her success for selecting incorrect non-targets or ignoring correct targets and the user's progression towards a future goal can be hampered. In one example, to complete a skills training module, the user must successfully attend to and select the identified targets (the goal), while ignoring both distractors in the environment and non-target stimuli that do not further the goals of that module. The number of stimuli in the group, the specificity of the targets, and the response window (i.e., the time the user has to respond to the group) can be dynamically adjusted based on previous performance with other groups of stimuli.

The user's actions are continually scored for correct and incorrect response to challenge tasks to learn targeted cognitive skills. Patterns of correct and incorrect responses to stimuli are monitored. The user recognizes that a continuous sequence, or stream, of correct responses are rewarded with visual indications of successful performance (e.g., through a power meter). Patterns of sustained attention state levels are monitored. Higher levels and sustained state levels are rewarded by visual indications of successful performance (e.g., through a power meter). Power can accumulate during the course of each module. Awards (e.g., achievements and/or rank promotions) can be given as specific power levels are reached. For example, stars may be awarded in-game when a predetermined “power level” is achieved. These can be seen on the power meter when a mission begins, and an animation and/or sound indicates when the predetermined power level is reached and the star is awarded. Achievements and rank promotions can be awarded, for example, based on average attention state level and/or challenge task performance (e.g., accuracy in target response challenge tasks and/or collection and collision avoidance challenge tasks). In some embodiments, achievements and rank promotions are awarded at the end of the mission.

In the skills transfer module, the user can be presented with a group of challenge tasks commensurate with the previous skills training module. In one example, the user must selectively pay attention to targets, which can further his or her goal of increasing the score, and selectively ignore non-targets. In another example, the user must achieve a minimum success score in order to advance to the next training segment. The score can increase for correct selections of targets and decrease for incorrect selections of non-targets. The user's actions are continually tracked for correct and incorrect response to challenge tasks which build and strengthen new brain circuits.

The user's success performance is presented as described in the previous section. Skills performance reporting may be provided to medical professionals, teachers, parents, users or other third parties.

Alternating Attention

Alternating attention (e.g., alternating attention and divided attention) refers to a person's mental flexibility in rapidly shifting attention from one task to another. The objective of skills training alternating attention is to train the user to follow instructions and execute multiple tasks, such as get dressed and then return downstairs.

In the skills training module, the user can be presented with two different challenge tasks to complete. In one example, the user must keep the instructions for two challenge tasks in mind and rapidly shift between each challenge task. For example, instructions can provided in the form of target rules, e.g., rules that identify an object as a target or non-target. At any given moment during the training module, the user can be performing at least one of the two challenge tasks. After certain intervals, the user may need to change which challenge task he or she is performing or, for example, the target rules may change. Failure to complete the correct task successfully (i.e., to switch to the current task or current set of target rules) can hamper the user's progression through all of the challenge tasks of the game that lie ahead. The rate of switching, and the predictability of when a switch between tasks can occur, can be adjusted based on previous performance on each challenge task and on switching between the challenge tasks.

In the skills transfer module, the user can have two (or more) different challenge tasks presented to them. In one example, the user must keep the instructions for the challenge tasks in mind, notice the indication to shift challenge tasks, and successfully shift to the other challenge task. The target task for completion can change intermittently, with the change indicated by a change in target challenge task. The user may need to achieve a minimum success score by completing multiple repetitions of the two challenge tasks.

The user's success performance is presented as described in the previous sections. Skills performance reporting may be provided to medical professionals, teachers, parents, users or other third parties.

Behavioral Inhibition

Behavioral inhibition (e.g., behavioral inhibition, motivational inhibition, and cognitive inhibition) refers to inhibiting or suppressing a pre-potent learned response when that response would be inappropriate in the given context. The objective of training behavioral inhibition is to train the user to act appropriately for different contexts and inhibit inappropriate responses, such as behaving quietly at a doctor's office.

In the skills training module, the user can be presented with a series of challenge tasks that teach the targeted cognitive process. The majority of the challenge tasks (greater than or equal to 50%) can be targets that the user should select to move forward in the adventure game. In particular embodiments, the remaining challenge tasks can be non-targets and the user must inhibit responses to these tasks or challenges. The user will have learned a primary response to select the correct stimuli, since the majority of stimuli will be the correct targets. In one example, the user must inhibit this learned behavior when presented with a non-target. The ratio of targets to non-targets and the user's response window can be dynamically adjusted based on previous success performance with each challenge task.

In the skill transfer module, the user can be presented with a set of stimuli or game mechanics to demonstrate skills retention, the majority of mechanics can require a specific action. A minority of the challenge tasks can require that action not be taken (i.e., to be inhibited). For a subset of the stimuli, the user may need to inhibit the primary action he or she has been completing, since it is inappropriate for that stimulus.

The user's success performance is presented as described in the previous sections. Skills performance reporting may be provided to medical professionals, teachers, parents, users or other third parties.

Novelty Inhibition

Novelty inhibition refers to the ability to recognize when a novel stimulus is irrelevant, and to subsequently ignore it and return to the person's current task or goal. The objective of training novelty inhibition is to train the user to be able to complete learned tasks when confronted with novel situations or changes in environment that are not relevant to completing the task, such as behaving properly during the first day of a new school year.

In the skills training module, the user can be presented with distractions or environments he or she has not previously encountered in the training modules. In particular embodiments, the user may be completing a previously trained skill and must ignore the novelty changes. In one example, the user may only be asked to complete challenge tasks, which he or she has successfully completed earlier in the game, but he or she must ignore irrelevant, but novelty changes to the environment in which he or she is completing the task. The difficulty can be adjusted in accordance with the other cognitive skills the user is performing. By changing the difficulty of the challenge task, the difficulty of inhibiting responses to novelty, irrelevant distractors can also change.

In the skills transfer module, the user can be completing a previously mastered skills transfer task. Novelty distractions can be introduced, such as additional irrelevant distractions or changes in environment. The user may experience changes during the challenge task that are not relevant to the user's ability to complete the task. In particular embodiments, to maintain a high level of success performance and therefore progress, the user must ignore these novelty occurrences.

The user's success performance is presented as described in the previous sections. Skills performance reporting may be provided to medical professionals, teachers, parents, users or other third parties.

Delay of Gratification

Delay of gratification refers to the ability to inhibit or suppress an action that would result in an immediate reward in order to gain a larger reward later. The objective of training delay of gratification is to train the user to be able to forego an immediate reward in order to achieve a greater reward later, such as doing homework first instead of watching television or playing to receive better grades and more time to play after completing homework.

In the skills training module, the user may have opportunities to either receive a small immediate reward, such as positive feedback at the moment, or to take actions that lead to greater progress in the game story line. In particular embodiments, to successfully progress through the game, the user must choose actions that support overall progress and success rather than actions that lead to small, immediate rewards. As the difficulty of individual challenge tasks change, the complexity of the user's decisions and the need to delay reward can likewise change. The user's actions are continually measured for correct and incorrect response to stimuli while challenged by token acquisition and obstacle avoidance in close proximity. The correct sequence of response (prioritization), in addition to correct responses to stimuli, are used to generate a composite of successful skill performance.

In the skills transfer module, the user may have opportunities to either receive a small immediate reward, such as a positive reward or a small increase in score at the moment, or to take actions that lead to greater increases in the success score overall. In particular embodiments, to successfully progress through skills learning and retention modules of the game, the user must choose actions that increase the overall success score high enough to meet the minimum score level for advancement, which can require a larger increase in score than is possible with only the small increases. The user's actions are continually measured for correct sequence of response (prioritization) in addition to correct responses to stimuli.

The user's success performance is presented as described in the previous sections. Skills performance reporting may be provided to medical professionals, teachers, parents, users or other third parties.

Inner Voice

Inner voice refers to the ability to use one's own internal self-talk to provide analysis, reasoning, motivation, and guidance through solving a problem or completing a task. The objective of training inner voice is to train the user to be able to self-motivate completion of tasks, including tasks with multiple steps, such as completing a math word problem, reading a chapter or navigating through a city by car.

In the skills training module, the user can be presented with both a peer and mentor characters to begin to model inner voice for questioning and answering challenges. Throughout the pedagogy of challenge tasks of the game, the peer can provide guidance to the user avatar that can facilitate skills development and learning. As the game progresses, the peer character may provide less skill guidance relying on the user to independently develop his/her own self guidance, confidence, esteem as their own inner voice. The guidance provided by the peer and the user avatar model demonstrate appropriate development of inner voice. The reduction of guidance from the peer figures promotes the user's internalizing the guidance and confidence and increasing his or her self-talk for the purposes of self-motivation and problem solving. The amount of guidance provided by the peer figures can be adjusted based on current level of success performance and/or the current point of progression through the adventure game. For example, peer guidance can be triggered by performance, wherein low rates of power accrual generate notifications (text, graphics, sound) of encouragement and higher rates of power accrual generate notifications of accomplishment. Notifications of accomplishment can occur less frequently with higher levels of success performance.

In the skills transfer module, the user can be presented with a wise mentor. Throughout the course of the game's skills transfer modules, this mentor can provide the environment and self challenge to the user avatar to independently adapt the skills learned to a new application. This demonstration (or not) of the newly learned skills effectively transfers the skills learned by the user in the skills training module to real life. As the adventure game progresses, less guidance may come from the mentor and the user can provide more self-challenge, wisdom, and experience to demonstrate their inner voice. The self challenge provided by the wise mentor and the user demonstrate appropriate development of inner voice. The reduction of self challenge from the mentor promotes the user internalizing the wisdom and increasing his or her self-talk for the purposes of self-motivation and problem solving

The user's success performance is presented as described in the previous sections. Skills performance reporting may be provided to medical professionals, teachers, parents, users or other third parties.

Self-Regulation

Self-regulation refers to the ability to remain goal-oriented, motivated, and organized while constantly monitoring and assessing one's behavior. The objective of training self-regulation is to train the user to be able to prioritize different tasks and create plans for completing those tasks, such as completing homework for different users.

In the skill training module, the user can be presented with multiple tasks, pertaining to the skill being trained or being secondary rewarded tasks. In one example, the user must prioritize the challenge task and plan his or her actions to optimize the amount of success rewards achieved. The user may need to choose his or her actions to achieve his or her goal (remain goal-oriented) and evaluate whether his or her action plan was successful or if he or she need a different choice of actions to get a higher level of success rewards and progress more quickly through the adventure game. As the difficulty of individual skill tasks change, the complexity of the decisions and necessary plans for success can likewise emerge and change based on actions taken. The user's actions are continually measured for correct and incorrect response to stimuli while simultaneously challenged by token acquisitions and obstacle avoidance. The correct selection of the stimuli over token acquisition and obstacle avoidance, and the correct response to stimuli are used to generate a composite of skill success performance.

In the skills transfer module, the user may need to evaluate his or her performance as it pertains to the success score level achieved. If he or she does not meet the minimum score level, the user may need to reevaluate how he or she performed and improve success performance before being able to move to the next skill training segment. In one example, the user must evaluate his or her action plan and success performance in relation to the goal of achieving a higher success score. The user's actions are continually measured for correct selection and response to stimuli over other simultaneously presented tasks.

The user's success performance is presented as described in the previous sections. Skills performance reporting may be provided to medical professionals, teachers, parents, users or other third parties.

### EEG Data Collection

The invention features methods and systems that utilize EEG (electroencephalogic brain waive activity) data. The EEG data can be collected, for example, using an electrode system in the form of a headset. Headsets suitable for use in the invention include those described, for example, in U.S. Ser. No. 14/179,416, incorporated herein by reference. The International 10-20 System provides for standardized electrode locations, and recently higher density systems have been developed (sometimes called the 10-10 System). The headsets of the invention can be designed to (i) intuitively and conveniently place electrical sensors at positions AF3 and AF4 (as well as a ground electrode, which optionally is placed at the mastoid) of the 10-10 system on the forehead of a child (i.e., without significant training in how to wear the headset), (ii) account for the variability in head size among children of different ages, and (iii) be comfortable to wear. For example, the headsets can be sized and configured to accommodate a range of head sizes.

The headsets contain electrical sensors that measure EEG signals that are processed by an external computer. The electrical sensors can include one or more electrodes for measuring EEG signals of a user. The electrodes can be dry electrodes or wet electrodes (i.e., a dry electrode can obtain a signal without a conductive and typically wet material between the electrode and the user's skin, and a wet material does require such a conductive material). The electrical sensor can include a dry electrode, such as a dry fabric electrode. Fabric electrodes suitable for use in the methods and systems of the invention include those described in U.S. Patent Pub. No. 20090112077, incorporated herein by reference. The electrical sensors can contain padding to aid in the comfort of the user and also aid in adjustability and improving skin contact.

The invention features the collection of EEG brain activity data, which is amplified, converted and communicated to a computer for processing during a game session to produce a measure of attention state levels in the user scaled 0%-100%. Others have demonstrated the use of, EEG-data from various frequency bands of a user's brain signal activity that can be used to determine the relative attention state level in a user using a theta to beta ratio. That is, relatively greater beta (approximately 16-32 Hz) activity has been observed in vigilant states, whereas alpha (approximately 8-16 Hz) activity predominates in alert but less mentally busy states, and theta (approximately 4-8 Hz) activity rises as attention lapses (Streitberg et al., Neuropsychobiology 17 (1987): 105-117). Methods of gathering and interpreting EEG data for monitoring attention levels are known in the art and described, for example, in U.S. Pat. No. 8,862,581; U.S. Patent Publication Nos. US20120108997 A1, US20100145214 A1, US20110289030 A1, and US20130331727 A1; U.S. provisional Ser. Nos. 62/172,601, and 62/199,749; and International Application No. PCT/US2016/044828, each of which is incorporated herein by reference. In addition to distinct frequency bands, EEG signals can be acquired at distinct recording sites at the brain. For example, the voltage between the AF3 and AF4 electrodes reflects electrical activity in the dorsal anterior cingulate cortex. In studies utilizing functional magnetic resonance imaging (fMRI) has been observed that the dorsal anterior cingulate cortex becomes active when attention lapses (Uddin et al., Journal of Neuroscience Methods 169 (2008): 249-254).

### EXAMPLES

The following examples are put forth so as to provide those of ordinary skill in the art with a complete disclosure and description of how the methods and systems claimed herein are performed and evaluated, and are intended to be purely exemplary of the invention and are not intended to limit the scope of what the inventors regard as their invention.

### Example 1. Attention Maintenance Game Elements

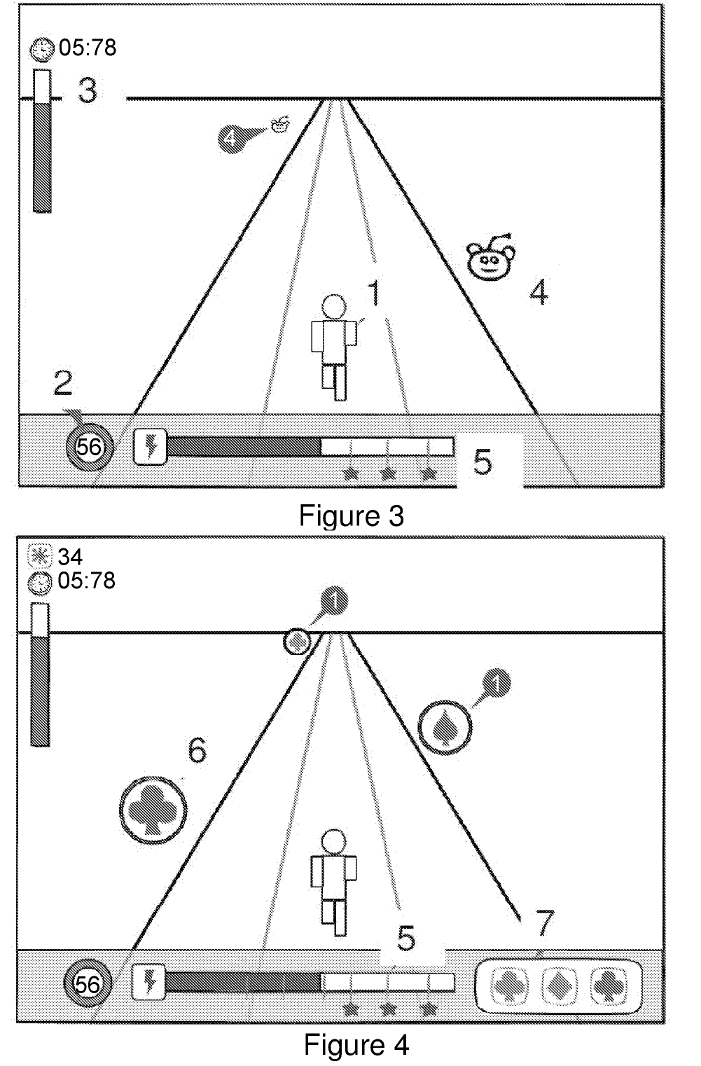

An example of the kind of environment and tasks in which “Attention Maintenance” skills are trained and assessed is provided in FIG. 3. The user avatar 1 runs forward along a path at a speed derived from the user's level of attention states as measured and communicated through the EEG headset to the computer or tablet 2. A timer 3 records the duration of the run and a progress meter shows the distance to the end of the path. In this example, the user must utilize Self-Regulation to persevere until the end of the path and Attention Maintenance to achieve a faster completion time. Distracting objects and creatures 4 appear in the path, and the user must ignore the distractions to sustain his or her raised attention state level and speed through that segment. A “Power Meter” 5 measures the user's Attention Maintenance skill level; the meter fills at a rate that reflects a composite between attention state level and challenge task performance, (e.g., it fills faster when levels of attention states are maintained over a longer time period). A minimum success score on the Power Meter is required to move on to the next mission.

### Example 2. Behavioral Inhibition Game Elements

An example of the type of environment and tasks in which “Behavioral Inhibition” skills are trained and assessed is provided in FIG. 4. Objects with specific visual characteristics 6 appear above the path and move toward the user avatar. A list of target objects 7 appears in the user's display. In this example, the user must tap on an object when its visual characteristics match one of the targets, but utilize Behavioral Inhibition to let objects pass that do not match. In some cases, the visual characteristics of an object are not visible to the user, and only become visible when the user approaches the object. In this case, when a user selects the object prior to its revealing its visual characteristic, that response is identified as an impulsive response. The Power Meter 5 in this environment measures the user's Behavioral Inhibition skill level; the meter fills faster when the user makes correct actions on the object, and when the user makes many successive correct actions. A minimum success score on the Power Meter is required to move on to the next mission.

### Example 3. Selective Attention Game Elements

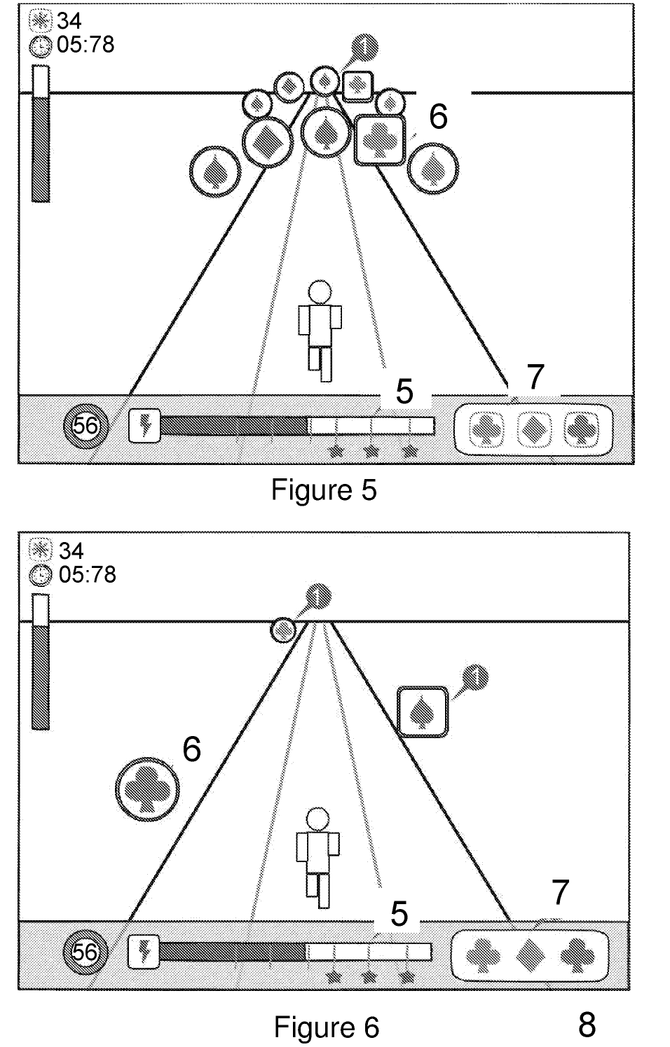

An example of the type of environment and challenge tasks in which “Selective Attention” skills are trained and assessed is provided in FIG. 5. Groups of objects with specific visual characteristics 6 appear above the path and move toward the user avatar. A list of target objects 7 appears in the user's display. In this example, the user must utilize Selective Attention to identify the object within the group that matches one of the targets, and select that object. The Power Meter 5 in this environment measures the user's Selective Attention skill level; the meter fills faster when the user makes correct actions on the object groups, and when the user makes many successive correct actions. A minimum success score on the Power Meter is required to move on to the next mission.

### Example 4. Alternating Attention Game Elements

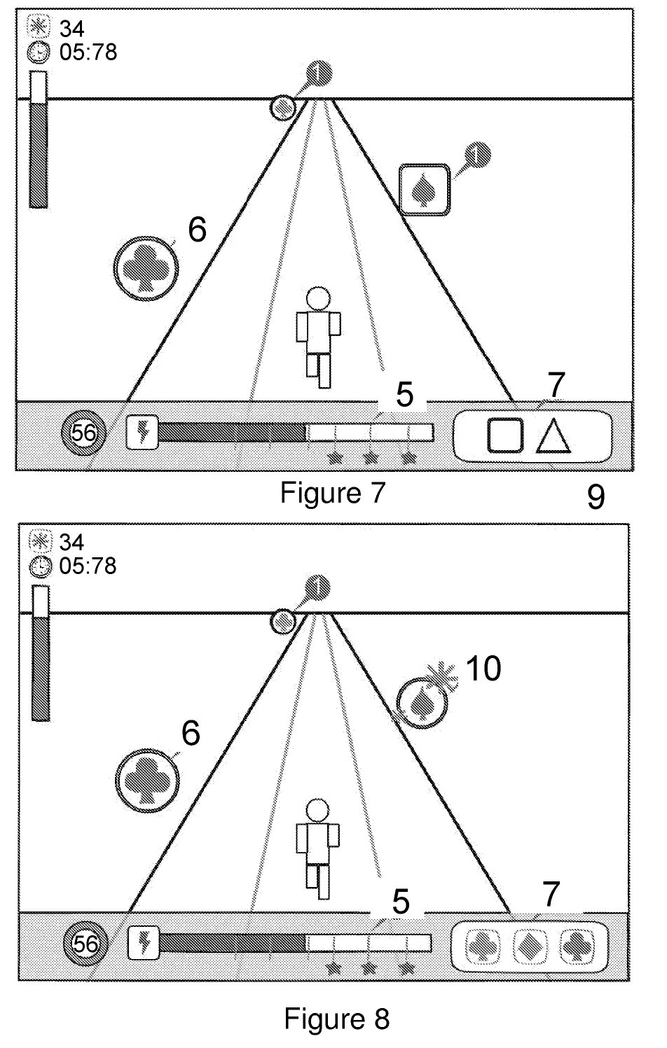

Examples of the type of environments and tasks in which “Alternating Attention” skills are trained and assessed are provided in FIGS. 6 and 7. Groups of objects with specific visual characteristics 6 appear above the path and move toward the user avatar. A list of target objects appears in the user's display, sometimes showing one visual parameter such as a symbol 7 and other times showing a different visual parameter such as a shape 8. In this example, the user must identify the objects that match the parameter shown, and utilize Alternating Attention to select objects that match the relevant parameter even as the relevant parameter changes rapidly. The Power Meter 5 in this environment measures the user's Alternating Attention skill level; the meter fills faster when the user makes correct actions on the objects, and when the user makes many successive correct actions. A minimum success score on the Power Meter is required to move on to the next mission.

### Example 5. Novelty Inhibition Game Elements

An example of the type of environment and tasks in which “Novelty Inhibition” skills are trained and assessed is provided in FIG. 8. Objects with specific visual characteristics 6 appear above the path and move toward the user avatar. Some of these objects feature visual highlighting details 10 that tempt the user to select the object. A list of target objects 7 appears in the user's display. In this example, the user must tap on an object when its visual characteristics match one of the targets, but utilize Novelty Inhibition to suppress the user's natural response to the visual highlighting. The Power Meter 5 in this environment measures the user's Novelty Inhibition skill level; the meter fills faster when the user makes correct actions on the object, and when the user makes many successive correct actions. A minimum success score on the Power Meter is required to move on to the next mission.

### Example 6. Inner Voice Game Elements

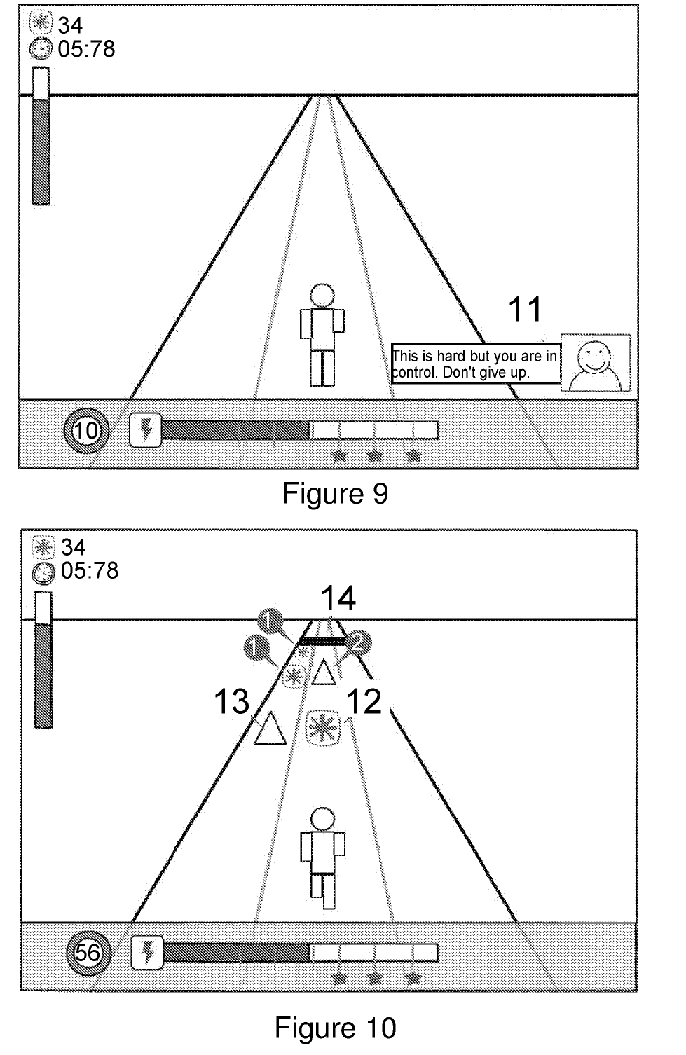

An example of the type of environment and tasks in which “Inner Voice” skills are trained and assessed is provided in FIG. 9. Various user performance thresholds trigger an intervention from a non-user character 11 who provides wisdom and self-esteem encouragement via voice-over dialog. As the game progresses, the user avatar character gradually voices the encouragement to him or herself, modeling for the user the acquisition of an inner monologue to motivate action.

### Example 7. Self-Regulation Game Elements

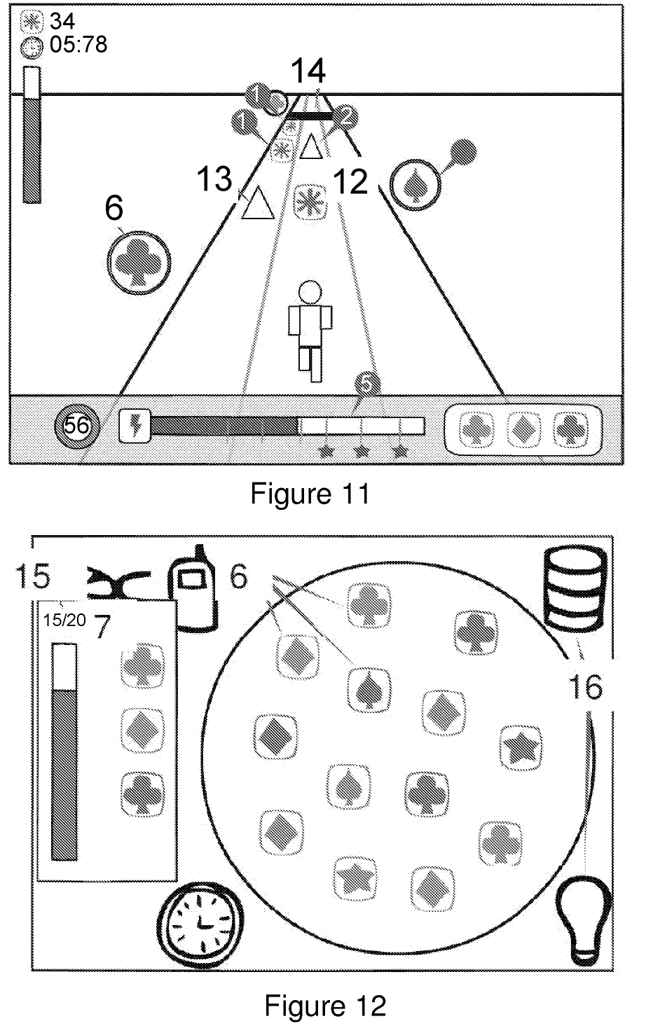

Examples of the type of environments and tasks in which “Self-Regulation” and “Delay of Gratification” skills are trained and assessed are provided in FIGS. 10 and 11. Tokens 12 appear in the path. In this example, the user avatar must run through a token to collect it. Small Obstacles 13 and Large Obstacles 14 force the user to dodge, jump, or duck along the path. If a user avatar collides with an obstacle, the user will lose one of the tokens he or she has collected. In this example, the user must utilize Self-Regulation to collect the maximum number of tokens; he or she must look ahead on the path and plan the user avatar's movements to most efficiently collect tokens and avoid obstacles. In some cases, the user may need to utilize Delay of Gratification to delay the immediate gratification from collecting a token when moving into the token's path would put them at risk for colliding with an obstacle. In environments where objects 6 appear along the path, the user must further utilize Self-Regulation to collect the maximum number of tokens while selecting the maximum number of correct targets, again by looking ahead on the path and planning his or her approach. In some cases, the user may further utilize Delay of Gratification to delay the immediate gratification from collecting a token or avoiding an obstacle in order to select a correct target before it passes from view, as selection of targets is what determines the fill rate of the Power Meter and the ability of the user to progress to the next mission.

### Example 8. Skill Transfer Game Elements

An example of the type of environment and tasks in which the user learns to transfer skills trained in one part of the game to another context is provided in FIG. 12. In this case, the Selective Attention skill is trained with modified game mechanics and in an alternative visual context from that depicted in FIG. 5. In a 2D presentation, multiple objects with distinct visual characteristics 6 appear all at once in an area of the screen and stay visible for a few seconds. A list of target objects 7 appears in the user's display. In this example, the user must utilize Selective Attention to identify the objects within the area that match one of the targets, and select those objects. If the user selects an object that does not match the list, all of the other objects disappear. Later, the user must raise his or her attention state level above a threshold in order to collect any objects selected. A counter 15 records the number of correct objects collected and represents a success measurement of the user's Selective Attention skill.

### Example 9. Procedures for Studying Effectiveness of the Training System

The training system can be evaluated for effectiveness in children suffering from ADHD. During an initial consultation, the clinician can evaluate the user's inattentive symptoms using an ADHD-RS and overall inattentive severity using a Clinical Global Impression-Severity (CGI-S) scale to ensure eligibility and monitor individual changes pre and post training.

Training Duration and Frequency

The first training session can be completed in 20-25 minutes. The second training session and all subsequent training sessions can be completed in 20-30 minutes. Users can train 3-7 times per week for 3-8 weeks. The skills transfer module can be administered to the users after each skills training module. As the training progresses, game skills development progresses through one or more of the skills detailed in the AIM. Users can move to the higher skill levels as they successfully complete previous session objectives in the form gating challenges for both the skills training and skills transfer modules.

Training Session Procedures

The training system can include the game on a PC laptop (or computer, tablet or personal electronic device) and an EEG headband. During each game session users will play the game (skill training). As the users play the game, their EEG waves can be recorded simultaneously via the EEG sensors embedded in the headband. The EEG waves can be used to quantify the subjects' level of attention states in real time, which quantification into attention state levels scaled 0%-100% can ultimately control the speed of the avatar in the adventure game. The higher the attention state level the faster the avatar character can move through the missions toward adventure completion. Game sessions can last 20-30 minutes. The game can consist of attention and inhibition skill development based on the AIM. The initial training can be focused and sustained attention, challenging the user to move the character quickly while interacting with audio and distractors. Additional skills can be introduced in subsequent sessions as the user progresses through skill training and transfer modules. Users can be rewarded with points for exhibiting higher and/or sustained attention state levels and for their correct response to selection and rejection stimuli of varying priority. The level of difficulty dynamically can change throughout gameplay based on the user's ability.

Transfer modules can be played by each user to demonstrate skill retention for transfer to real life applications. This skills transfer exercise can contain the same skill introduced in the training module using a different context such as environment, mechanic, and scoring. Skills in the training and transfer modules can be matched so the newly learned attention and impulse inhibition skills would be exercised to demonstrate skill(s) retention. The user can experience improvements in cognitive skills following training using, for example, any of the assessment methods described below, or other methods known in the art for assessing cognitive skills.

### Example 10. Identification of Impulsive Responses and Interventions to Reduce User Impulsivity

The virtual learning curriculum of the invention can include programs and methods for identifying impulsive responses by a user during the performance of the game based curriculum. These programs and methods include incorporation of a delay between the initial presentation of a stimulus and the prompt for the user's response. Challenge tasks that train a user's impulse/inhibition are referred to as impulse/inhibition challenge tasks. As part of an impulse/inhibition challenge task, a mission may provide specific types of stimuli to which the user is to respond, while ignoring the remainder of a full set of stimuli. A user's responses to the stimuli during the delay and before the response prompt (e.g., before the user receives the information needed to correctly respond) can be classified as impulsive. Impulsive responses also include responses in the absence of stimuli. For example, if the mission's path is devoid of challenge tasks and the user responds, those responses can be classified as impulsive.

In the transfer module, an impulsive response can be defined as a response that occurs after a complete response has already been made. For example, a program may include a delay for a duration of time after a user has completed his or her response. In this case, if the user responds during this delay, the response is classified as impulsive.

The training systems of the invention can include programs and methods for reducing impulsivity by the user. These include presenting positive audio-visual sound effects and game rewards as a consequence for correct responses, while presenting negative audio-visual sound effects and restricting game rewards as a consequence for impulsive responses. Additionally, adaptive voice-over feedback from a peer character provides a reminder of the correct course of action or inaction and communication of memorable strategies for correctly applying the relevant cognitive skill. Negative audio-visual reinforcement can be accompanied by a loss of points, which may correspond to reduced success in achieving oncoming challenge tasks These methods also include the requirement of a minimum threshold of non-impulsive responses before the user can progress to the next game mission.

### Example 11. Identification of User Frustration and Interventions to Reduce User Frustration

The training and transfer modules of the invention can include programs and methods for identifying frustration or anxiety by a user during the performance of the game based learning curriculum by modeling improved skills and behaviors. These programs and methods include maintaining a running count or calculation of the number of serial or total incorrect responses to game challenges (e.g., challenge tasks), and triggering changes to the curriculum when this number exceeds a threshold (e.g., a reasonable margin or frequency of error). For example, a serial threshold might be set to three incorrect responses in a row. Alternatively, a running count threshold might be set to 3 out of the last 5 responses were incorrect (e.g., incorrectly selected or incorrectly rejected). Alternatively, a total threshold might be set to 10 total incorrect responses.

These programs and methods include maintaining a running count or calculation of the number of serial or total incorrect responses to game challenges (e.g., challenge tasks), and triggering consequential negative impacts on a user's overall success.

The learning curriculum of the invention can include programs and methods for reducing frustration by the user. These include immediately lowering the difficulty of game challenges (e.g., challenge tasks) when the number or running count of serial incorrect responses exceeds the threshold (e.g., a reasonable margin or frequency of error), and lowering the difficulty even more precipitously when the user continues to make incorrect responses after the initial lowering. In many cases, this lowering of the game difficulty in response to player frustration is accompanied by dynamic voice-over commentary from the peer character that provides reassurance and/or simple strategies for moderating emotional responses to feelings of frustration.

### Example 12. A Nonpharmacological Intervention for the Treatment of ADHD Children

A clinical study of a feedforward modeling (FFM) system was carried out. Detailed discussion of the methods and results of the study are provided herein.

Methods

This study implemented a randomized, controlled, parallel design comparing this FFM with a non-pharmacological community care intervention. Improvements were measured on parent- and clinician-rated scales of ADHD symptomatology and on academic performance tests completed by the participant. Participants were followed for 3 months after training.

a. Participants

The study took place at three clinical sites. Each site was overseen by one training coordinator (TC) and one clinical investigator. Participants were recruited via clinician recommendations as well as print and web-based advertisements. Interested candidates scheduled initial consultations with one of the study clinicians to determine eligibility and assess the severity of ADHD symptoms.

To participate in the study, participants needed to be children between the ages of 8 and 12, receive an official ADHD diagnosis according to Diagnostic and Statistical Manual of Mental Disorders (DSM) criteria by one of the study clinicians and score a 14 or more on the Inattention sub scale of the clinician-rated ADHD-Rating Scale (ADHD-RS), indicating mild-to-moderate inattentive symptoms (Wigal et al., Journal of Attention Disorders, 10(2006): 92-111). Study clinicians confirmed an ADHD diagnosis according to Diagnostic and Statistical Manual of Mental Disorders (DSM-IV. American Psychiatric Association, 2000; Goodman et al., 17 (2010)) criteria with all participants and also completed a clinician rated ADHD-RS.

Non-eligible children were those on medication for ADHD or comorbid psychiatric conditions. Children with sensorineural deficits (blindness or deafness) or known developmental delays as defined as an IQ of 70 or below were also non-eligible. Children with a medical history of epileptic seizures, traumatic brain injury, stroke, central nervous system tumor or lesion, cerebral hypoxia, skull fracture, or encephalitis were also excluded from the study.

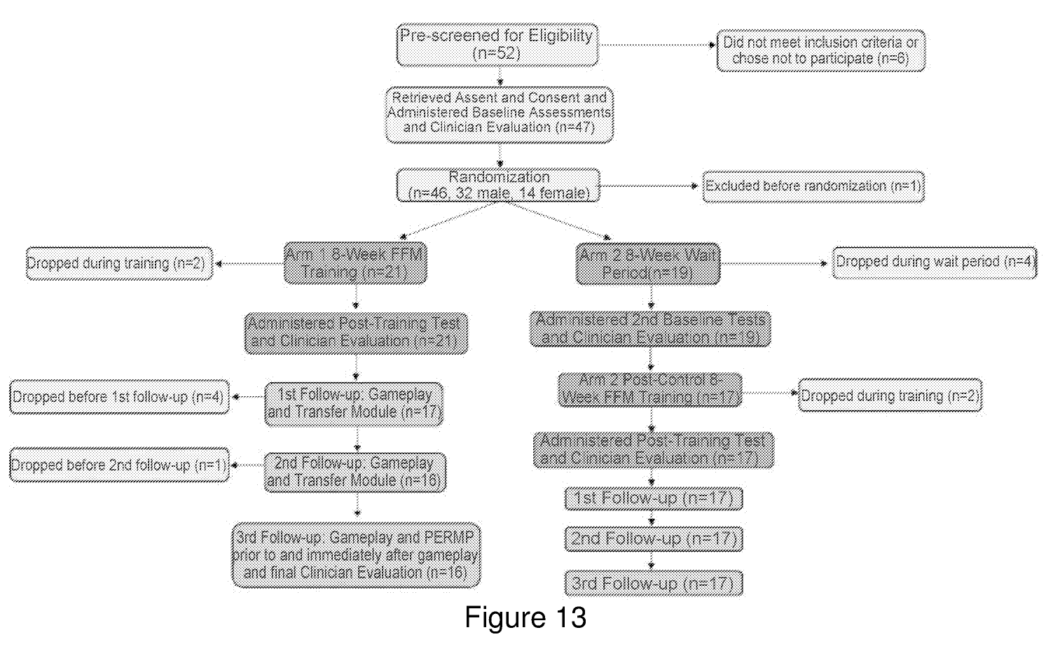

Forty-seven children consented to participate in the current study. Forty-six children were randomized because one participant was falsely deemed eligible during the clinician evaluation and then excluded prior to randomization (32 male, 14 female, M=9.57, SD=1.34). FIG. 13 provides an overview of enrollment and dropouts throughout the course of the study. Dropouts occurred due to scheduling conflicts or no longer meeting the inclusion criteria (e.g., by beginning a medication treatment regimen for a comorbidity).

b. Procedures

During an initial consultation, the clinician evaluated the participants inattentive symptoms using an ADHD-RS and overall inattentive severity using a Clinical Global Impression-Severity (CGI-S) scale to ensure eligibility. At this time, clinicians also spoke with interested parents about all of the non-pharmaceutical options available to improve their children's behavior and attention levels. They reviewed the study and obtained written informed consent from the parent and written assent from the child.

After the clinician consultation, participants completed a baseline assessment visit, during which the participating child completed tests of academic achievement and performance while the parent filled out the ADHD-RS Home Version about their child's behavior. At two of the three sites, participants were also assessed using the Quotient® ADHD System. At the end of this session, participants were randomly assigned to one of two groups. Randomization was stratified by site so that there were approximately equal numbers of participants in Group 1 and Group 2 at each site. Group 1 immediately received the 8-week training using the FFM system application, while Group 2 acted as the control group and received 8 weeks of conventional non-pharmaceutical care. These standard non-pharmacological intervention options included cognitive behavioral therapy once a week, therapeutic tutoring once a week, three to four parent coaching sessions, or minimal or no structured intervention with periodic clinician visits to monitor symptoms. After the first 8 weeks were completed, Group 2 met with the clinician to reassess symptoms and completed another baseline assessment. After these visits were completed, the control group participants received the FFM training for 8 weeks to ensure that both groups would see the same amount of improvement with FFM training and served as an incentive for the control group.

Once the active group's training with FFM system was completed (Week 8 for Group 1 and Week 16 for Group 2), participants completed clinician visits and assessment sessions using the same assessments as baseline. Group 2 did not complete the Woodcock-Johnson Assessment at Week 16 as there were only two versions, which were used for prior assessments.

Participants returned for three monthly booster sessions that involved one game play session, including a skills transfer module. At the third monthly follow-up, in lieu of the skills transfer module, the participant completed the Permanent Product Measure of Performance (PERMP) before and after the gameplay. The booster sessions were used to evaluate whether participants remembered how to use the game after no longer playing on a regular basis. Parents were also asked to complete the ADHD-RS. At the end of the 3 months of follow-up, participants had a final clinician visit to assess their symptom severity.

c. Behavioral Measures

i. ADHD-RS

The ADHD-RS is an 18-item scale that assesses symptom severity associated with ADHD. The clinician completes it based on their interactions and observations of the participant and discussion with parents. Parents were also asked to complete the Home Version of the ADHD-RS, which has been validated for independent completion by parents (DuPaul et al., ADHD Rating Scale-IV: Checklists, norms, and clinical interpretation, 1998). The ADHD-RS is comprised of an Inattention subscale and a Hyperactivity/Impulsivity subscale, as well as a Combined score, calculated as the sum of the two subscale scores. Each of the 18 items are rated on a 4-point scale (0=never/rarely, 1=sometimes, 2=often, 3=very often) and correspond to the diagnostic criteria found in the DSM-IV.

ii. CGI Scale.

The CGI is comprised of two companion one item measures that assess the severity of functioning and psychopathology before and after initiation of an intervention. The clinician considers his or her knowledge of the patient's medical history, behavior, psychosocial circumstances, symptom severity, and the impact that these symptoms have had on his or her ability to function to score the participant (Guy, Clinical Global Impressions (CGI) Scale, 2000). On the CGI-S, participants were rated on a 7-point scale (1=normal; 2=borderline mentally ill; 3=mildly ill; 4=moderately ill; 5=markedly ill; 6=severely ill; 7=extremely ill). The Clinical Global Impression-Improvement (CGI-I) scale assesses how much the participant had improved or worsened since the initial visit. The CGI-I scale was also rated on a 7-point scale: 1=very much improved, 2=much improved, 3=minimally improved, 4=no change, 5=minimally worse, 6=much worse, or 7=very much worse (Goodman et al., 17(2010): 44-52; Wigal et al., Child and Adolescent Psychiatry and Mental Health 3(2009)).

iii. Quotient® ADHD System.

Using a motion tracking system, a forehead reflector, a liquid crystal display (LCD) screen showing visual stimuli, and a keyboard used to respond to stimuli, the Quotient® (Pearson Education, Inc., Westford, Mass.) is a diagnostic tool cleared for marketing by the FDA to provide objective measures of the symptoms of ADHD to aid a clinician in diagnosis (Sumner, The ADHD Report, 18(2010): 6-9). The Quotient® measures attention by requiring the participant to perform a task where he or she is instructed to hit the space bar when the target (8-point star) appears on the screen and to withhold response when the non-target (5-point star) appears on the screen. The system creates a composite score based on the participants levels of hyperactivity, impulsivity, and attention compared with norms based on age, grade, and gender. The Quotient® was only administered at two out of the three clinical sites due to availability of the system.

d. Academic Measures

i. Permanent Product Measure of Performance (PERMP).

The PERMP math test consists of a 10 minute validated math test containing 400 ability-appropriate math problems designed to measure a child's ability to stay on task and pay attention (Wigal et al., Journal of Attention Disorders, 10(2006): 92-111), which is related to their academic performance abilities. The participant is instructed to correctly answer as many problems as possible within 10 minutes, without skipping any problems. Each test is graded by counting the number of attempted and the number of correctly completed problems. The PERMP is a reliable and valid measure frequently used to evaluate response to stimulant medication (Wigal et al., Journal of Attention Disorders, 10(2006): 92-111). During the baseline session, participants took a math pretest PERMP to determine the appropriate math difficulty level. Participants were given tests based on the difficulty level established at pretest each time they took PERMP tests. During each administration of the academic measures, the PERMP was given 2 times: once before playing the game (Test 1) and once after playing the game (Test 2). During the baseline assessment for Group 2, in lieu of playing the game, there was a 30 minute break between Test 1 and Test 2. Different sets of problems were administered throughout the study to minimize practice effects.

ii. Woodcock-Johnson Third Edition (WJ-III).

The WJ-III Tests of Achievement includes a set of tests that assess achievement in reading and mathematics, written and oral language ability, and curricular knowledge (DuPaul et al., Journal of Abnormal Child Psychology, 34 (2006): 635-648). Subtests can be administered to individuals or groups, and are normed to age and grade level. Three subtests were used in the current study (Reading Fluency, Math Fluency, and Understanding Directions) because they were closely related to attention abilities. Form A of the WJ-III was administered at the baseline session and Form B of the WJ-III was administered at the completion of training for Group 1 or the end of the nonpharmacological intervention for Group 2 to avoid administering the same test twice. No additional forms were available to evaluate maintenance of effects or the effect of training for Group 2.

e. Training

i. Gameplay

The FFM training system included the game (Cogoland©) on a PC laptop and an EEG headband with three frontal sensors (Zeo Sleep Manager™, Zeo, Inc., Boston, Mass.). The training consisted of a calibration exercise, 24 game sessions, and 10 skill transfer module sessions. The sessions lasted up to 30 min and were supervised by the TC to ensure completion.

During the initial calibration, the software created a discriminate EEG model based on the participants performance on computerized exercises intended to provoke states of attention and inattention. The EEG recording during the calibration was used in a scoring algorithm that produced an index related to the participants state of attention in near real time.

During each game session, participants played a 3D computerized graphic cognitive training game called Cogoland©. As the participants played the game, their EEG waves were recorded simultaneously via the EEG sensors embedded in the headband. The EEG waves were used to quantify a participant's level of attention in real time, which ultimately controlled the speed of the character in the game. Game sessions typically lasted between 15 and 20 minutes.

The game consisted of three attention and inhibition skill development levels as the FFM challenged the participant to move the user avatar quickly around a track while ignoring auditory and visual distractors. The second and third levels added tasks where the participants were required to jump for the correct target fruit and not jump for non-targeted fruit. Participants were rewarded with points for their correct jumps and non-jumps while points were deducted for incorrect commissions and omissions.

Skill transfer modules were played by each participant to increase skill retention for transfer to real-life applications. This skills transfer exercise contained multiple-choice questions that were matched to the participant's academic grade level so the newly learned attention and impulse inhibition skills would be exercised to optimize retention.

ii. FFM Training Schedule

The first training session consisted of a 15 to 20 minute calibration exercise followed by one round of Level 1 gameplay. During the second training session, the participants were asked to complete PERMP assessments before and after the training session. Participants continued to train 3 to 4 times per week for 6 to 8 weeks. On the even-numbered sessions (fourth visit, sixth visit, etc.), the skills transfer module was administered to the participants. During the 12th and 24th sessions, a pregame and postgame PERMP was administered instead of the skills transfer module. As the FFM training progressed, game skills development was increased. Participants moved to the second skill level set during Session 5 and to the third skill level set during Session 14.

Results

To establish the effects of FFM training versus the control group, all measures were analyzed using a 2 (group)×2 (test)×3 (site) repeated-measures ANOVA. There was no effect of site except for the Quotient®, so site was dropped from the repeated-measures ANOVAs for all other measures. Missing data were handled per protocol, so participants with missing data were not included for that analysis. Participants who dropped out before the end of the initial FFM intervention period were not included in the analyses. Those who dropped out during follow-up were still included in this primary analysis but were not included in analyses of maintenance effects. To characterize the effect and sustainability of the FFM training intervention, the pooled training data from both groups were entered into a 2 (group)×3 (test: pre-study, post-training, follow-up for Group 1; post-wait, post-training, follow-up for Group 2) repeated-measures ANOVA intervention analysis and any significant interactions were analyzed using post hoc t tests.

a. Behavioral Measures

i. ADHD-RS.

The post-study clinician ADHD-RS evaluation was not available for one participant in Group 1, so that participant was excluded from the analysis. The combined score on the ADHD-RS showed significant effects of group, F(1, 37)=17.668, p<0.001, η²=0.323; test, F(1, 37)=25.689, p<0.001, η²=0.410; and a Group×Test interaction, F(1, 37)=28.428, p<0.001, η²=0.434. The subscores for inattention and hyperactivity/impulsivity reflected the same pattern (see Table 3). This indicates that the control group's symptoms were slightly more severe than the FFM training group at the beginning of the study; however, this difference was smaller than the improvement seen in the FFM training group. The effect of time and interaction of group by time are indicative of a reduction of 36% in ADHD symptoms for the immediate FFM training group (Group 1).

In the intervention analysis of the pooled FFM training data, there was no effect of group, F(1, 29)=1.865, p=0.183, η²=0.060, and no interaction, F(1, 29)=00431, p=0.516, η²=0.015, but there was an effect of test, F(1, 29)=66.151, p<0.001, η²=0.695, indicating that both groups achieved the same degree of improvement with FFM training. Post hoc analyses also indicated that there was a significant difference between before FFM training and after training, as well as before FFM training and follow-up (all ps<0.001). The improvements due to FFM training were also maintained through the 3-month follow-up, as indicated by a lack of significant differences between the two time points. This pattern also held for each of the two subscores (see Table 3).

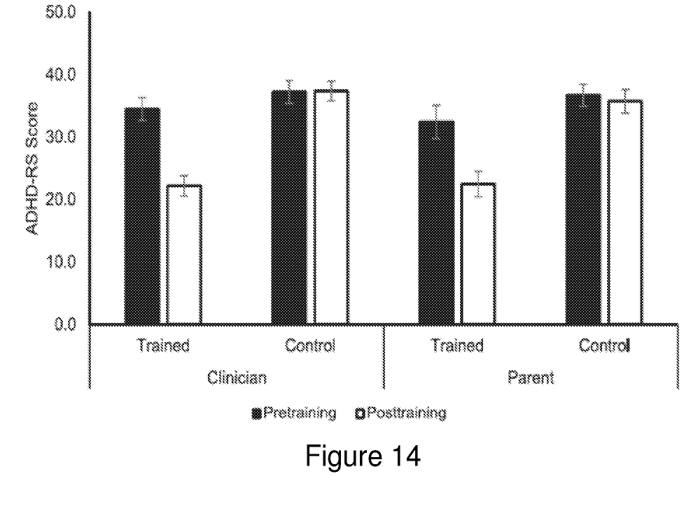

ADHD-RS scores reported by parents closely matched those reported by clinicians with effects of group, F(1, 38)=13.132, p<0.001, η²=257; time, F(1,38)˜14.695, p<0.001, η²=0.279; and a Group×Time interaction, F(1, 38)=6.237, p=0.017, η²=0.141. The control group was reported as being slightly more severe than the FFM training group before the study. Group 1's symptom severity improved by 31%, and Group 2 did not show any improvements. This improvement was also observed in the inattention and hyperactivity/impulsivity subs cores (Table 3). When looking at the effect of FFM training for both groups, the amount of improvement was the same for each group, as demonstrated by an effect of time, but no effect of group or interaction (ps>0.2) in the pooled intervention analysis. As was seen for the clinician ratings, the FFM improvements were still evident at the 3-month follow-up for all scores (see FIG. 14). The reductions in symptoms reported on the ADHD-RS indicate that FFM training led to improvements that would be categorized as moving from moderately severe symptoms to near normal levels.

b. CGI

At the second clinician consultation, the severity measure of the CGI was not completed for seven participants (only improvement was noted), so they could not be included in this analysis. The ANOVA comparing training with standard non-pharmacological care reported effects of both group, F(1, 31)=7.110, p=0.012, η²=0.187, and time, F(1, 31)=13.627, p=0.0009, η²=0.305, and a significant interaction of the two, F(1, 31)=12.201, p=0.001, η²=0.282 (see Table 3). The effect of group was related to the fact that the FFM training group was rated as being slightly less severe than the control group, as was seen on the ADHD-RS scores. However, this initial difference was much less than the amount of improvement seen from the FFM training group and equal to the change the control group experienced. An analysis of the pooled effect of FFM training for each group confirmed that the slight difference in severity did not change the effectiveness of the FFM training-effect of group and interaction with time ps>0.9; effect of time: F(1, 26)=37.471, p<0.0005, η²=0.590. The CGI indicates that FFM training led to improvements from being categorized as moderately ill to only being mildly ill.

TABLE 3
Mean Scores (Standard Deviation) Before and After Training for Both Groups.

| | Pre-training | | | | Post-training | | | |
|---|---|---|---|---|---|---|---|---|
| Measure | Trained | | Control | | Trained | | Control | |
| ADHD-RS | | | | | | | | |
| Clinician | 34.5 | (8.0) | 37.2 | (8.2) | 22.2 | (9.7) | 37.4 | (9.7)*** |
| Parent | 32.4 | (11.9) | 36.7 | (8.2) | 22.5 | (14.6) | 35.7 | (9.3)*** |
| CGI | 4.32 | (0.77) | 4.42 | (0.69) | 3.14 | (1.17) | 4.32 | (0.58)*** |
| Quotient | | | | | | | | |
| Inattention score | 6.79 | (2.10) | 6.64 | (2.46) | 7.93 | (2.03) | 6.65 | (2.54) |
| PERMP | | | | | | | | |
| Test 1 correct | 76.3 | (41.8) | 77.4 | (54.7) | 103.3 | (56.3) | 76.5 | (11.7)* |
| Test 1 attempted | 78.4 | (42.2) | 83.5 | (55.3) | 105.3 | (56.9) | 71.9 | (56.7)* |
| Test 2 correct | 68.6 | (39.2) | 59.9 | (52.8) | 101.4 | (61.3) | 68.9 | (51.3)* |
| Test 2 attempted | 69.9 | (39.2) | 65.5 | (53.1) | 103.1 | (61.9) | 73.5 | (50.3)* |
| WJ-III | | | | | | | | |
| Reading fluency | | | | | | | | |
| Age equivalent | 10.94 | (2.77) | 12.77 | (3.35) | 11.14 | (3.19) | 12.89 | (3.52) |
| Grade equivalent | 5.57 | (2.79) | 7.29 | (3.18) | 5.74 | (3.15) | 7.38 | (3.25) |
| Math fluency | | | | | | | | |
| Age equivalent | 9.68 | (2.41) | 9.71 | (2.83) | 10.04 | (2.79) | 10.58 | (4.34) |
| Grade equivalent | 4.27 | (2.38) | 4.41 | (0.74) | 4.50 | (2.81) | 4.87 | (3.27) |
| Understanding directions | | | | | | | | |
| Age equivalent | 10.27 | (2.97) | 10.76 | (3.02) | 11.62 | (3.96) | 10.88 | (3.01) |
| Grade equivalent | 5.04 | (3.56) | 5.64 | (3.59) | 6.64 | (4.65) | 5.81 | (3.57) |

*p < .05.
***p < .001.

c. Quotient® ADHD System.

There were no significant main effects or interactions on the global measure of the Quotient® ADHD System (all ps>0.2). For the inattention subscore, there was an effect of time, F(1, 18)=5.207, p=0.035, η²=0.224, and a trend of a Group×Time interaction, F(1, 18)=3.511, p=0.077, η²=0.163, for the inattention score. Unlike the other measures, this was due to the FFM training group scores worsening, while the control group remained the same. On the motion measure, there was a significant effect of site, F(1, 18)=6.364, p=0.0213, η²=0.261, where one site had a consistently higher score for motion, a trend that can also be seen in the ADHD-RS hyperactivity scores, although it was not significant in that measure.

Looking at the pooled effect of FFM training for each group, the same pattern emerges in the inattention score. There was a significant effect of time for both the inattention, F(1, 18)=10.718, p=0.004, η²=0.373, and global, F(1, 18)=2.353, p=0.030, η²=0.236, scores. There were no significant interactions (ps>0.3), but as was seen in other measures, the FFM trained group was less severe in the motion score than the control group, F(1, 18)=5.509, p=0.031, η²=0.234. The effects of time were due to worsening inattention scores after FFM training (see Table 3).

b. Academic Measures

a. PERMP

The performance on the PERMP is listed in Table 3. For all four measures—correct and attempted both before (Test 1) and after (Test 2) game play—the effect of time was significant (all ps<0.02, η²s>0.150) and there was a significant interaction between group and time (ps<0.01, η²s>0.150), but no significant effect of group (ps>0.4). Accuracy was generally high on this test. The majority of problems attempted were correct (>90%). The lack of effect for group indicates that both groups were able to complete the same number of problems at the beginning of the study.

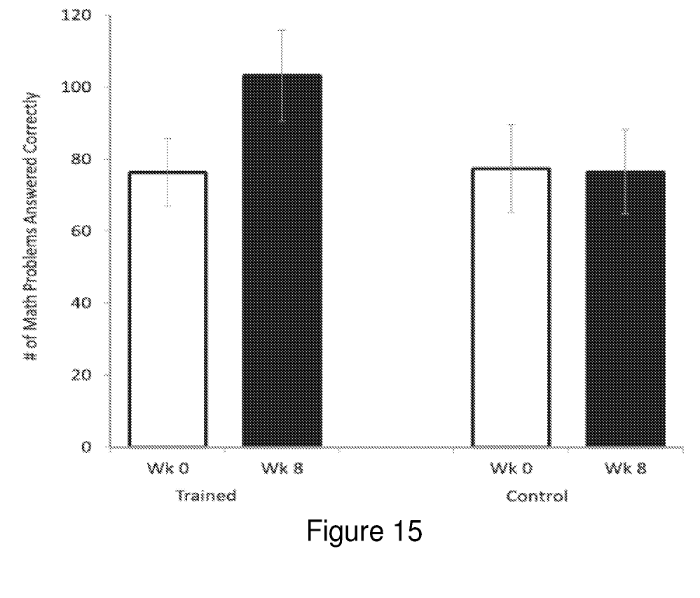

After the initial 8 weeks, Group 1 increased the number of problems they could complete in the time limit by 26% on average, whereas Group 2 did not show any increase in the number of problems (see FIG. 15). There was no difference in the gains between groups once FFM training was completed (ps>0.5) and no interactions (ps>0.1). There was the effect of time (ps<0.004, η²s>0.200) showing improvement with FFM training for both groups. However, Bonferroni-adjusted post hoc t tests indicated that the statistical significance of the increase in performance was not sustained at 3 months.

b. WJ-III.

There were only two versions of WJ-III available (Forms A and B), so the WJ-III assessment was only completed at baseline and after the first 8 weeks (FFM training for Group 1; standard non-pharmacological care for Group 2). Six participants did not complete the WJ-III because a different test was initially used and found not to be appropriate to this cohort. In addition, one participant who completed Reading and Math Fluency tests did not complete Understanding Directions. All subtests reported both age and grade equivalents, and repeated-measures ANOVAs were completed for both. For Math Fluency, there was an effect of time in age and grade level, age: F(1, 29)=7.037, p=0.013, η²=0.195; grade: F(1, 29)=14.076, p<0.001, η²=0.327; however, the age effect disappeared when adjusted for the 2 months that had passed between tests. There were no effects of group or interactions (ps>0.2). Reading Fluency did not show any improvements (ps>0.1). In Understanding Directions, there was not an effect of group (ps>0.9). There was a trend of improvement over time and an interaction between time and group, but these were not significant and had very small effect sizes (ps>0.05, η²<0.1; see Table 3).

### DISCUSSION

After 8 weeks of either FFM training or nonpharmacological community care options, the FFM training group showed improvements in ADHD symptoms whereas the control group did not demonstrate meaningful improvement. Clinicians reported a 36% reduction in symptoms on the ADHD-RS, with similar improvement reported on the CGI. Parents also reported reduction in the symptoms after FFM training of approximately 31%. The nonpharmacological interventions did not lead to significant improvement of symptoms. The FFM training group also showed some improvement on measures of academic performance, demonstrating a greater ability to stay on task and thereby correctly answering more questions on the PERMP after training. A trend was also observed toward improvement in the FFM training group's ability to control their impulses and follow directions on the WJ-III Understanding Directions test. The two groups did not differ on the measures of Reading Fluency and Math Fluency, which may have been due to the short time limit of these tests. Although Math Fluency and the PERMP are similar tests, improvements were observed the 10-minute time limit of the PERMP, but not on the Math Fluency test with the 3-minute duration. All FFM training improvements were also sustained 3 months after training ended. While in some cases applying Bonferroni corrections for multiple comparisons led to improvements losing statistical significance, numerically they did not return to baseline levels.

All of the academic measures were carefully chosen to minimize retesting (practice) effects by having multiple versions of the test. The WJ-III is designed and has been validated to be used across multiple time periods using different forms of the test (Forms A and B). The PERMP is designed to be administered multiple times within 1 day and has been validated as a measure that is sensitive to medication levels throughout the day, confirming a lack of practice effects on different versions of the test. In light of this, improvements merely due to having experience taking the test would not be expected. Even with this effort, the data overall show slightly higher scores after retest. However, these retest improvements were very small and not statistically significant.

FFM improvements from training on the Quotient® ADHD System were not observed. Although this finding was unexpected, a further review of the literature indicates a lack of correspondence between continuous performance tasks like the Quotient® and symptomatology in ADHD (Jonsdottir et al., Archives of Clinical Neuropsychology 21(2006) 383-394). This may be due to ADHD inattention being related to a general construct of “attention” and not any one particular type of attention ability (Castellanos et al., Biological Psychiatry 63(2006) 332-337; Jonsdottir et al., Archives of Clinical Neuropsychology 21 (2006) 383-394. Other treatments for ADHD symptoms have reported a similar lack of improvement on these computer-based performance tasks. In addition to a lack of improvement overall, the FFM training group displayed a worsening of symptoms, which may be driven by two outliers in the group.

This randomized, controlled trial demonstrates that this FFM system is a superior option to current nonpharmacological interventions provided in community clinics to treat children with ADHD. Many of the participants in this study had not previously been on medication, and their parents were seeking non-medication treatment options. Although carefully controlled behavior therapies can be effective in alleviating ADHD symptoms, the treatment options currently available in a more general community care setting, such as the nonpharmacological approaches used in the control group, lead to limited reductions in severity of ADHD symptomatology, especially after only 8 weeks. In contrast, this FFM training led to significant and sustained reductions in ADHD symptomatology and in selected measures of academic performance. The nonpharmacological approaches generally show better performance when they are extended over longer periods and combined with medication treatment. Even without medication, this FFM training system led to significant and sustained severity reductions, so FFM training may be a viable first-line treatment option for ADHD. The potential for FFM training to enhance the effect of medication or reduce the required maintenance dose of medication is an important question for future studies.

In conclusion, an FFM training system in a randomized, controlled study of 8- to 12-year-old children appeared to be an effective intervention for the treatment of ADHD and improving academic performance. The FFM system led to more significant and sustained (a) reductions in the severity of ADHD symptomatology and (b) improvements in academic performance abilities than the standard nonpharmacological intervention options used by the control group. This FFM training represents a potential new non-pharmaceutical intervention for the treatment of ADHD. The FFM training was also shown to improve objective measures of academic performance, demonstrating that what was learned in the FFM training effectively transferred to near real-world behavior (i.e., improved behavior at home) and to academic abilities that are far removed from the training itself.

### Example 13. Mission Performance Reports

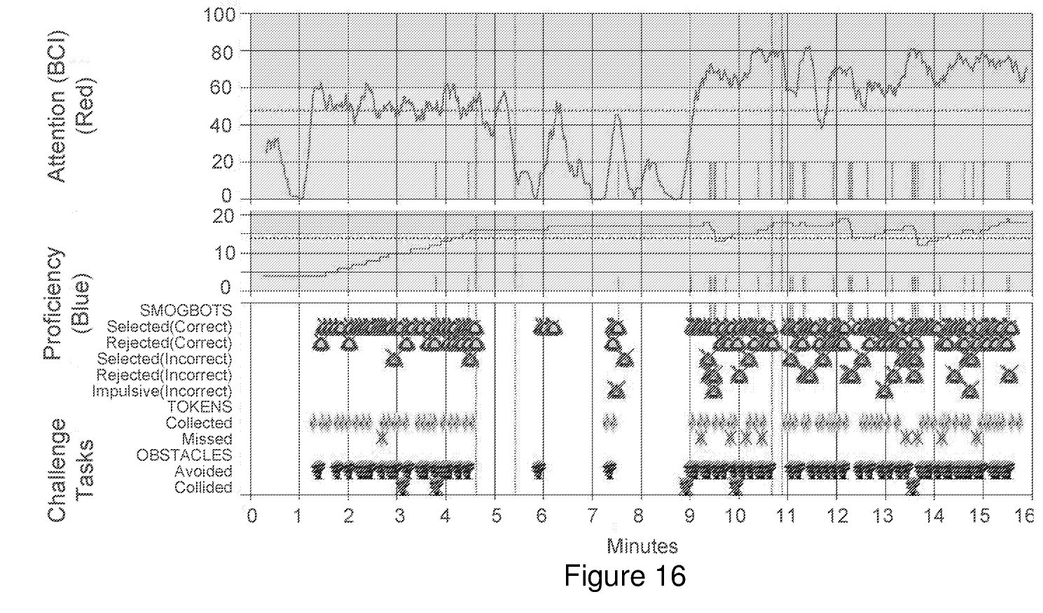

The methods and systems of the invention can include generating a mission performance report (MPR) for a training session completed by a user. For example, an MPR can depict, over time, an attention state level and successful or unsuccessful attempts at achieving challenge tasks. These are reported in parallel allowing for review of attention state levels before, during, and after each challenge task. An exemplary MPR for a user completing a mission of an exemplary game is provided in FIG. 16. The MPR can include (i) a global attention score and/or a global composite score calculated from the user's cognitive skill performance over the course of the training session; (ii) an attention state level or series of attention state levels calculated from the EEG brain activity signals collected from the user during the training session, (iii) the individual results of challenge tasks, and/or (iv) the game difficulty level (proficiency) achieved by the user during the training session. Individual scores are determined for each cognitive skill. Each score is based on a count of either an individual or aggregates of attention (e.g., attention state levels) over a defined interval or correct/incorrect response(s) for a unique set of conditions (e.g., one or more challenge tasks). Individual score calculations include those exemplified below.

Focused Attention

Focused attention is measured using a percentage of attention state levels over a predetermined threshold level for a session. For example, a predetermined threshold can be set at 60%. In this case, a count of all instances of an attention state level having a value over 60 is used to determine a raw focused attention value. This raw focused attention state value, divided by the total number of attention state levels for a session, is the percentage of attention state levels over 60% and becomes a focused attention score when scaled from 0 to 100.

Sustained Attention

Sustained attention is measured using a percentage of adjacent attention state levels (e.g., attention state levels that are acquired at sequential time points), within a given session, having differences within a predetermined threshold variance. A count of all instances in which the change in attention state level between adjacent attention state measurements is less than the predetermined threshold variance is used to determine sustained attention. For example, a predetermined threshold variance can be set to 10%. In this case, a sustained attention state value corresponds to each sequential attention state level having a value within 10% relative to its preceding attention state level. Additional criteria may be introduced, such as a requirement for a sustained attention state level value to correspond to an attention state level having a predetermined threshold level (e.g., a value that is the same or different from the predetermined threshold level used in calculating the focused attention score). The count of sustained attention state levels divided by the total number of attention state levels for the session becomes a sustained attention score when scaled from 0 to 100%.

Selective Attention

Selective attention is measured using a percentage of correct responses to challenge tasks for a session. (See Example 3 for exemplary challenge tasks.) All instances of correct responses to challenge tasks (e.g., correctly selected or correctly rejected targets) where none or one of the elements in a group of elements is a valid target are counted. The count of raw selective attention divided by the total number of targets or groups of targets (e.g., clusters of targets, e.g., opportunities) for the session becomes the selective attention score from when scaled from 0 to 100%.

Alternating Attention

Alternating attention is measured using a percentage of correct responses measured for unique alternating challenge tasks for a session. Alternating challenge tasks can include target rule switches, or alternating target rules (e.g., rules identifying targets as objects having a specific set of characteristics, e.g., shapes, colors, and symbols). Target rules refer to the use of consistent types like shape, color, and symbol with differing values. In challenge tasks testing alternating attention, a correct selection may require the user to consider all of the characteristics of an object (e.g., all three of shape, color, and symbol) when determining whether or not the object is a valid target. All instances of correct responses (correctly selected or correctly rejected) to challenge tasks directly after a switch in target rules (e.g., the first challenge task following the change in rules) are counted. The count of raw alternating attention divided by the total number of switches in target rules for the session becomes the alternating attention score from when scaled from 0 to 100%.

Divided Attention

Measuring divided attention is distinct from measuring alternating attention in that a user is prompted to respond to groups of objects having one or more matching attributes rather than having all attributes match one another. Divided attention is measured using a percentage of correct responses measured for unique challenge tasks that involve a target type switch. Target type refers to the different sets of types, such as shape, color, and symbol, or lack of each. A target type switch can be a switch from one set of target types (i.e. color and shape) to a different set (i.e. symbol). Instead of looking for all three attributes at the same time, the player must look for one or more matching types and ignore others in determining a valid target. All instances of correct responses (correctly selected or correctly rejected) to challenge tasks directly after a target type switch are counted. The count of raw alternating attention divided by the total number of target type switches for the session becomes the divided attention score from when scaled from 0 to 100%.

Cognitive Inhibition

Cognitive inhibition is measured using a percentage of attention state levels over a predetermined threshold level for a specific portion of a session. For example, a predetermined threshold level can be set at 60%. In this case, a count of all instances of an attention state level having a value of at least 60% for a period in which the player may be more susceptible to day dreaming, e.g., during the first 60 seconds of a mission, is used to determine a raw cognitive inhibition value. The count of raw cognitive inhibition values divided by the total number of attention state levels for the specified portion of the session becomes the cognitive inhibition score from when scaled from 0 to 100%.

Behavioral Inhibition

Behavioral inhibition is measured using a percentage of correct responses to challenge tasks requiring rejection of a target for a session. All instances of correct responses (correctly selected or correctly rejected) to challenge tasks requiring a rejection of a target are counted as a raw behavioral inhibition value. The raw behavioral inhibition value divided by the total number of targets to be rejected for the session becomes the behavioral inhibition score when scaled from 0 to 100%.

Novelty Inhibition

Novelty inhibition is measured using a percentage of correct responses for challenge tasks occurring while experiencing irrelevant stimuli. The raw novelty inhibition value divided by the total number of targets or groups of targets (e.g., challenge tasks) for the session becomes the novelty inhibition score when scaled from 0 to 100%.

Motivational Inhibition

Motivational inhibition is measured using a percentage of correct responses measured directly after an incorrect response (e.g., a challenge task immediately following an incorrect selection or an incorrect rejection). All instances of correct responses (correctly selected or correctly rejected) to challenge tasks where the previous response was incorrect are counted as the raw motivational inhibition value. The raw motivational inhibition value divided by the total number of incorrect responses for the session becomes the motivational inhibition score when scaled from 0 to 100%.

Interference Control

Interference control is measured using a percentage of incorrect responses to challenge tasks for a session. All instances of incorrect responses to challenge tasks where none or one of the elements in a group of elements is a valid target are counted as the raw interference control value. The raw interference control value divided by the total number of challenge tasks for the session becomes the interference control score when scaled from 0 to 100% and inverted. Inversion of the final value is performed to allow interference control to the tracked as an increasing value as the user improves, to conform to the remaining scores. This allows the value to be averaged with other scores to generate, e.g., a global composite score.

Inner Voice

Inner voice is measured using a count of positive changes in attention state level measured after falling below a predetermined threshold. All instances of significant positive changes in attention level exceeding a predetermined threshold level after the attention state level had fallen below a predetermined minimum threshold attention state level are counted. The inner voice value becomes the inner voice metric when scaled from 0 to 100% and inverted. Inversion of the final value is performed to allow inner voice to be tracked as an increasing value as the user improves, to confirm to the remaining scores. This allows the value to be averaged with the other scores to generate a global composite score.

Delay of Gratification

Delay of gratification is measured using a percentage of correct responses to challenge tasks during a secondary engagement for a session. All instances of correct responses to primary challenge tasks where a secondary (e.g., collision/avoidance) challenge task is presented simultaneously or within a predetermined timeframe are counted as the raw delay of gratification value. The raw delay of gratification value divided by the number of total instances of simultaneous or near-simultaneous primary and secondary engagement for the session becomes the delay of gratification score when scaled from 0 to 100.

Self-Regulation

Self-regulation is measured using a percentage of correct responses to challenge tasks during multiple secondary (e.g., collision/avoidance) challenge tasks for a session. All instances of correct responses to primary challenge tasks in during which a secondary collision/avoidance and collection challenge task occurs simultaneously are counted as the raw self-gratification value. The raw self-gratification value divided by the number of total instances of simultaneous primary and multiple secondary engagements (e.g., challenge tasks) for the session becomes the self-regulation score when scaled from 0 to 100.

The formula for each of the 13 scores is shown in Table 4, below.

TABLE 4
Exemplary Formulae for Attention Scores

| | | Improvement |
|---|---|---|
| Skill | Formula for Score Calculation | Trend |
| Focused Attention | % of attention state levels ≥60 | Higher % |
| Sustained Attention | % of attention state levels ≥40, where comparison to | Higher % |
| | previous attention state level has a difference of ≤10 | |
| Cognitive Inhibition | % of attention state levels ≥60 over first 60 seconds of a | Higher % |
| | mission when the primary activity is paying attention | |
| Behavioral Inhibition | CR/(CR + IS) | Higher % |
| Selective Attention | (CS + CR)/(Total Opportunities) | Higher % |
| | (CS + CR) may be calculated by clusters of stimuli | |
| | A cluster of stimuli may present one opportunity | |
| Alternating Attention | (CS + CR directly after target rules switch)/(Total number of | Higher % |
| | target rules switches) | |
| | A target rules switch can be a change of selection | |
| | criteria (e.g., a correct response is selecting an object | |
| | based on shape rather than symbol) | |
| Divided Attention | (CS + CR)/(Total Opportunities) | Higher % |
| Interference Control | (IS)/(Total Opportunities) | Lower % |
| Novelty Inhibition | (CS + CR)/(Total Opportunities) | Higher % |
| | (CS + CR) may be calculated by clusters of stimuli | |
| | A cluster of stimuli may present one opportunity | |
| Delay of Gratification | (CS)/(Total Opportunities) | Higher % |
| Inner Voice | The number of instances where an attention state level | Lower counts |
| | increased by ≥30 when the previous attention state level | |
| | was <40 (40 is an exemplary inner voice threshold) | |
| Motivational Inhibition | ((CS + CR) after IS or IR)/(CS + CR + IS + IR) | Higher % |
| | This measures % correct actions directly after an | |
| | incorrect action. | |
| Self-Regulation | (CS)/(Total Opportunities) | Higher % |

CS stands for “correctly selected,” CR stands for “correctly rejected,” IS stands for “incorrectly selected,” and IR stands for “incorrectly rejected.”

### Example 14. Summary Progress Reports

Once individual scores are generated for a session they are averaged based on the overall cognitive skill (attention-associated skill or impulse/inhibition-associated skill) and weighted according to the game level. The resulting value is taken as a global attention score or a global composite (e.g., a composite of attention and impulse inhibition) score, and plotted by the day (from start) on which training was performed in separate charts for focused/sustained attention and a composite of attention and impulse/inhibition control. The resulting graph is included as a Summary Progress Report. Calculated lines based on least squares” methodology (e.g., linear or nonlinear, e.g., curvilinear least squares methodology) are calculated for the data in each chart. Lines based on the results of the calculations are added to their corresponding charts.

The results of the least squares calculation are used to calculate the “pre” and “post” values for the (focused/sustained or impulse/inhibition) scores and the “% change” observed between the start and end of training.

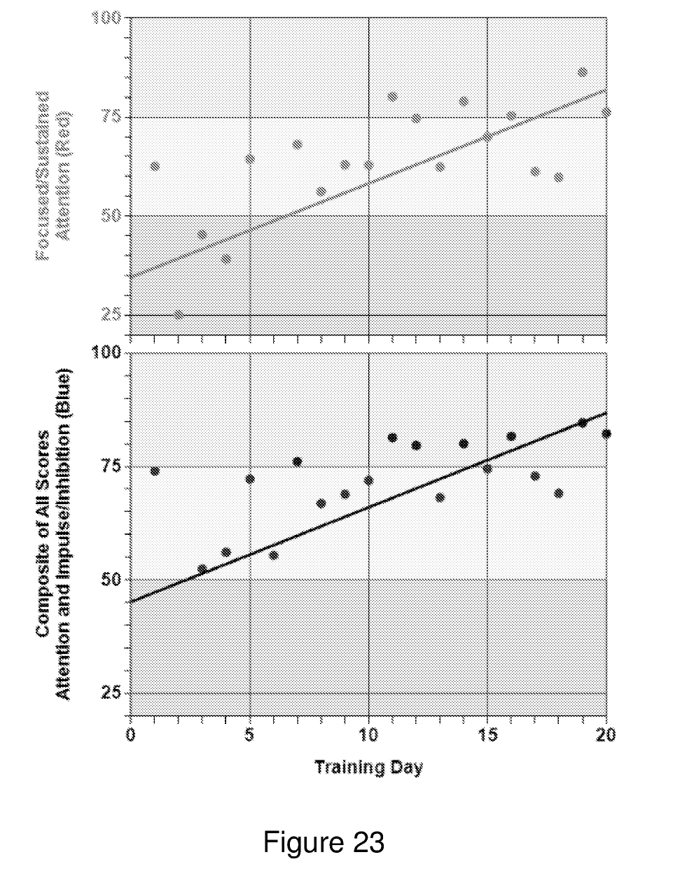

Thus, the methods and systems of the invention can include generating a summary progress report for a user following a predetermined number of training sessions over time (e.g., 3 to 8 weeks). The summary progress report can include a depiction of (i) the change in the user's global attention score for a period of training undertaken by the user over a period of days, and (ii) the change in the user's composite score for training sessions undertaken by the user over a period of days. An exemplary summary progress report is provided in FIG. 23.

### Example 15. Example User Cognitive Skills Performance History

The following description is set forth to as an example to illustrate how the cognitive skills underlying the executive functions of attention and impulse inhibition can be taught, tested for retention and transfer to real life, and monitored for regimen adherence within the context of the adventure story line of a game based learning curriculum and its series of learning missions, according to one embodiment of the invention.

The game's story line is established using animated videos (e.g., cut-scenes), presented between gameplay segments, and is reinforced during gameplay using voice-over dialog between characters and in the visual design of the environment (i.e. the scenery). The story line centers on the journey of the user avatar, Skylar, who can be male or female depending on the user's selection. The female form of Skylar is used for the story summary below.

The story line is a key instructional element of the virtual learning curriculum. The user avatar is a stand-in for the user (e.g., a child who must learn to control (e.g., optimize) his or her attention and impulsivity control (e.g., impulse-inhibition-associated cognitive skills) in order to be successful in each mission of the adventure game based story line). The peer and mentor characters that the user avatar encounters direct their dialog to the avatar, but the true target is the user. The peer and mentor characters provide positive reinforcement and environment for the user's learning, practicing and demonstration of these cognitive skills, encouraging of the user's persistence toward mastering game challenge tasks, and suggestive of practical strategies for maintaining focus and meeting the emerging challenge tasks. Through the adventure story, the user avatar demonstrates that any user can improve the underlying cognitive skills of attention and impulse control, become an exemplar and leader to others, and reach their personal goals.

Story Summary

Skylar is an ordinary middle-schooler just trying to get through his or her day at school when he or she finds themselves magically transported to Lightbreaker Novo—a space transport on its way to respond to an emergency beacon on the remote planet of Geoshale. The Novo is commanded by the GSTAR Agents, an intergalactic search and rescue team. A GSTAR Commander, Agent Wyll, pulls Skylar into the mission before he or she can object. Assisting the Agents is Sentient Belen, a member of an elite order dedicated to mastering the capabilities of the mind. Wyll and Belen have Skylar complete the “Calabrus,” a calibration activity that allows Skylar to utilize an AV™ Headset, a fictionalized version of the same EEG AV™ headset worn by the user.

Upon embarking on the planet surface, most of the Agents are put out of commission by a condition that makes them disoriented and unresponsive. The culprit: the thick fog covering the planet. Skylar and Wyll escape the effects of the fog when Wyll dons his AV™ Headset and blasts it away with the power of his attentive mind. While Wyll rounds up the affected Agents, Skylar is entrusted with the task of running to the only landmark visible above the fog: a tower constructed by the planet's native inhabitants. What he or she finds at the tower will be his or her first clue to help unravel the planet's many mysteries: What happened to the civilization that called them to the planet? What is the nature of the fog? How can they reverse its effects? And, most importantly to Skylar, how will he or she get back home?

Each of the 15 missions in the game advances the story toward its resolution while advancing both Skylar/the user toward greater acquisition of the cognitive skills underlying attention and impulse control. Table 5, below, outlines, for each mission, the primary story objectives and Table 6, below, provides the targeted cognitive skills taught and measured in each of 15 missions.

TABLE 5
Mission Sequence

| Level Goal | Mission | Runner Objective | Transference | User Learns |
|---|---|---|---|---|
| 1. Find out what | Mission 1 | Reach the tower to find | Psychomotor | Attention |
| happened to the | | the map of Geoshale | Vigilance Task - | Maintenance: |
| Shaliens | | | Unlock the Map | I can manage |
| | Mission 2 | Reach the Power | Analyze samples | distractions to |
| | | Generator to restore | of the fog | control my attention |
| | | power to the planet | | state level. |
| | Mission 3 | Reach the Beacon to | | |
| | | play the emergency | | |
| | | message | | |
| 2. Make first | Mission 4 | Reach Com Relay #1 | Analyze complex | Behavioral |
| contact with the | Mission 5 | Reach Com Relay #2 | fog samples | Inhibition: |
| Shalien refugees | Mission 6 | Reach Com Relay #3 to | harvested from | I can keep my cool, |
| | | contact the Shalians | within the | control my actions, |
| | | | Smogbots | and resist negative |
| | | | | impulses. |
| 3. Collect the | Mission 7 | Reach item #1. | Test how the fog | Selective Attention: |
| powerful | Mission 8 | Reach item #2. | reacts to the | I can focus on the |
| substances to | Mission 9 | Reach item #3 | powerful | right thing; find the |
| make an antidote | | | substances | signal in the noise. |
| 4. Liberate the | Mission 10 | Destroy the sentinel | Transform fog | Alternating |
| Shalien city and | | Smogbot to reach the | samples into an | Attention: |
| deliver the | | city. | antidote | I can be flexible and |
| antidote | Mission 11 | Deliver antidote #1 to | | switch gears quickly. |
| | | the Shaliens | | |
| | Mission 12 | Deliver antidote #2 to | | |
| | | the Shaliens | | |
| 5. Teach others | Mission 13 | Search for Wyll within a | Test the blue fog | Novelty Inhibition |
| how to manage | | forbidden zone | | and Mastery: |
| the fog. Confront | Mission 14 | Reach Wyll's campsite | | I can ignore new |
| the source of the | | to rescue him | | distractions and |
| fog. | Mission 15 | Reach the fog epicenter | Mix new antidote | follow through to |
| | | to confront the Master | for Wyll | completing my |
| | | Smogbot | | goals. |

TABLE 6
Cognitive Skills Taught and Measured in each Mission

| Level Goal | Mission | Cognitive Skills Measured |
|---|---|---|
| 1. Find out what | Mission 1 | Focused Attention, Sustained Attention, Cognitive |
| happened to the | Mission 2 | Inhibition, Inner Voice |
| Shaliens | Mission 3 | |
| 2. Make first contact | Mission 4 | Focused Attention, Sustained Attention, Cognitive |
| with the Shalien | Mission 5 | Inhibition, Inner Voice, Behavioral Inhibition, Delay |
| refugees | Mission 6 | of Gratification, Motivational Inhibition, Self- |
| | | regulation |
| 3. Collect the powerful | Mission 7 | Focused Attention, Sustained Attention, Cognitive |
| substances to make an | Mission 8 | Inhibition, Inner Voice, Interference Control, Delay |
| antidote | Mission 9 | of Gratification, Motivational Inhibition, Self- |
| | | regulation, Selective Attention |
| 4. Liberate the Shalien | Mission 10 | Focused Attention, Sustained Attention, Cognitive |
| city and deliver the | Mission 11 | Inhibition, Inner Voice, Delay of Gratification, |
| antidote | Mission 12 | Motivational Inhibition, Self-regulation, |
| 5. Teach others how to | Mission 13 | Focused Attention, Sustained Attention, Cognitive |
| manage the fog. | | Inhibition, Behavioral Inhibition, Novelty Inhibition, |
| Confront the source of | | Delay of Gratification, Inner Voice, Motivational |
| the fog. | | Inhibition, Self-regulation |
| | Mission 14 | Focused Attention, Sustained Attention, Cognitive |
| | | Inhibition, Selective Attention, Interference Control, |
| | | Novelty Inhibition, Delay of Gratification, Inner |
| | | Voice, Motivational Inhibition, Self-regulation |
| | Mission 15 | Focused Attention, Sustained Attention, Cognitive |
| | | Inhibition, Alternating Attention, Divided Attention, |
| | | Novelty Inhibition, Delay of Gratification, Inner |
| | | Voice, Motivational Inhibition, Self-regulation |

Mission Summary

The gameplay activities that the user must perform evolve throughout the game. With each mission, game mechanics representing new ways of training or measuring the cognitive skills are added, and/or the user must demonstrate higher performance levels at existing challenge tasks in order to progress. For example, sustained attention, which is taught in Mission 1, must be maintained throughout each and every mission to propel the user avatar through each adventure. As the missions progress, the number of skills being taught, utilized, monitored, and measured accumulates. For reporting purposes a global attention score and/or a global composite score are derived for each mission or training session. The global attention score is a composite score (e.g., an average or weighted average) of any and all attention scores (e.g., focused attention, sustained attention, selective attention, alternating attention, and divided attention) while the global composite score is a composite (e.g., an average or weighted average) of all attention and impulse/inhibition scores (e.g., focused attention, sustained attention, cognitive inhibition, behavioral inhibition, selective attention, alternating attention, divided attention, interference control, novelty inhibition, delay of gratification, inner voice, motivational inhibition, or self-regulation) A summary of each mission is provided below.

Mission 1:

Mission 1 begins with a set of calibrations. First, a Biocal calibration is performed, wherein the user must perform six facial muscle movements when prompted to partially personalize the EEG signal-processing algorithm. Next, a psychomotor vigilance task (PVT) calibration is performed, wherein the user must monitor the screen and respond quickly to a simple stimuli for 10 minutes in order to complete the EEG signal-processing personalization for determination of a personalized attention state algorithm for each user.

Next, training mechanics are introduced to the user. First, the user is introduced to attention-driven running. The user's attention state level, expressed as a value between 0 and 100%, determines the speed of the avatar as it runs forward along a 3D path. Next, environmental distractors are introduced. Animated, noisy objects appear adjacent to the avatar's path to distract the user from their goal. After a period of time, shiny crystals appear along the path that the user is encouraged to collect.

A peer character (Wyll) for cognitive skill guidance is then introduced. First, an Attention Mantra is provided to the user. The peer character provides the user with instructions for improving their focused attention state levels, and a memorable phrase that can be repeated in order to internalize those instructions, “Clear minds. Focus forward.” The peer character assures the user that difficulty controlling the speed of the avatar at the beginning of training is totally normal, and encourages the user to keep trying. When the measurement of the user's attention state level drops below a set threshold, the peer character provides additional encouragements and instructional reminders.

Training performance goals are then pursued by the user. Sustained attention is tested, wherein the user must sustain their attention state level above a set threshold to fill a “power meter.” The challenge level is very low. The user must fill the power meter to a relatively low mark in order to succeed.

A score on a scale of 1-100% is calculated for each cognitive skill measured in Mission 1, according to the formulae set forth in Example 13. Specifically, Mission 1 includes calculation of a focused attention score, sustained attention score, cognitive inhibition score, and inner voice score.

At the end of Mission 1, a mission progress report is generated to show the details of the user's performance. A global attention score is calculated based on the user's performance with respect to attention scores. Additionally, a global composite score is calculated based on the user's performance with respect to attention and impulse/inhibition scores. The Mission 1 global attention score is indicative of the user's ability to focus and sustain his or her attention. The completion of Mission 1 may mark the completion of the user's first training session, in which case the Mission 1 global attention score becomes the first data point on the Focused/Sustained Attention graph of the user's summary performance report (see, for example, FIG. 23, top chart), and the Mission 1 global composite score becomes the first data point on the user's Composite of All Scores graph of the summary performance report (see, for example, FIG. 23, bottom chart).

Mission 2

Mission 2 begins by introducing the training mechanics to the user. The user controls the avatar via touch inputs to avoid physical obstacles that appear in the path. Colliding with an obstacle slows the user avatar through the adventure story.

Next, the peer character guidance is introduced to the user. The peer character continues to encourage the user to keep trying the targeted cognitive skill(s) even if the user cannot get the avatar to move fast due to low attention state levels. The peer character explains how the Power Meter, which measures the player's sustained attention performance in Missions 1-3, is critical to progress in the game.

Cognitive skill training performance goals are then modeled in front of the user. Sustained attention is trained and measured in Mission 2, with a challenge level of medium.

Next, mechanics of the skill transfer module are introduced to the user avatar. The user must monitor the screen for the appearance of specific molecules and quickly tap molecules when they appear. Additionally, the user is prompted to maintain their attention state levels above a set threshold in order to analyze or “decode” the selected molecules. The transfer module monitors sustained attention state levels by measuring the number of molecules decoded by the user. The challenge level of this transfer module is low. The user must select and decode a relatively small number of molecules to succeed.

A score on a scale of 1-100% is calculated for each cognitive skill measured in Mission 2, according to the formulae set forth in Example 13. Specifically, Mission 2 includes calculation of a focused attention score, sustained attention score, cognitive inhibition score, and inner voice score.

At the end of Mission 2, a mission progress report is generated to show the details of the user's performance. A global attention score is calculated based on the user's performance with respect to attention scores. Additionally, a global composite score is calculated based on the user's performance with respect to attention and impulse/inhibition scores. The Mission 2 global attention score is indicative of the user's ability to focus and sustain his or her attention. The completion of Mission 2 may mark the completion of the user's second training session, in which case the Mission 2 global attention score becomes the second data point on the user's Focused/Sustained Attention graph of the summary performance report (see, for example, FIG. 23, top chart), and the Mission 2 global composite score becomes the second data point on the user's Composite of All Scores graph of the summary performance report (see, for example, FIG. 23, bottom chart).

Mission 3

Mission 3 builds from the cognitive skills training and retention modules of the first two missions. The goal is to teach the user how to sustain high levels of attention states. In Mission 3, the challenge level is high. The transfer module positions the user to demonstrate their newly learned cognitive skill of maintaining high sustained attention state levels and its challenge level is medium.

A score on a scale of 1-100% is calculated for each cognitive skill measured in Mission 3, according to the formulae set forth in Example 13. Specifically, Mission 3 includes calculation of a focused attention score, sustained attention score, cognitive inhibition score, and inner voice score.

At the end of Mission 3, a mission progress report is generated to show the details of the user's skills retention performance. A global attention score is calculated based on the user's performance with respect to attention scores. Additionally, a global composite score is calculated based on the user's performance with respect to attention and impulse/inhibition scores. The Mission 3 global attention score is indicative of the user's ability to focus and sustain his or her attention state levels. The completion of Mission 3 may mark the completion of the user's third training session, in which case the Mission 3 global attention score becomes the third data point on Focused/Sustained Attention graph of the user's summary performance report (see, for example, FIG. 23, top chart), and the Mission 3 global composite score becomes the third data point on the user's Composite of All Scores graph of the summary performance report (see, for example, FIG. 23, bottom chart).

Mission 4

Mission 4 begins with the introduction of “Smogbots.” Flying robots appear above the path, one at a time. The user must compare the characteristics of the robots to a sample, and tap the robots that match the sample. Robots that do not match the sample should not be selected. The user should not tap robots before they come in range.

The peer character provides the user with instructions for controlling their impulsivity, and a memorable phrase that can be repeated in order to internalize those instructions, “Wait 'till you know, then go.” The peer character explains how the Power Meter now responds to the player's correct and incorrect interactions with Smogbots. These Smogbot interactions are now the most critical factors to a player's progress in successfully completing the adventure story and each of its following missions. When the user makes multiple incorrect selections or rejections of Smogbots, the peer character provides additional encouragements and instructional cognitive skill reminders.

This segment trains behavioral inhibition, as measured by a user's ability to correctly select and reject Smogbots (see Example 13 for details of the behavioral inhibition score). Correct rejections are weighted more highly than incorrect rejections in the calculation. The challenge level is low.

In the skill transfer module, the user is prompted to correctly demonstrate the rejection of a molecule by its shape. When monitoring the screen for the appearance of molecules, the user must only tap molecules that match the shape of a specific set of examples. Tapping molecules that do not match will cause the user to recognize the negative consequence of incorrect commissions and/or omission and lose points toward the goal. Behavioral inhibition is measured by the number of correct molecules decoded. The challenge level of the skill transfer module is low.

A score on a scale of 1-100% is calculated for each cognitive skill measured in Mission 4, according to the formulae set forth in Example 13. Specifically, Mission 4 includes calculation of a focused attention score, sustained attention score, cognitive inhibition score, inner voice score, behavioral inhibition score, delay of gratification score, motivational inhibition score, and self-regulation score.

At the end of Mission 4, a mission progress report is generated to show the details of the user's skill performance. A global attention score is calculated based on the user's performance with respect to attention scores. Additionally, a global composite score is calculated based on the user's performance with respect to attention and impulse/inhibition scores. The completion of Mission 4 may mark the completion of the user's fourth training session, in which case the Mission 4 global attention score becomes the fourth data point on the user's Focused/Sustained Attention graph of the summary performance report (see, for example, FIG. 23, top chart), and the Mission 4 global composite score becomes the fourth data point on the user's Composite of All Scores graph of the summary performance report (see, for example, FIG. 23, bottom chart).

Mission 5

The skill training module of Mission 5 trains behavioral inhibition. In Mission 5, the challenge level is medium in both the skill training module and the skill transfer module.

A score on a scale of 1-100% is calculated for each cognitive skill measured in Mission 5, according to the formulae set forth in Example 13. Specifically, Mission 5 includes calculation of a focused attention score, sustained attention score, cognitive inhibition score, inner voice score, behavioral inhibition score, delay of gratification score, motivational inhibition score, and self-regulation score.

At the end of Mission 5, a mission progress report is generated to show the details of the user's cognitive skills demonstrated performance. A global attention score is calculated based on the user's skills performance with respect to attention scores. Additionally, a global composite score is calculated based on the user's performance with respect to attention and impulse/inhibition scores. The completion of Mission 5 may mark the completion of the user's fifth training session, in which case the Mission 5 global attention score becomes the fifth data point on the user's Focused/Sustained Attention graph of the summary performance report (see, for example, FIG. 23, top chart), and the Mission 5 global composite score becomes the fifth data point on the user's Composite of All Scores graph of the summary performance report (see, for example, FIG. 23, bottom chart).

Mission 6

The skill training module of Mission 6 trains behavioral inhibition. In Mission 6, the challenge level is high in both the skill training module and the skill transfer module.

A score on a scale of 1-100% is calculated for each cognitive skill measured in Mission 6, according to the formulae set forth in Example 13. Specifically, Mission 6 includes calculation of a focused attention score, sustained attention score, cognitive inhibition score, inner voice score, behavioral inhibition score, delay of gratification score, motivational inhibition score, and self-regulation score.

At the end of Mission 6, a mission progress report is generated to show the details of the user's cognitive skills performance. A global attention score is calculated based on the user's performance with respect to attention scores. Additionally, a global composite score is calculated based on the user's performance with respect to attention and impulse/inhibition scores. The completion of Mission 6 may mark the completion of the user's sixth training session, in which case the Mission 6 global attention score becomes the sixth data point on the user's Focused/Sustained Attention graph of the summary performance report (see, for example, FIG. 23, top chart), and the Mission 6 global composite score becomes the sixth data point on the user's Composite of All Scores Control graph of the summary performance report (see, for example, FIG. 23, bottom chart).

Mission 7

In Mission 7, Smogbot Groups are introduced. Flying robots appear above the path in groups of 2-4. The user must compare the characteristics of each robot in the group to a sample, and tap only the robots that match the sample. Groups only contain between 0 and 1 robots that match the sample. Robots that do not match the sample should not be selected.

The peer character provides the user with guidance for focusing in on salient details to enable skills development for, selective attention, delayed gratification and self-regulation. The peer character provides a memorable phrase that can be repeated in order to internalize those instructions, “Scan through and aim true.” When the user makes multiple incorrect selections or rejections of Smogbots that fly in groups, the peer character provides additional encouragements and guidance reminders.

Selective attention is measured by the user's ability to correctly select and reject Smogbots (see Example 13 for details of the selective attention score). In the calculation of this measurement, each Smogbot group represents a single interaction (e.g. when a user selects all Smogbots in a group of four, where one Smogbot matched the target and three did not, that interaction is considered a single incorrect action). The challenge level in Mission 7 is low.

The skill transfer module introduces molecule shape and color rejection. When monitoring the screen for the appearance of molecules, the user must only tap molecules that match the shape and color of an example. Tapping molecules that do not match both characteristics causes the user to loose points toward the goal. Selective attention is measured by the number of correct molecules decoded. The challenge level is low.

A score on a scale of 1-100% is calculated for each cognitive skill measured in Mission 7, according to the formulae set forth in Example 13. Specifically, Mission 7 includes calculation of a focused attention score, sustained attention score, cognitive inhibition score, inner voice score, interference control score, delay of gratification score, motivational inhibition score, self-regulation score, and selective attention score.

At the end of Mission 7, a mission progress report is generated to show the details of the user's performance. A global attention score is calculated based on the user's performance with respect to attention scores. Additionally, a global composite score is calculated based on the user's performance with respect to attention and impulse/inhibition scores. The completion of Mission 7 may mark the completion of the user's sixth training session, in which case the Mission 7 global attention score becomes the seventh data point on the user's Focused/Sustained Attention graph of the summary performance report (see, for example, FIG. 23, top chart), and the Mission 7 global composite score becomes the seventh data point on the user's Composite of All Scores graph of the summary performance report (see, for example, FIG. 23, bottom chart).

Mission 8

The skill training module of Mission 8 trains selective attention, delayed gratification and self-regulation. In Mission 8, the challenge level is medium in both the skill training module and the skill transfer module.

A score on a scale of 1-100% is calculated for each cognitive skill measured in Mission 8, according to the formulae set forth in Example 13. Specifically, Mission 8 includes calculation of a focused attention score, sustained attention score, cognitive inhibition score, inner voice score, interference control score, delay of gratification score, motivational inhibition score, self-regulation score, and selective attention score.

At the end of Mission 8, a mission progress report is generated to show the details of the user's demonstrated skills performance. A global attention score is developed based on the user's performance with respect to attention scores. Additionally, a global composite score is developed based on the user's performance with respect to attention and impulse/inhibition scores. The completion of Mission 8 may mark the completion of the user's eighth training session, in which case the Mission 8 global attention score becomes the eighth data point on the user's Focused/Sustained Attention graph of the summary performance report (see, for example, FIG. 23, top chart), and the Mission 8 global composite score becomes the eighth data point on the user's Composite of All Scores graph of the summary performance report (see, for example, FIG. 23, bottom chart).

Mission 9

The skill training module of Mission 9 trains selective attention, delayed gratification and self-regulation. In Mission 9, the challenge level is high in both the skill training module and the skill transfer module.

A score on a scale of 1-100% is calculated for each cognitive skill measured in Mission 9, according to the formulae set forth in Example 13. Specifically, Mission 9 includes calculation of a focused attention score, sustained attention score, cognitive inhibition score, inner voice score, interference control score, delay of gratification score, motivational inhibition score, self-regulation score, and selective attention score.

At the end of Mission 9, a mission progress report is generated to show the details of the user's demonstrated skills performance. A global attention score is developed based on the user's performance with respect to attention scores. Additionally, a global composite score is calculated based on the user's performance with respect to attention and impulse/inhibition scores. The completion of Mission 9 may mark the completion of the user's ninth training session, in which case the Mission 9 global attention score becomes the ninth data point on the user's Focused/Sustained Attention States graph of the summary performance report (see, for example, FIG. 23, top chart), and the Mission 9 global composite score becomes the ninth data point on the user's Composite of All Scores graph of the summary performance report (see, for example, FIG. 23, bottom chart).

Mission 10

In Mission 10, the Smogbot Dimension Switch is introduced. Flying robots appear above the path one at a time. The user must compare the characteristics of each robot in the group to a sample, and tap only the robots that match a single dimension (shape or color) of the sample. Periodically and unpredictably the sample and the salient dimension will switch (e.g. from shape to color). Robots that do not match the salient characteristic of the sample should not be selected.

The peer character provides the user with guidance for exercising behavioral inhibition, and a memorable phrase that can be repeated in order to internalize those instructions, “Adapt and excel.” Corrective voice-over guidance is now delivered by the user avatar, as a demonstration that the user must develop their own “inner voice” and not rely solely on external guidance. When the user makes multiple incorrect selections or rejections of Smogbots when target dimensions switch, the user avatar provides additional encouragements and guidance reminders to him or herself.

The user's ability to correctly select and reject Smogbots is measured as alternating attention (see Example 13 for details of the alternating attention score). In the calculation of this measurement, Smogbot encounters occurring immediately after a sample/dimension switch are weighted more highly. The challenge level is low.

In the skill transfer module, the molecule dimension switch is introduced. When monitoring the screen for the appearance of molecules, the user must only tap molecules that match the salient dimension of an example: its shape or color. The example and its salient dimension periodically change. Tapping molecules that do not match the correct dimension of the example causes the user to recognize the negative consequence of the incorrect commission and lose points toward the goal. Alternating attention is measured by number of correct molecules decoded. The challenge level is low.

A score on a scale of 1-100% is calculated for each cognitive skill measured in Mission 10, according to the formulae set forth in Example 13. Specifically, Mission 10 includes calculation of a focused attention score, sustained attention score, cognitive inhibition score, inner voice score, delay of gratification score, motivational inhibition score, and self-regulation score.

At the end of Mission 10, a mission progress report is generated to show the details of the user's performance. A global attention score is calculated based on the user's performance with respect to attention scores. Additionally, a global composite score is developed based on the user's performance with respect to attention and impulse/inhibition scores. The completion of Mission 10 may mark the completion of a training session, in which case the Mission 10 global attention score becomes a data point on the user's Focused/Sustained Attention graph of the summary performance report (see, for example, FIG. 23, top chart), and the Mission 10 global composite score becomes a data point on the user's Composite of All Scores graph of the summary performance report (see, for example, FIG. 23, bottom chart).

Mission 11

The skill training module of Mission 11 trains alternating attention, delayed gratification and self-regulation. In Mission 11, the challenge level is medium in both the skill training module and the skill transfer module.

A score on a scale of 1-100% is calculated for each cognitive skill measured in Mission 11, according to the formulae set forth in Example 13. Specifically, Mission 11 includes calculation of a focused attention score, sustained attention score, cognitive inhibition score, inner voice score, delay of gratification score, motivational inhibition score, and self-regulation score.

At the end of Mission 11, a mission progress report is generated to show the details of the user's performance. A global attention score is calculated based on the user's performance with respect to attention scores. Additionally, a global composite score is calculated based on the user's performance with respect to attention and impulse/inhibition scores. The completion of Mission 11 marks the completion of a training session, in which case the Mission 11 global attention score becomes a data point on the user's Focused/Sustained Attention States graph of the summary performance report (see, for example, FIG. 23, top chart), and the Mission 11 global composite score becomes a data point on the user's Composite of All Scores graph of the summary performance report (see, for example, FIG. 23, bottom chart).

Mission 12

The skill training module of Mission 12 trains alternating attention, delayed gratification and self-regulation. In Mission 12, the challenge level is high in both the skill training module and the skill transfer module.

A score on a scale of 1-100% is calculated for each cognitive skill measured in Mission 12, according to the formulae set forth in Example 13. Specifically, Mission 12 includes calculation of a focused attention score, sustained attention score, cognitive inhibition score, inner voice score, delay of gratification score, motivational inhibition score, and self-regulation score.

At the end of Mission 12, a mission progress report is generated to show the details of the user's performance. A global attention score is calculated based on the user's performance with respect to attention scores. Additionally, a global composite score is calculated based on the user's performance with respect to attention and impulse/inhibition scores. The completion of Mission 12 may mark the completion of a training session, in which case the Mission 12 global attention score becomes a data point on the user's Focused/Sustained Attention graph of the summary performance report (see, for example, FIG. 23, top chart), and the Mission 12 global composite score becomes a data point on the user's Composite of All Scores graph of the summary performance report (see, for example, FIG. 23, bottom chart).

Mission 13

Mission 13 contains new environmental distractors not seen in missions 1-12, Smogbots with characteristics not seen in previous missions, and frequent background “radio chatter” voice-over as distractions. Additionally, Teach-To-Learn is introduced. Corrective guidance is now delivered by the user avatar character towards a “trainee” character, as a demonstration that an effective way to master a skill is to teach it to another person.

Behavioral Inhibition and Novelty Inhibition are measured by a user's ability to correctly select and reject Smogbots despite new distractions. The challenge level is high.

The skill transfer module includes lab novelty distractors. The transfer environment is punctuated by loud noises and distracting visual effects. Behavioral inhibition and novelty inhibition are measured by the number of correct molecules decoded despite new distractions (see Example 13 for details of behavioral inhibition and novelty inhibition scores). The challenge level is high.

A score on a scale of 1-100% is calculated for each cognitive skill measured in Mission 13, according to the formulae set forth in Example 13. Specifically, Mission 13 includes calculation of a focused attention score, sustained attention score, cognitive inhibition score, behavioral inhibition score, novelty inhibition score, delay of gratification score, inner voice score, motivational inhibition score, and self-regulation score.

At the end of Mission 13, a mission progress report is generated to show the details of the user's performance. A global attention score is calculated based on the user's performance with respect to attention scores. Additionally, a global composite score is developed based on the user's performance with respect to attention and impulse/inhibition scores. The completion of Mission 13 marks the completion of a training session, in which case the Mission 13 global attention score becomes a data point on the user's Focused/Sustained Attention States graph of the summary performance report (see, for example, FIG. 23, top chart), and the Mission 13 global composite score becomes a data point on the user's Composite of All Scores graph of the summary performance report (see, for example, FIG. 23, bottom chart).

Mission 14

The skill training module of Mission 14 trains selective attention, novelty inhibition, delayed gratification and self-regulation, as measured by a user's ability to correctly select and reject Smogbots in groups despite new distractions. In Mission 14, the challenge level is high in both the skill training module and the skill transfer module.

A score on a scale of 1-100% is calculated for each cognitive skill measured in Mission 14, according to the formulae set forth in Example 13. Specifically, Mission 14 includes calculation of a focused attention score, sustained attention score, cognitive inhibition score, selective attention score, interference control score, novelty inhibition score, delay of gratification score, inner voice score, motivational inhibition score, and self-regulation score.

At the end of Mission 14, a mission progress report is generated to show the details of the user's performance. A global attention score is developed based on the user's performance with respect to attention scores. Additionally, a global composite score is calculated based on the user's performance with respect to attention and impulse/inhibition scores. The completion of Mission 14 may mark the completion of a training session, in which case the Mission 14 global attention score becomes a data point on the user's Focused/Sustained Attention States graph of the summary performance report (see, for example, FIG. 23, top chart), and the Mission 14 global composite score becomes a data point on the user's Composite of All Scores graph of the summary performance report (see, for example, FIG. 23, bottom chart).

Mission 15

The skill training module of Mission 15 trains alternating attention, novelty inhibition, delayed gratification and self-regulation, as measured by a user's ability to correctly select and reject Smogbots when the salient dimension of the target switches, despite new distractions.

In the skill transfer module of Mission 15, alternating attention and novelty inhibition are measured by the number of correct molecules are decoded in spite of new distractions. The challenge level is high.

A score on a scale of 1-100% is calculated for each cognitive skill measured in Mission 15, according to the formulae set forth in Example 13. Specifically, Mission 15 includes calculation of a focused attention score, sustained attention score, cognitive inhibition score, alternating attention score, divided attention score, novelty inhibition score, delay of gratification score, inner voice score, motivational inhibition score, and self-regulation score.

At the end of Mission 15, a mission progress report is generated to show the details of the user's performance. A global attention score is developed based on the user's performance with respect to attention scores. Additionally, a global composite score is developed based on the user's performance with respect to all attention and impulse/inhibition scores. The completion of Mission 15 may mark the completion of a training session, in which case the Mission 15 global attention score becomes a data point on the user's Focused/Sustained Attention States graph of the summary performance report (see, for example, FIG. 23, top chart), and the Mission 15 global composite score becomes a data point on the user's Composite of All Scores graph of the summary performance report (see, for example, FIG. 23, bottom chart).

In any of the preceding mission summaries, multiple missions may have been completed in one training session. In this case, a summary performance report may include multiple data points for a single training session, or a composite score (e.g., an average or a weighted average) can be derived from the multiple data points for that training session.

A summary progress report generated as described above can be used to determine a change in each attention score and/or a change in a global attention score over the period of training. Similarly, a change in each impulse/inhibition score and/or a change in a global impulse/inhibition score can be determined for a subject over the period of training. This change can be determined by any suitable means (e.g., taking the difference between the first global score and the last global score; or fitting a line to the data (e.g., a linear or non-linear, e.g., curvilinear line) and taking the difference between its Y intercept and its Y value at the last data point).

### Example 16. Case Example

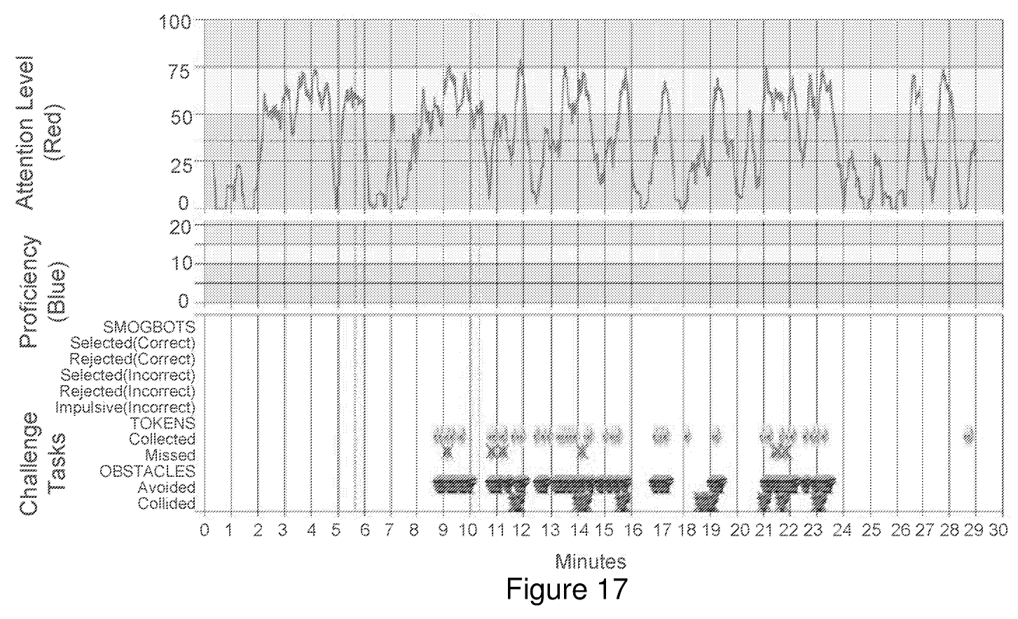

This example presents a case of a user who underwent 6 weeks of training and completed all 15 missions. The following description and accompanying figures (FIGS. 17-23) provides mission-by-mission and overall analysis of the subject's performance over the period of training. The subject played the game described in Example 15, which trains and measures each cognitive skill score and global score as described in Example 13 and 14, respectively.

Mission 2

Mission 2 is an exemplary mission of the first level. As shown in FIG. 17, the user was presented with collision avoidance (obstacles) and collection (crystal) challenge tasks as part of the skill training module, and the results of the challenge tasks are shown in the MPR. The user passed the mission with a global attention score of 35.7. In the skill transfer module, the user repeated performed a fog analysis to demonstrate retention of cognitive skills trained, and proceeded to the next level when they reached the target.

Mission 4

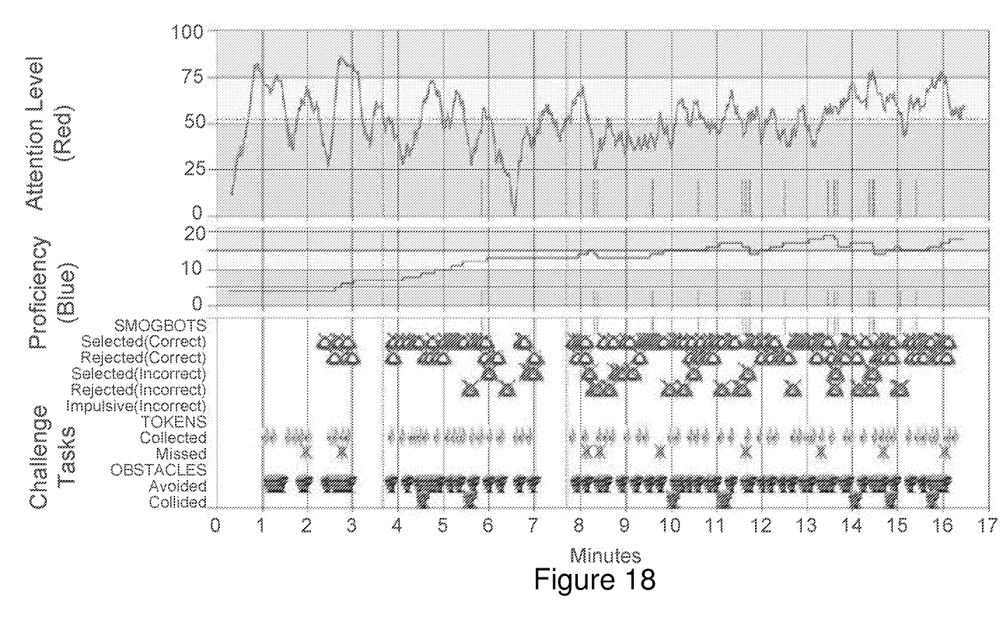

Mission 4 is an exemplary mission of the second level. As shown in FIG. 18, the user was presented with additional challenge tasks, “Smogbots.” Correctly selected, correctly rejected, incorrectly rejected, and correctly rejected Smogbots are depicted over the course of the skill training module in the MPR. The user's increasing attention state level over time is depicted in the Attention Level panel of the MPR. The user passed the mission with a global attention score of 52.3. Upon completion of the mission, the user advanced to the skill transfer module.

Mission 8

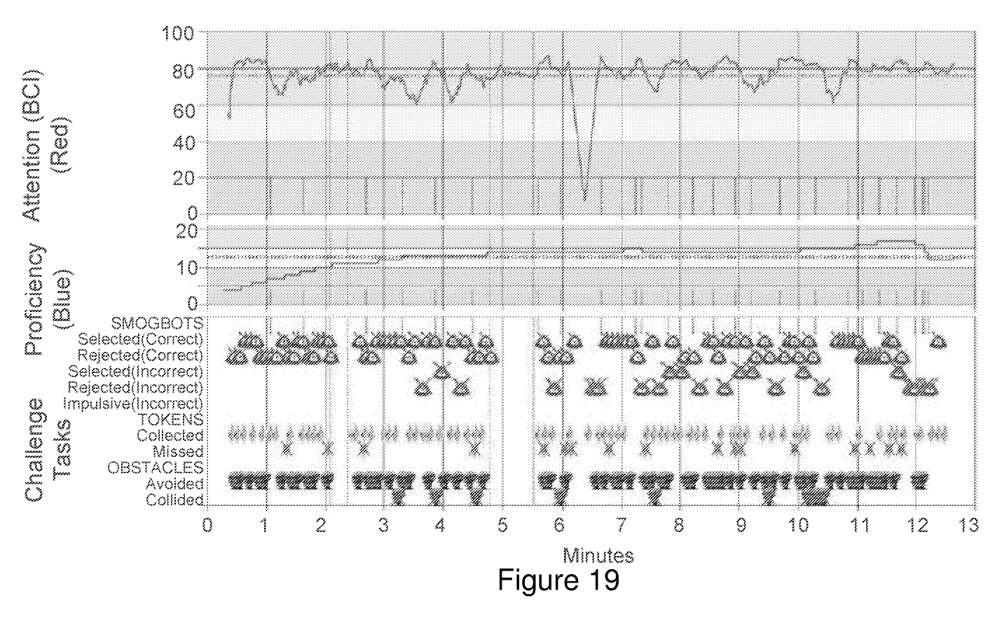

Mission 8 is an exemplary mission of the third level. As shown in FIG. 19, the user demonstrated high attention state levels throughout the training module and passed the mission with a global attention score of 76. Upon completion of the mission, the user advanced to the skill transfer module.

Mission 12

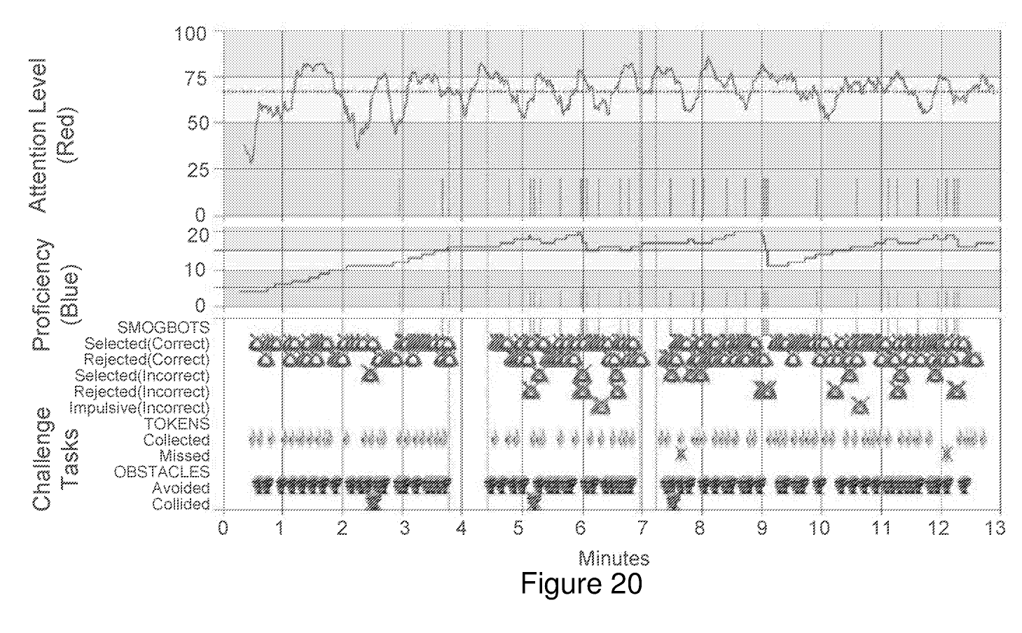

Mission 12 is an exemplary mission of the fourth level. As shown in FIG. 20, the user demonstrated high attention state levels throughout the training module. In Mission 12, the user's impulsive responses to impulse/inhibition challenge tasks are shown as markers along the “impulsive (incorrect)” row of the challenge task panel of the MPR. The user passed the training module with a global attention score of 67.2. The user proceeded to the skills transfer module, where another fog analysis was performed.

Mission 14

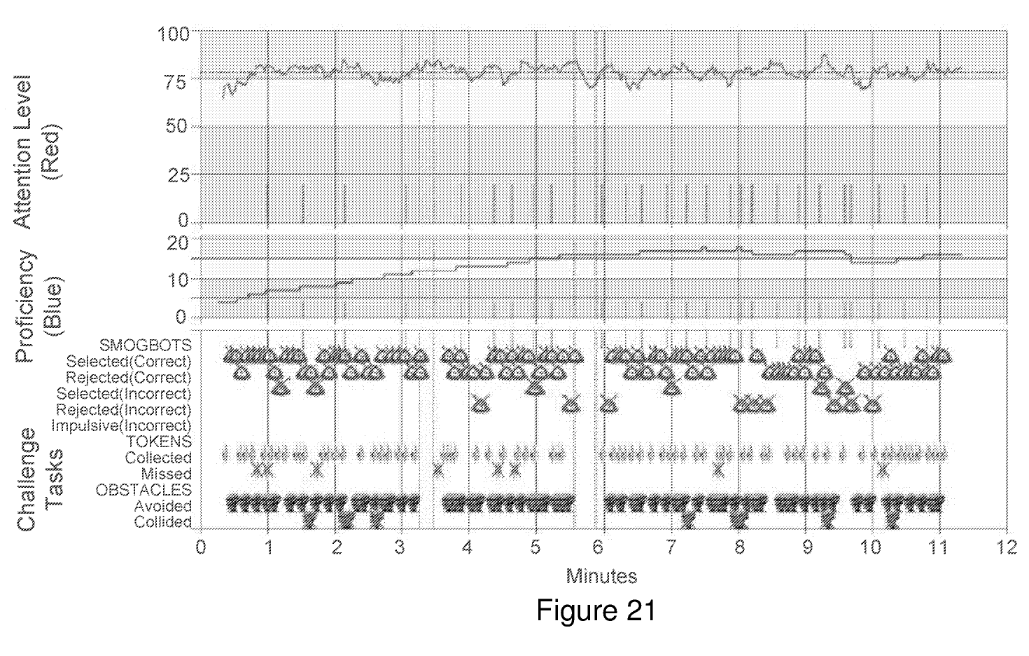

Mission 14 introduced new distractions to the user to reinforce the ability to reject higher levels of distractions and stay on task. As shown in FIG. 21, the user maintained an exceptional attention state level throughout the majority of the skill training module. The user passed the mission with a global attention score of 78.2.

Mission 15

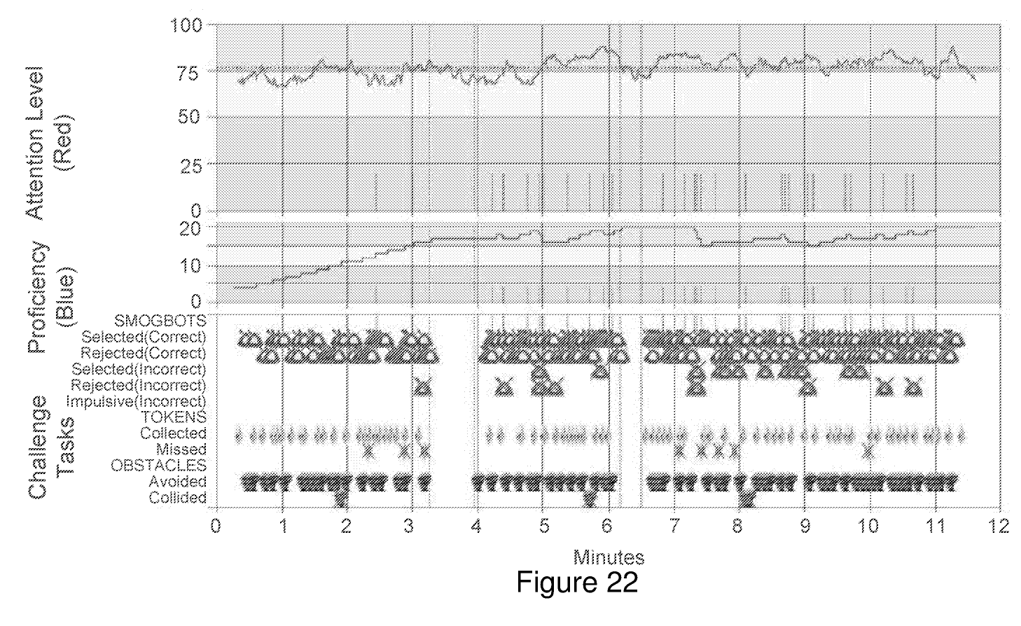

Mission 15 was the last mission of the period of training. Similar to mission 14, the user maintained an exceptional attention state level throughout the majority of the skill training module, as shown in FIG. 22. The user passed the mission with a global attention score of 76.9.

FIG. 23 shows a summary progress report for the user of the course of his training period. Prior to undergoing the period of training, the user had an ADHD-RS score of 29 (Moderately severe ADHD), which reduced to 11 (normal non ADHD behavior) after training, marking a 62% improvement. The user's global attention score increased by 90%, from 42 to 80.

### Example 17. Training History and Training Planner

The methods and systems of the invention can include a report generated for a physician or parent describing the number of sessions and/or length of sessions undertaken by the user. The methods and systems of the invention can also include a training planner equipped with a calendar for scheduling training sessions and providing a user with a reminder of the scheduled training sessions.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method for training a cognitive skill in a user, the method comprising:
(a) providing a computer-based virtual learning curriculum configured to train a cognitive skill in the user, wherein the virtual learning curriculum comprises at least a first game module and a second game module, wherein the first game module comprises a skill training module for training a targeted cognitive skill and the second game module comprises a skill transfer module configured to permit the user to demonstrate retention of the targeted cognitive skill in a virtual environment outside the skill training module;
(b) measuring the EEG brain activity signals of the user and on the basis of the EEG brain activity signals calculating the attention state level of the user;
(c) performing a training exercise in the skill training module, the skill training module comprising a first story line for advancing a user avatar toward completion of a mission while eliciting high attention state levels in the user, wherein an increase or decrease in the attention state level of the user produces a corresponding increase or decrease in the speed of the user avatar towards the completion of the mission;
(d) during step (c), presenting challenge tasks to the user, wherein the challenge tasks are configured to train a targeted cognitive skill in the user;
(e) during step (d), on the basis of the user response to the challenge tasks, calculating a skill performance score for the user and increasing the difficulty of achieving the challenge tasks when the skill performance score rises above a predetermined upper threshold and decreasing the difficulty of achieving the challenge tasks when the skill performance score falls below a predetermined lower threshold while the user avatar advances towards the completion of the mission; and
(f) following completion of the mission, performing a skill retention exercise in the skill transfer module, the skill transfer module comprising a second story line for presenting retention challenge tasks to the user, wherein the retention challenge tasks are different from the challenge tasks presented in skill training module, wherein the retention challenge tasks are configured for the user to demonstrate retention of the targeted cognitive skill.

2. The method of claim 1, wherein the first story line comprises a peer character presented to provide guidance and motivation to the user to develop an inner voice in the user.

3. The method of claim 2, wherein the peer character dynamically provides guidance and motivation to the user to learn targeted cognitive skills while providing self-esteem or encouragement to develop an inner voice in the user.

4. The method of any one of claim 1 or 2, wherein the first story line and the second story line comprise a mentor character configured to encourage the user to engage in problem solving, and be self-motivated to develop an inner voice in the user.

5. The method of claim 4, wherein the mentor character is not configured to demonstrate the challenge task to the user.

6. The method of any one of claims 1-5, wherein step (e) comprises adjusting the difficulty of achieving the challenge tasks based upon the skill performance score of the user.

7. The method of claim 6, wherein step (e) comprises adjusting the difficulty of achieving the challenge tasks based upon both the skill performance score and the attention state level of the user.

8. The method of any one of claims 1-7, wherein step (e) further comprises adjusting the order of the targeted cognitive skills presented to the user avatar based upon the skill performance score or the attention state level of the user.

9. The method of any one of claims 1-8, wherein the speed of a user avatar increases with increases in the skill performance score.

10. The method of any one of claims 1-9, wherein the speed of a user avatar decreases with decreases in the skill performance score.

11. The method of any one of claims 1-10, wherein step (d) comprises presenting challenge tasks to the user avatar at a rate that increases when the attention state level of the user increases.

12. The method of any one of claims 1-11, wherein step (d) comprises presenting challenge tasks to the user avatar at a rate that decreases when the attention state level of the user decreases.

13. The method of any one of claims 1-12, wherein step (d) comprises presenting at least some challenge tasks to the user avatar only after the user has reached a predetermined attention state level.

14. The method of any one of claims 1-13, wherein step (f) further comprises, on the basis of the user avatar response to the challenge tasks presented in the skill transfer module, calculating a skill transfer score for the user, wherein achieving a skill transfer score above a predetermined threshold demonstrates transferability of the retained targeted cognitive skill and permits the user avatar to advance to the next level of the computer-based virtual learning curriculum.

15. The method of any one of claims 1-14, wherein the skill training module is configured to train attention maintenance and the skill transfer module is configured for the user to demonstrate retention of the skill of attention maintenance.

16. The method of claim 15, further comprising, following completion of the mission, calculating a focused attention score.

17. The method of claim 16, wherein the focused attention score is calculated from the number of attention state levels above a predetermined threshold attention state level.

18. The method of claim 17, wherein the predetermined threshold attention state level is greater than 50%, 55%, 60%, 65%, 70%, 75%, 80%, or 90%.

19. The method of claim 15, further comprising, following completion of the mission, calculating a sustained attention score.

20. The method of claim 19, wherein the sustained attention score is calculated from a duration of time during which attention state levels vary by less than a predetermined threshold variance.

21. The method of claim 20, wherein the predetermined threshold variance is between 1% and 50% of the preceding attention state level.

22. The method of claim 20 or 21, wherein the sustained attention score is calculated for sequential attention state levels greater than a predetermined threshold attention state level.

23. The method of any one of claims 1-22, wherein the skill training module is configured to train cognitive inhibition and the skill transfer module is configured for the user to demonstrate retention of the skill of cognitive inhibition.

24. The method of claim 23, further comprising, following completion of the mission goal, calculating a cognitive inhibition score.

25. The method of claim 24, wherein the cognitive inhibition score is calculated from the frequency of attention state levels over a predetermined threshold attention state level for a period of time following the beginning of step (c).

26. The method of claim 25, wherein the predetermined threshold attention state level is 50%, 55%, 60%, 65%, 70%, 75%, 80%, or 90%.

27. The method of any one of claims 1-26, wherein the skill training module is configured to train behavioral inhibition and the skill transfer module is configured for the user to demonstrate retention of the skill of behavioral inhibition.

28. The method of claim 27, further comprising:
(a) following completion of the mission, determining (i) a number of correctly rejected challenge tasks; and (ii) a number of incorrectly selected challenge tasks; and
(b) calculating a behavioral inhibition score from a composite of (i) and (ii).

29. The method of any one of claims 1-28, wherein the skill training module is configured to train selective attention and the skill transfer module is configured for the user to demonstrate retention of the skill of selective attention.

30. The method of claim 29, further comprising:
(a) following completion of the mission, determining (i) a number of correctly selected challenge tasks; (ii) a number of correctly rejected challenge tasks; and (iii) a total number of challenge tasks;
(b) calculating a selective attention score from a composite of (i)-(iii).

31. The method of any one of claims 1-30, wherein the skill training module is configured to train alternating attention and the skill transfer module is configured for the user to demonstrate retention of the skill of alternating attention.

32. The method of claim 31, further comprising:
(a) following completion of the mission, determining (i) a number of correctly selected challenge tasks; and (ii) a number of correctly rejected challenge tasks, wherein the challenge tasks are presented immediately after a target rule switch; and
(b) calculating an alternating attention score from a composite of (i) and (ii).

33. The method of any one of claims 1-32, wherein the skill training module is configured to train novelty inhibition and the skill transfer module is configured for the user to demonstrate retention of the skill of novelty inhibition.

34. The method of claim 33, further comprising:
(a) following completion of the mission, determining (i) a number of correctly selected challenge tasks; (ii) a number of correctly rejected challenge tasks; and (iii) a total number of challenge tasks; and
(b) calculating a novelty inhibition score from a composite of (i)-(iii).

35. The method of any one of claims 1-34, wherein the skill training module is configured to train delay of gratification and the skill transfer module is configured for the user to demonstrate retention of the skill of delay of gratification.

36. The method of claim 35, further comprising:
(a) following completion of the mission, determining (i) a number of correctly selected challenge tasks; and (ii) a total number of challenge tasks; and
(b) calculating a delay of gratification score from a composite of (i) and (ii).

37. The method of any one of claims 1-36, wherein the skill training module is configured to train self-regulation and the skill transfer module is configured for the user to demonstrate retention of the skill of self-regulation.

38. The method of claim 37, further comprising:
(a) following completion of the mission, determining (i) a number of correctly selected challenge tasks; and (ii) a total number of challenge tasks; wherein the challenge tasks occur within a predetermined time before or after a collection or collision avoidance challenge task; and
(b) calculating a self-regulation score from a composite of (i) and (ii).

39. The method of any one of claims 1-38, wherein the skill training module is configured to train divided attention and the skill transfer module is configured for the user to demonstrate retention of the skill of divided attention.

40. The method of claim 39, further comprising:
(a) following completion of the mission, determining (i) a number of correctly selected challenge tasks; (ii) a number of correctly rejected challenge tasks; (iii) a total number of challenge tasks; and
(b) calculating a divided attention score from a composite of (i)-(iii).

41. The method of any one of claims 1-40, wherein the skill training module is configured to train interference control and the skill transfer module is configured for the user to demonstrate retention of the skill of interference control.

42. The method of claim 41, further comprising:
(a) following completion of the mission, determining (i) a number of incorrectly selected challenge tasks; and (ii) a total number of challenge tasks; and
(b) calculating an interference control score from a composite of (i) and (ii).

43. The method of any one of claims 1-42, wherein the skill training module is configured to train motivational inhibition and the skill transfer module is configured for the user to demonstrate retention of the skill of motivational inhibition.

44. The method of claim 43, further comprising:
(a) following completion of the mission, determining (i) a number of correctly selected challenge tasks occurring after an incorrectly selected or an incorrectly rejected challenge task; (ii) a number of correctly rejected challenge tasks occurring after an incorrectly selected or an incorrectly rejected challenge task; (iii) a total number of correctly selected challenge tasks; (iv) a total number of correctly rejected challenge tasks; (v) a number of incorrectly selected challenge tasks; and (vi) a number of incorrectly rejected challenge tasks;
(b) calculating a motivational inhibition score from a composite of (i)-(vi).

45. The method of any one of claims 1-44, wherein the skill training module is configured to train inner voice and the skill transfer module is configured for the user to demonstrate retention of the skill of inner voice.

46. The method of claim 45, wherein an inner voice score is calculated, following completion of the mission, from a number of attention state levels greater than a preceding attention state level, wherein the preceding attention state level is less than a predetermined threshold level.

47. The method of any one of claims 1-46, wherein the skill transfer module is configured to enable the user to demonstrate retention of the targeted cognitive skill.

48. The method of claim 47, wherein the demonstration of retention corresponds to an increased chance of achievement of the desired goal of the challenge tasks or an increase in targeted cognitive skill learning.

49. The method of any one of claims 1-48, wherein the user has low attention and/or inhibition control.

50. The method of any one of claims 1-49, wherein the user has an inattention or inhibition disorder.

51. The method of any one of claims 1-50, further comprising analyzing and reporting the skills performance of the user.

52. The method of claim 51, wherein the skills performance is a targeted cognitive skills performance.

53. The method of any of claims 1-52, wherein the modules are comprised of one or more levels, each level optionally being comprised of one or more missions.

54. The method of claim 53, wherein the one or more levels are configured for the development of one or more targeted cognitive skills.

55. The method of claim 54, wherein the targeted cognitive skills are selected from focused attention, sustained attention, cognitive inhibition, behavioral inhibition, selective attention, alternating attention, divided attention, interference control, novelty inhibition, delay of gratification, inner voice, motivational inhibition, and self-regulation.

56. The method of any one of claims 1-55, wherein steps (a) to (f) are repeated for at least one targeted cognitive skill.

57. The method of any one of claims 1-56, wherein steps (a) to (f) are repeated for two or more targeted cognitive skills.

58. The method of claim 56 or 57, wherein the method is performed at regular intervals such as 3, 4, 5, 6, or 7 times per week for 10, 20, 30, 40, 50, or 60 minutes, over a course of 3 or more weeks to train targeted cognitive skills of the user.

59. The method of any of claims 1-58, wherein the skill training module comprises (i) providing a score of a user's skill performance, and (ii) on the basis of the score, selecting a difficulty level for the skill training module.

60. The method of claim 59, wherein the user's skills performance is quantified by said user's accuracy in correctly distinguishing their activity between various stimuli.

61. The method of any one of claims 1-60, further comprising during step (d), on the basis of the responses, (i) identifying the impulsive responses by determining when the user is incorrectly responding to an impulse/inhibition challenge task or responding to a non-stimulus, and (ii) alerting the user to the impulsive responses.

62. The method of claim 61, wherein the alerting comprises presenting the user with an audio or visual cue when the impulsive responses are identified.

63. The method of claim 61 or 62, wherein the user is subjected to an immediate negative consequence when the impulsive responses are identified.

64. The method of any one of claim 59-63, wherein step (d) comprises calculating a skills performance score for the user on the basis of the user response to the challenge tasks, and step (e) comprises reducing the skills performance score when the impulsive responses are identified.

65. The method of claim 64, wherein the skills performance score is quantified using a combination of (i) the user accuracy in correctly distinguishing between various stimuli, and (ii) the ability of the user to inhibit impulsive responses.

66. The method of any one of claims 1-65, further comprising during step (d), identifying when the user is frustrated with anxiety and triggering voice-over dialog from a peer character or a mentor character.

67. The method of claim 66, wherein the peer character or the mentor character provides reassurance or simple strategies for regulating emotional responses to feelings of frustration.

68. A game based system for training a targeted cognitive skill in a user, the system comprising a processor equipped with an algorithm for presenting a computer-based virtual learning curriculum according to the methods of any one of claims 1-67.

69. The game based system of claim 68, wherein the algorithm is for presenting a computer-based virtual learning curriculum while the user is in a state of focused and/or sustained attention.

70. The game based system of claim 68 or 69, further comprising an EEG headset for collecting and communicating EEG data from the user to a computing and video device.

71. The method of any one of claims 1-67, the method further comprising:
(d) deriving an attention score for each of the attention-associated skills on the basis of the attention state level and/or the user response to the challenge task, wherein the attention-associated skills comprise focused attention, sustained attention, selective attention, alternating attention, or divided attention and deriving an attention or impulse/inhibition score for each of the attention- or impulse/inhibition-associated skills on the basis of the attention state and/or the user response to the challenge task, wherein the attention- or impulse/inhibition-associated skills comprise focused attention, sustained attention, cognitive inhibition, behavioral inhibition, selective attention, alternating attention, divided attention, interference control, novelty inhibition, delay of gratification, inner voice, motivational inhibition, or self-regulation;
(f) for each training session,
(i) calculating a global attention score derived from each of the attention scores; and/or
(ii) calculating a global composite score derived from each of the attention or impulse/inhibition scores; and
(g) determining, over the period of training,
(i) a change in each attention score and a change in the global attention score; or
(ii) a change in each attention and impulse/inhibition score and a change in the global composite score.

72. A method for training targeted cognitive skills in a user, the method comprising:
(a) over a period of training comprising multiple training sessions, providing a computer-based virtual learning curriculum configured to train a plurality of attention-associated skills;
(b) measuring the EEG brain activity signals of the user and on the basis of the EEG brain activity signals, calculating an attention state levels of the user;
(c) presenting a challenge task to the user, wherein the challenge task is configured to train one or more of the plurality of the attention-associated skills in the user;
(d) deriving an attention score for each of the attention-associated skills on the basis of the attention state level and/or the user response to the challenge task, wherein the attention-associated skills comprise focused attention, sustained attention, selective attention, alternating attention, or divided attention;
(e) for each training session, calculating a global attention score derived from each of the attention scores; and
(f) determining, over the period of training, a change in each attention score and/or a change in the global attention score.

73. A method for training targeted cognitive skills in a user, the method comprising:
(a) over a period of training comprising multiple training sessions, providing a computer-based virtual learning curriculum configured to train a plurality of attention-associated skills and impulse/inhibition-associated skills;
(b) measuring the EEG brain activity signals of the user and on the basis of the EEG brain activity signals, calculating an attention state level of the user;
(c) presenting a challenge task to the user, wherein the challenge task is configured to train one or more of the plurality of the attention-associated skills and impulse-inhibition-associated skills in the user;
(d) deriving an attention and impulse/inhibition score for each of the attention- and impulse/inhibition-associated skills on the basis of the attention state level and/or the user response to the challenge task, wherein the attention- and impulse/inhibition-associated skills comprise focused attention, sustained attention, cognitive inhibition, behavioral inhibition, selective attention, alternating attention, divided attention, interference control, novelty inhibition, delay of gratification, inner voice, motivational inhibition, or self-regulation;
(f) for each training session, calculating a global composite score derived from a composite of each of the attention and impulse/inhibition scores; and
(g) determining, over the period of training, a change in each attention and impulse/inhibition score and/or a change in the global attention and impulse/inhibition score.

74. The method of claim 72 or 73, comprising:
(a) following completion of the mission, determining a number of attention state levels above a predetermined threshold attention state level; and
(b) calculating a focused attention score from the number of attention state levels above the predetermined threshold attention state level.

75. The method of claim 74, wherein the predetermined threshold attention state level is 50%, 55%, 60%, 65%, 70%, 75%, 80%, or 90%.

76. The method of claim 72 or 73, comprising:
(a) following completion of the mission, determining a duration of time during which attention state levels vary by less than a predetermined threshold variance; and
(b) calculating a sustained attention score from the duration of time during which attention states levels vary by less than a predetermined threshold variance.

77. The method of claim 76, wherein the predetermined threshold variance is between 1% and 50% of the preceding attention state level.

78. The method of claim 77, wherein the sustained attention score is calculated for sequential attention states levels greater than a predetermined attention state level.

79. The method of claim 72 or 73, comprising:
(a) following completion of the mission, determining (i) a number of correctly selected challenge tasks; (ii) a number of correctly rejected challenge tasks; and (iii) a total number of challenge tasks; and
(b) calculating a selective attention score from a composite of (i)-(iii).

80. The method of claim 72 or 73, comprising:
(a) following completion of the mission, determining (i) a number of correctly selected challenge tasks; and (ii) a number of correctly rejected challenge tasks, wherein the challenge tasks are presented immediately after a target rule switch; and
(b) calculating an alternating attention score from a composite of (i) and (ii).

81. The method of claim 72 or 73, comprising:
(a) following completion of the mission, determining (i) a number of correctly selected challenge tasks; (ii) a number of correctly rejected challenge tasks; (iii) a total number of challenge tasks; and
(b) calculating a divided attention score from a composite of (i)-(iii).

82. The method of claim 72 or 73, comprising:
(a) following completion of the mission, determining a number of attention state levels over a predetermined threshold attention state level for a period of time following the beginning of step (c); and
(b) calculating a cognitive inhibition score from the number of attention state levels determined in part (a).

83. The method of claim 82, wherein the predetermined threshold attention state level is 50%, 55%, 60%, 65%, 70%, 75%, 80%, or 90%.

84. The method of claim 72 or 73, comprising:
(a) following completion of the mission, determining (i) a number of correctly rejected challenge tasks, and (ii) a number of incorrectly selected challenge tasks; and
(b) calculating a behavioral inhibition score based on (i) and (ii).

85. The method of claim 72 or 73, comprising:
(a) following completion of the mission, determining (i) a number of incorrectly selected challenge tasks; and (ii) a total number of challenge tasks; and
(b) calculating an interference control score from a composite of (i) and (ii).

86. The method of claim 72 or 73, comprising:
(a) following completion of the mission, determining (i) a number of correctly selected challenge tasks; (ii) a number of correctly rejected challenge tasks; and (iii) a total number of challenge tasks; and
(b) calculating a novelty inhibition score from a composite of (i)-(iii).

87. The method of claim 72 or 73, comprising:
(a) following completion of the mission, determining (i) a number of correctly selected challenge tasks occurring after an incorrectly selected or an incorrectly rejected challenge task; (ii) a number of correctly rejected challenge tasks occurring after an incorrectly selected or an incorrectly rejected challenge task; (iii) a total number of correctly selected challenge tasks; (iv) a total number of correctly rejected challenge tasks; (v) a number of incorrectly selected challenge tasks; and (vi) a number of incorrectly rejected challenge tasks; and
(b) calculating a motivational inhibition score from a composite of (i)-(vi).

88. The method of claim 72 or 73, comprising:
(a) following completion of the mission, determining a number of attention state levels greater than a preceding attention state level, wherein the preceding attention state level is less than a predetermined threshold attention state level; and
(b) calculating an inner voice score from the number of attention state levels in part (a).

89. The method of claim 88, wherein the predetermined threshold attention state level is 10%, 20%, 30%, 40%, 50%, 60%, or 70%.

90. The method of claim 88 or 89, wherein the attention state levels are each greater than the preceding attention state level by at least 10%, at least 20%, at least 30%, at least 40%, or at least 50%.

91. The method of claim 72 or 73, comprising:
(a) following completion of the mission, determining (i) a number of correctly selected challenge tasks; and (ii) a total number of challenge tasks; and
(b) calculating a delay of gratification score from a composite of (i) and (ii).

92. The method of claim 72 or 73, comprising:
(a) following completion of the mission, determining (i) a number of correctly selected challenge tasks; and (ii) a total number of challenge tasks; wherein the challenge tasks occur within a predetermined time following a collection or collision avoidance challenge task; and
(b) calculating a self-regulation score from a composite of (i) and (ii).

93. The method of claim 92, wherein the predetermined time is 0 to 5 seconds.

94. The method of claim 72, wherein the global attention score is a composite score of the attention scores.

95. The method of claim 73, wherein the global composite score is composite of the attention scores and the impulse/inhibition scores.

96. A method for treating an inattention disorder in a user in need thereof, the method comprising:
(a) providing a computer-based virtual learning curriculum configured to train a targeted cognitive skill in the user, wherein the virtual training environment comprises at least a first game module and a second game module, wherein the first game module comprises a skill training module for training a targeted cognitive skill and the second game module comprises a skill transfer module configured to permit the user to demonstrate retention of the targeted cognitive skill in a virtual environment outside the skill training environment;
(b) measuring the EEG brain activity signals of the user and on the basis of the EEG brain activity signals calculating the attention state level of the user;
(c) performing a training exercise in the skill training module, the skill training module comprising a first story line for advancing a user avatar toward completion of a mission while eliciting attention state levels in the user, wherein an increase or decrease in the attention state level of the user produces a corresponding increase or decrease in the speed of the user avatar;
(d) during step (c), presenting impulse/inhibition challenge tasks to the user to elicit responses from the user via an input device, wherein the impulse/inhibition challenge tasks are configured to train the targeted cognitive skill in the user;
(e) during step (d), on the basis of the responses, (i) identifying impulsive responses by determining when the user is impulsively responding, and (ii) alerting the user to the impulsive response; and
(f) following completion of the mission, performing a skill retention exercise in the skill transfer module, the skill transfer module comprising a second story line for presenting the challenge tasks to the user in a virtual learning curriculum from the skill training module, wherein the challenge tasks are configured for the user to demonstrate retention of the targeted cognitive skill learned in the training module.

97. The method of claim 96, wherein the attention state level of the user is scaled from 0% to 100%.

98. The method of claim 97, wherein step (e) further comprises (iii) adaptively providing similar challenge tasks to retrain a desired impulse inhibition.

99. The method of any one of claims 96-98, wherein the alerting comprises presenting the user with an audio or visual cue to an impulsive response.

100. The method of any one of claims 96-99, wherein step (d) comprises calculating a skills performance score for the user on the basis of the user response to the impulse/inhibition challenge tasks, and step (e) comprises reducing the skills performance score when the impulsive response are identified.

101. The method of any one of claims 96-100, wherein step (e) comprises on the basis of the user response to the challenge tasks, calculating a skills performance score for the user and increasing the difficulty of the challenge tasks when the skills performance score rises above a predetermined upper threshold and decreasing the difficulty of the challenge tasks when the skills performance score falls below a predetermined lower threshold while the user avatar advances towards the desired goal.

102. The method claim 101, wherein step (e) comprises adjusting the difficulty of achieving the impulse/inhibition challenge tasks based upon both the skills performance score and the attention state of the user.

103. The method of any one of claims 96-102, wherein step (d) comprises presenting impulse/inhibition challenge tasks to the user at a rate that increases when the attention state of the user increases.

104. The method of any one of claims 96-103, wherein step (d) comprises presenting impulse/inhibition challenge tasks to the user at a rate that decreases when the attention state level of the user decreases.

105. The method of any one of claims 96-104, wherein step (d) comprises presenting at least some impulse/inhibition challenge tasks to the user only after the user has reached a predetermined threshold attention state level.

106. The method of claim 105, wherein step (d) comprises presenting at least some challenge tasks to the user only after the user has reached a predetermined threshold attention state level and only while the user maintains an attention state level above the predetermined threshold attention state level.

107. The method of claim 106, wherein step (d) comprises presenting impulse/inhibition challenge tasks to the user after the user has reached a predetermined threshold attention state level for a predetermined length of time.

108. The method of any one of claims 96-107, wherein the first story line comprises a peer character presented to provide guidance and motivation to the user.

109. The method of claim 108, wherein the first story line and the second story line comprise a mentor character configured to encourage the user to engage in problem solving and be self-motivated.

110. The method of any one of claims 96-109, wherein step (f) further comprises, on the basis of the user response to the tasks presented in the skill transfer module, calculating a skill transfer score for the user, wherein achieving a transfer score above a predetermined threshold attention level permits the user to advance to the next level of the computer-based virtual learning curriculum.

111. The method of any one of claims 96-110, wherein the skill training module is configured to train focused and sustained attention maintenance and the skill transfer module is configured for the user to demonstrate retention of the skill trained in the training module.

112. The method of any one of claims 96-111, wherein the skill training module is configured to train behavioral inhibition and the skill transfer module is configured for the user to demonstrate retention of the skill of behavioral inhibition.

113. The method of any one of claims 96-112, wherein the skill training module is configured to train selective attention and the skill transfer module is configured for the user to demonstrate retention of the skill of selective attention.

114. The method of any one of claims 96-113, wherein the skill training module is configured to train alternating attention and the skill transfer module is configured for the user to demonstrate retention of the skill of alternating attention.

115. The method of any one of claims 96-114, wherein the skill training module is configured to train novelty inhibition and the skill transfer module is configured for the user to demonstrate retention of the skill of novelty inhibition.

116. The method of any one of claims 96-115, wherein the skill training module is configured to train delay of gratification and the skill transfer module is configured for the user to demonstrate retention of the skill of delay of gratification.

117. The method of any one of claims 96-116, wherein the skill training module is configured to train self-regulation and the skill transfer module is configured for the user to demonstrate retention of the skill of self-regulation.

118. The method of any of claims 96-117, wherein the modules are comprised of one or more levels, each level optionally being comprised of one more missions.

119. The method of claim 118, wherein the levels are designed to teach the user targeted cognitive skills, the targeted cognitive skills comprising focused attention, sustained attention, cognitive inhibition, behavioral inhibition, selective attention, alternating attention, divided attention, interference control, novelty inhibition, delay of gratification, inner voice, motivational inhibition, or self-regulation.

120. The method of any one of claims 96-119, wherein steps (a) to (f) are repeated for at least one targeted cognitive skill.

121. The method of any one of claims 96-119, wherein steps (a) to (f) are repeated for two or more targeted cognitive skills.

122. The method of any one of claims 96-121, wherein the user has ADHD and the method is performed by the user in an amount or frequency sufficient to reduce at least one of inattention, impulsivity, or hyperactivity in the user as measured by the ADHD rating scale.

123. The method of claim 122, wherein the method is performed in 3 to 7 sessions per week for 10 to 60 minutes per session, over a period of 3 to 8 weeks to treat at least one of inattention, impulsivity, or hyperactivity in the user.

124. The method of any of claims 96-123, wherein the skill training module comprises (i) providing a score of a user's performance, and (ii) based on the score, selecting a difficulty level for the skill training module.

125. The method of any of claims 96-124, wherein the user's skills performance score is quantified using a combination of (i) the user accuracy in correctly distinguishing between various stimuli, and (ii) the ability of the user to inhibit impulsive responses.

126. The method of any of claim 1-67 or 72-125, wherein the user undergoes training in two or more sessions over a number of days and for each of the two or more sessions a global attention score is calculated.

127. The method of claim 126, wherein the user undergoes training in two or more sessions over a number of days and for each of the two or more sessions a global attention score and a global composite score are calculated.

128. The method of claim 127, further comprising, for each of the two or more sessions, generating a mission performance report including the global attention score and the global composite score.

129. The method of claim 128, further comprising generating a summary progress report depicting the change in the global attention score and the global composite score achieved by the user across the two or more training sessions.

130. A game based system for treating an inattention disorder in a user in need thereof, the system comprising a processor equipped with an algorithm for presenting a computer-based virtual learning curriculum according to the method of any one of claims 71-129.

131. The game based system of claim 130, further comprising an EEG headset for collecting and communicating EEG data from the user to computing and video display device.

132. A game based system for treating an inattention, impulsivity and hyperactivity disorder in a user in need thereof, the system comprising a reporting system illustrating the user's progress in developing the underlying cognitive skills of attention and impulsivity in a virtual learning curriculum, adherence to the learning curriculum, and targeted cognitive skill levels successfully demonstrated at any point during the learning curriculum, according to the method of any one of claim 1-67 or 71-131.

133. The game based system of claim 132, wherein the reporting system is a medical or clinical professional reporting system.

134. The game based system of claim 133, wherein the reporting system is non-clinical reporting system.

135. A game based system for treating an inattention, impulsivity and hyperactivity disorder in a user in need thereof, the system comprising a parent, teacher, user, or other interested party reporting system illustrating the user training program adherence and cognitive skill levels retained at any point during the training program, the system comprising a processor equipped with an algorithm for presenting a computer-based virtual learning curriculum according to the method of any one of claim 1-67 or 71-131.

136. The game based system of claim 135, further comprising an EEG headset for collecting and communicating EEG data from the user to a computing and video display device.

137. The game based system of any one of claim 68-70 or 130-136, further comprising a computer for recording and reporting the number of training sessions undertaken by a user and the length of the training sessions.

138. The game based system of any one of claim 68-70 or 130-137, further comprising a session planner for scheduling training sessions by a user and reminding the user or third party of scheduled sessions.
